SEQUENCE LISTING (143 sequences)

SEQ ID NO: 1 - DNA, 38 bp, synthetic construct
  source 1..38: mol_type = other DNA
  misc_feature 24..26: note = DNA
  misc_feature order(1..23,27..38): note = RNA
  modified_base 25: mod_base = OTHER; note = DNA nucleotide carrying a 6-amino-5-nitro-3-yl-2(1H)-pyridone nucleobase
  modified_base order(1..23,27..38): mod_base = OTHER; note = 2-Prime-O-Methyl modified nucleoside
  modified_base order(1..7,15..20,31..38): mod_base = OTHER; note = Phosphorothioate linkage
caaggtgatgacgatcactgtcgcnatgacaacaccgc

SEQ ID NO: 2 - DNA, 18 bp, synthetic construct
  source 1..18: mol_type = other DNA
  modified_base order(5,10..12): mod_base = OTHER; note = Locked Nucleic Acid
tgttgtcatagcgacagt

SEQ ID NO: 3 - DNA, 18 bp, synthetic construct
  source 1..18: mol_type = other DNA
  modified_base 10..11: mod_base = OTHER; note = Locked Nucleic Acid
tgttgtcatggcgacagt

SEQ ID NO: 4 - DNA, 20 bp, synthetic construct
  source 1..20: mol_type = other DNA
aatggcctccttcagttggg

SEQ ID NO: 5 - DNA, 17 bp, synthetic construct
  source 1..17: mol_type = other DNA
cattggactcatggtgg

SEQ ID NO: 6 - DNA, 16 bp, synthetic construct
  source 1..16: mol_type = other DNA
cagcatcaccaaggtg

SEQ ID NO: 7 - DNA, 25 bp, synthetic construct
  source 1..25: mol_type = other DNA
  misc_feature 12..13: note = DNA
  misc_feature order(1..11,14..25): note = RNA
  modified_base 16: mod_base = OTHER; note = 2-Prime-O-Methoxyethyl modified nucleoside
  modified_base 11: mod_base = OTHER; note = 2-Prime-O-Methoxyethyl modified 5-methyl Cytidine
  modified_base 12: mod_base = OTHER; note = DNA nucleotide carrying a 6-amino-5-nitro-3-yl-2(1H)-pyridone nucleobase
  modified_base order(6,8,10,15,17,19,21): mod_base = OTHER; note = 2-Prime-Fluoro modified nucleoside
  modified_base order(1..5,7,9,14,18,20,22..25): mod_base = OTHER; note = 2-Prime-O-Methyl modified nucleoside
  modified_base order(1^2,24^25): mod_base = OTHER; note = (1,3-dimethylimidazolidin-2-ylidene) Phosphoramidate linkage
  modified_base order(2..13,14..24): mod_base = OTHER; note = Phosphorothioate linkage
  modified_base 13^14: mod_base = OTHER; note = Methylphosphonate linkage
atcacggttgcnatgacaacgccgc

SEQ ID NO: 8 - DNA, 21 bp, synthetic construct
  source 1..21: mol_type = other DNA
caacatcaccagggtgatgac

SEQ ID NO: 9 - DNA, 20 bp, synthetic construct
  source 1..20: mol_type = other DNA
catcatcggactcatggtgg

SEQ ID NO: 10 - DNA, 18 bp, synthetic construct
  source 1..18: mol_type = other DNA
  modified_base order(5,10..12): mod_base = OTHER; note = Locked Nucleic Acid
cgttgtcatagcaaccgt

SEQ ID NO: 11 - DNA, 17 bp, synthetic construct
  source 1..17: mol_type = other DNA
  modified_base 10..12: mod_base = OTHER; note = Locked Nucleic Acid
cgttgtcatggcaaccg

SEQ ID NO: 12 - DNA, 20 bp, synthetic construct
  source 1..20: mol_type = other DNA
  misc_feature 12: note = RNA
gcggatggatgtttgtgaga

SEQ ID NO: 13 - DNA, 17 bp, synthetic construct
  source 1..17: mol_type = other DNA
gccacactcttagagcc

SEQ ID NO: 14 - DNA, 15 bp, synthetic construct
  source 1..15: mol_type = other DNA
  modified_base order(7,8,11,12): mod_base = OTHER; note = Locked Nucleic Acid
cacaccgtcgccaaa

SEQ ID NO: 15 - DNA, 32 bp, synthetic construct
  source 1..32: mol_type = other DNA
  misc_feature 19..20: note = DNA
  misc_feature order(1..18,21..32): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..29): mod_base = OTHER; note = 2-Prime-Fluoro modified nucleoside
  modified_base order(6..11,21,30..32): mod_base = OTHER; note = 2-Prime-O-Methyl modified nucleoside
  modified_base order(1^2,31^32): mod_base = OTHER; note = (1,3-dimethylimidazolidin-2-ylidene) Phosphoramidate linkage
  modified_base order(6..18,19^20,22..31): mod_base = OTHER; note = Phosphorothioate linkage
  modified_base 20^21: mod_base = OTHER; note = Methylphosphonate linkage
  modified_base 20: mod_base = m5u; note = 5-methyluridine
cggtcaaacctctgaggantgggttcattttt

SEQ ID NO: 16 - DNA, 32 bp, synthetic construct; identical to SEQ ID NO: 15
  source 1..32: mol_type = other DNA
  misc_feature 19..20: note = DNA
  misc_feature order(1..18,21..32): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(13,15,17,22,24,26,28): mod_base = OTHER; note = 2-Prime-Fluoro modified nucleoside
  modified_base order(6..12,14,16,21,25,27,29..32): mod_base = OTHER; note = 2-Prime-O-Methyl modified nucleoside
  modified_base order(1^2,31^32): mod_base = OTHER; note = (1,3-dimethylimidazolidin-2-ylidene) Phosphoramidate linkage
  modified_base order(6..18,19^20,22..31): mod_base = OTHER; note = Phosphorothioate linkage
  modified_base 20^21: mod_base = OTHER; note = Methylphosphonate linkage
  modified_base 20: mod_base = m5u; note = 5-methyluridine

SEQ ID NO: 17 - DNA, 32 bp, synthetic construct; identical to SEQ ID NO: 15
  source 1..32: mol_type = other DNA
  misc_feature 19..20: note = DNA
  misc_feature order(1..18,21..32): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..29): mod_base = OTHER; note = 2-Prime-Fluoro modified nucleoside
  modified_base order(6..11,21,30..32): mod_base = OTHER; note = 2-Prime-O-Methyl modified nucleoside
  modified_base order(1^2,31^32): mod_base = OTHER; note = (1,3-dimethylimidazolidin-2-ylidene) Phosphoramidate linkage
  modified_base order(6..20,21..31): mod_base = OTHER; note = Phosphorothioate linkage
  modified_base 20^21: mod_base = OTHER; note = Methylphosphonate linkage
  modified_base 20: mod_base = m5u; note = 5-methyluridine

SEQ ID NO: 18 - DNA, 32 bp, synthetic construct; identical to SEQ ID NO: 15
  source 1..32: mol_type = other DNA
  misc_feature 19..20: note = DNA
  misc_feature order(1..18,21..32): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(13,15,17,22,24,26,28): mod_base = OTHER; note = 2-Prime-Fluoro modified nucleoside
  modified_base order(6..12,14,16,21,25,27,29..32): mod_base = OTHER; note = 2-Prime-O-Methyl modified nucleoside
  modified_base order(1^2,31^32): mod_base = OTHER; note = (1,3-dimethylimidazolidin-2-ylidene) Phosphoramidate linkage
  modified_base order(6..20,21..31): mod_base = OTHER; note = Phosphorothioate linkage
  modified_base 20^21: mod_base = OTHER; note = Methylphosphonate linkage
  modified_base 20: mod_base = m5u; note = 5-methyluridine

SEQ ID NO: 19 - DNA, 30 bp, synthetic construct
  source 1..30: mol_type = other DNA
  misc_feature 17..18: note = DNA
  misc_feature order(1..16,19..30): note = RNA
  modified_base order(1,4..5,16,21): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 2: mod_base = OTHER; note = 2-Prime-O-Methoxyethyl modified 5-methyl Uridine
  modified_base 17: mod_base = OTHER; note = DNA nucleotide carrying a 6-amino-5-nitro-3-yl-2(1H)-pyridone nucleobase
  modified_base order(10..15,20,22..27): mod_base = OTHER; note = 2-Prime-Fluoro modified nucleoside
  modified_base order(6..9,19,28..30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..16,17^18,20..29): mod_base = OTHER; note = Phosphorothioate linkage
  modified_base 18^19: mod_base = OTHER; note = Methylphosphonate linkage
  modified_base 18: mod_base = m5u; note = 5-methyluridine
gtcaaacctctgaggantgggttcattttt

SEQ ID NO: 20 - DNA, 30 bp, synthetic construct; identical to SEQ ID NO: 19
  source 1..30: mol_type = other DNA
  misc_feature 17..18: note = DNA
  misc_feature order(1..16,19..30): note = RNA
  modified_base order(1,4..5,16,21): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 2: mod_base = OTHER; note = 2-Prime-O-Methoxyethyl modified 5-methyl Uridine
  modified_base 17: mod_base = OTHER; note = DNA nucleotide carrying a 6-amino-5-nitro-3-yl-2(1H)-pyridone nucleobase
  modified_base order(11,13,15,20,22,24,26): mod_base = OTHER; note = 2-Prime-Fluoro modified nucleoside
  modified_base order(6..10,12,14,19,23,25,27..30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..16,17^18,20..29): mod_base = OTHER; note = Phosphorothioate linkage
  modified_base 18^19: mod_base = OTHER; note = Methylphosphonate linkage
  modified_base 18: mod_base = m5u; note = 5-methyluridine

SEQ ID NO: 21 - DNA, 30 bp, synthetic construct; identical to SEQ ID NO: 19
  source 1..30: mol_type = other DNA
  misc_feature 17..18: note = DNA
  misc_feature order(1..16,19..30): note = RNA
  modified_base order(1,4..5,16,21): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 2: mod_base = OTHER; note = 2-Prime-O-Methoxyethyl modified 5-methyl Uridine
  modified_base 17: mod_base = OTHER; note = DNA nucleotide carrying a 6-amino-5-nitro-3-yl-2(1H)-pyridone nucleobase
  modified_base order(10..15,20,22..27): mod_base = OTHER; note = 2-Prime-Fluoro modified nucleoside
  modified_base order(6..9,19,28..30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..18,19..29): mod_base = OTHER; note = Phosphorothioate linkage
  modified_base 18^19: mod_base = OTHER; note = Methylphosphonate linkage
  modified_base 18: mod_base = m5u; note = 5-methyluridine

SEQ ID NO: 22 - DNA, 30 bp, synthetic construct; identical to SEQ ID NO: 19
  source 1..30: mol_type = other DNA
  misc_feature 17..18: note = DNA
  misc_feature order(1..16,19..30): note = RNA
  modified_base order(1,4..5,16,21): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 2: mod_base = OTHER; note = 2-Prime-O-Methoxyethyl modified 5-methyl Uridine
  modified_base 17: mod_base = OTHER; note = DNA nucleotide carrying a 6-amino-5-nitro-3-yl-2(1H)-pyridone nucleobase
  modified_base order(11,13,15,20,22,24,26): mod_base = OTHER; note = 2-Prime-Fluoro modified nucleoside
  modified_base order(6..10,12,14,19,23,25,27..30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..18,19..29): mod_base = OTHER; note = Phosphorothioate linkage
  modified_base 18^19: mod_base = OTHER; note = Methylphosphonate linkage
  modified_base 18: mod_base = m5u; note = 5-methyluridine

SEQ ID NO: 23 - DNA, 28 bp, synthetic construct
  source 1..28: mol_type = other DNA
  misc_feature 15..16: note = DNA
  misc_feature order(1..14,17..28): note = RNA
  modified_base order(2..4,14,19): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 15: mod_base = OTHER; note = DNA nucleotide carrying a 6-amino-5-nitro-3-yl-2(1H)-pyridone nucleobase
  modified_base order(8..13,18,20..25): mod_base = OTHER; note = 2-Prime-Fluoro modified nucleoside
  modified_base order(6..7,17,26..28): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..14,15^16,18..27): mod_base = OTHER; note = Phosphorothioate linkage
  modified_base 16^17: mod_base = OTHER; note = Methylphosphonate linkage
  modified_base 16: mod_base = m5u; note = 5-methyluridine
caaacctctgaggantgggttcattttt

SEQ ID NO: 24 - DNA, 28 bp, synthetic construct
  source 1..28: mol_type = other DNA
  misc_feature 15..16: note = DNA
  misc_feature order(1..14,17..28): note = RNA
  modified_base order(2..4,14,19): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 15: mod_base = OTHER; note = DNA nucleotide carrying a 6-amino-5-nitro-3-yl-2(1H)-pyridone nucleobase
  modified_base order(9,11,13,18,20,22,24): mod_base = OTHER; note = 2-Prime-Fluoro modified nucleoside
  modified_base order(6..8,10,12,17,21,23,25..28): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..14,15^16,18..27): mod_base = OTHER; note = Phosphorothioate linkage
  modified_base 16^17: mod_base = OTHER; note = Methylphosphonate linkage
  modified_base 16: mod_base = m5u; note = 5-methyluridine
caaacctctgaggantgggttcattttt

SEQ ID NO: 25 - DNA, 28 bp, synthetic construct
  source 1..28: mol_type = other DNA
  misc_feature 15..16: note = DNA
  misc_feature order(1..14,17..28): note = RNA
  modified_base order(2..4,14,19): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 15: mod_base = OTHER; note = DNA nucleotide carrying a 6-amino-5-nitro-3-yl-2(1H)-pyridone nucleobase
  modified_base order(8..13,18,20..25): mod_base = OTHER; note = 2-Prime-Fluoro modified nucleoside
  modified_base order(6..7,17,26..28): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..16,17..27): mod_base = OTHER; note = Phosphorothioate linkage
  modified_base 16^17: mod_base = OTHER; note = Methylphosphonate linkage
  modified_base 16: mod_base = m5u; note = 5-methyluridine
caaacctctgaggantgggttcattttt

SEQ ID NO: 26 - DNA, 28 bp, synthetic construct
  source 1..28: mol_type = other DNA
  misc_feature 15..16: note = DNA
  misc_feature order(1..14,17..28): note = RNA
  modified_base order(2..4,14,19): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 15: mod_base = OTHER; note = DNA nucleotide carrying a 6-amino-5-nitro-3-yl-2(1H)-pyridone nucleobase
  modified_base order(9,11,13,18,20,22,24): mod_base = OTHER; note = 2-Prime-Fluoro modified nucleoside
  modified_base order(6..8,10,12,17,21,23,25..28): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..16,17..27): mod_base = OTHER; note = Phosphorothioate linkage
  modified_base 16^17: mod_base = OTHER; note = Methylphosphonate linkage
  modified_base 16: mod_base = m5u; note = 5-methyluridine
caaacctctgaggantgggttcattttt

SEQ ID NO: 27 - DNA, 26 bp, synthetic construct
  source 1..26: mol_type = other DNA
  misc_feature 13..14: note = DNA
  misc_feature order(1..12,15..26): note = RNA
  modified_base order(1..2,12,17): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 13: mod_base = OTHER; note = DNA nucleotide carrying a 6-amino-5-nitro-3-yl-2(1H)-pyridone nucleobase
  modified_base order(6..11,16,18..23): mod_base = OTHER; note = 2-Prime-Fluoro modified nucleoside
  modified_base order(4..5,15,24..26): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(4..12,13^14,16..25): mod_base = OTHER; note = Phosphorothioate linkage
  modified_base 14^15: mod_base = OTHER; note = Methylphosphonate linkage
  modified_base 14: mod_base = m5u; note = 5-methyluridine
aacctctgaggantgggttcattttt

SEQ ID NO: 28 - DNA, 26 bp, synthetic construct
  source 1..26: mol_type = other DNA
  misc_feature 13..14: note = DNA
  misc_feature order(1..12,15..26): note = RNA
  modified_base order(1..2,12,17): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 13: mod_base = OTHER; note = DNA nucleotide carrying a 6-amino-5-nitro-3-yl-2(1H)-pyridone nucleobase
  modified_base order(7,9,11,16,18,20,22): mod_base = OTHER; note = 2-Prime-Fluoro modified nucleoside
  modified_base order(4..6,8,10,15,19,21,23..26): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(4..12,13^14,16..25): mod_base = OTHER; note = Phosphorothioate linkage
  modified_base 14^15: mod_base = OTHER; note = Methylphosphonate linkage
  modified_base 14: mod_base = m5u; note = 5-methyluridine
aacctctgaggantgggttcattttt

SEQ ID NO: 29 - DNA, 26 bp, synthetic construct
  source 1..26: mol_type = other DNA
  misc_feature 13..14: note = DNA
  misc_feature order(1..12,15..26): note = RNA
  modified_base order(1..2,12,17): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 13: mod_base = OTHER; note = DNA nucleotide carrying a 6-amino-5-nitro-3-yl-2(1H)-pyridone nucleobase
  modified_base order(6..11,16,18..23): mod_base = OTHER; note = 2-Prime-Fluoro modified nucleoside
  modified_base order(4..5,15,24..26): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(4..14,15..25): mod_base = OTHER; note = Phosphorothioate linkage
  modified_base 14^15: mod_base = OTHER; note = Methylphosphonate linkage
  modified_base 14: mod_base = m5u; note = 5-methyluridine
aacctctgaggantgggttcattttt

SEQ ID NO: 30 - DNA, 26 bp, synthetic construct
  source 1..26: mol_type = other DNA
  misc_feature 13..14: note = DNA
  misc_feature order(1..12,15..26): note = RNA
  modified_base order(1..2,12,17): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 13: mod_base = OTHER; note = DNA nucleotide carrying a 6-amino-5-nitro-3-yl-2(1H)-pyridone nucleobase
  modified_base order(7,9,11,16,18,20,22): mod_base = OTHER; note = 2-Prime-Fluoro modified nucleoside
  modified_base order(4..6,8,10,15,19,21,23..26): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(4..14,15..25): mod_base = OTHER; note = Phosphorothioate linkage
  modified_base 14^15: mod_base = OTHER; note = Methylphosphonate linkage
  modified_base 14: mod_base = m5u; note = 5-methyluridine
aacctctgaggantgggttcattttt

SEQ ID NO: 31 - DNA, 30 bp, synthetic construct
  source 1..30: mol_type = other DNA
  misc_feature 19..20: note = DNA
  misc_feature order(1..18,21..30): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..29): mod_base = OTHER; note = 2-Prime-Fluoro modified nucleoside
  modified_base order(6..11,21,30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..18,19^20,22..29): mod_base = OTHER; note = Phosphorothioate linkage
  modified_base 20^21: mod_base = OTHER; note = Methylphosphonate linkage
  modified_base 20: mod_base = m5u; note = 5-methyluridine
cggtcaaacctctgaggantgggttcattt

SEQ ID NO: 32 - DNA, 30 bp, synthetic construct; identical to SEQ ID NO: 31
  source 1..30: mol_type = other DNA
  misc_feature 19..20: note = DNA
  misc_feature order(1..18,21..30): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(13,15,17,22,24,26,28): mod_base = OTHER; note = 2-Prime-Fluoro modified nucleoside
  modified_base order(6..12,14,16,21,25,27,29..30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..18,19^20,22..29): mod_base = OTHER; note = Phosphorothioate linkage
  modified_base 20^21: mod_base = OTHER; note = Methylphosphonate linkage
  modified_base 20: mod_base = m5u; note = 5-methyluridine

SEQ ID NO: 33 - DNA, 30 bp, synthetic construct; identical to SEQ ID NO: 31
  source 1..30: mol_type = other DNA
  misc_feature 19..20: note = DNA
  misc_feature order(1..18,21..30): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..29): mod_base = OTHER; note = 2-Prime-Fluoro modified nucleoside
  modified_base order(6..11,21,30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..20,21..29): mod_base = OTHER; note = Phosphorothioate linkage
  modified_base 20^21: mod_base = OTHER; note = Methylphosphonate linkage
  modified_base 20: mod_base = m5u; note = 5-methyluridine

SEQ ID NO: 34 - DNA, 30 bp, synthetic construct; identical to SEQ ID NO: 31
  source 1..30: mol_type = other DNA
  misc_feature 19..20: note = DNA
  misc_feature order(1..18,21..30): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(13,15,17,22,24,26,28): mod_base = OTHER; note = 2-Prime-Fluoro modified nucleoside
  modified_base order(6..12,14,16,21,25,27,29..30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..20,21..29): mod_base = OTHER; note = Phosphorothioate linkage
  modified_base 20^21: mod_base = OTHER; note = Methylphosphonate linkage
  modified_base 20: mod_base = m5u; note = 5-methyluridine

SEQ ID NO: 35 - DNA, 28 bp, synthetic construct
  source 1..28: mol_type = other DNA
  misc_feature 19..20: note = DNA
  misc_feature order(1..18,21..28): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..28): mod_base = OTHER; note = 2-Prime-Fluoro modified nucleoside
  modified_base order(6..11,21): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..18,19^20,22..27): mod_base = OTHER; note = Phosphorothioate linkage
  modified_base 20^21: mod_base = OTHER; note = Methylphosphonate linkage
  modified_base 20: mod_base = m5u; note = 5-methyluridine
cggtcaaacctctgaggantgggttcat

SEQ ID NO: 36 - DNA, 28 bp, synthetic construct
  source 1..28: mol_type = other DNA
  misc_feature 19..20: note = DNA
  misc_feature order(1..18,21..28): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(13,15,17,22,24,26,28): mod_base = OTHER; note = 2-Prime-Fluoro modified nucleoside
  modified_base order(6..12,14,16,21,25,27): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..18,19^20,22..27): mod_base = OTHER; note = Phosphorothioate linkage
  modified_base 20^21: mod_base = OTHER; note = Methylphosphonate linkage
  modified_base 20: mod_base = m5u; note = 5-methyluridine
cggtcaaacctctgaggantgggttcat

SEQ ID NO: 37 - DNA, 28 bp, synthetic construct
  source 1..28: mol_type = other DNA
  misc_feature 19..20: note = DNA
  misc_feature order(1..18,21..28): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..28): mod_base = OTHER; note = 2-Prime-Fluoro modified nucleoside
  modified_base order(6..11,21): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..20,21..27): mod_base = OTHER; note = Phosphorothioate linkage
  modified_base 20^21: mod_base = OTHER; note = Methylphosphonate linkage
  modified_base 20: mod_base = m5u; note = 5-methyluridine
cggtcaaacctctgaggantgggttcat

SEQ ID NO: 38 - DNA, 28 bp, synthetic construct
  source 1..28: mol_type = other DNA
  misc_feature 19..20: note = DNA
  misc_feature order(1..18,21..28): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(13,15,17,22,24,26,28): mod_base = OTHER; note = 2-Prime-Fluoro modified nucleoside
  modified_base order(6..12,14,16,21,25,27): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..20,21..27): mod_base = OTHER; note = Phosphorothioate linkage
  modified_base 20^21: mod_base = OTHER; note = Methylphosphonate linkage
  modified_base 20: mod_base = m5u; note = 5-methyluridine
cggtcaaacctctgaggantgggttcat

SEQ ID NO: 39 - DNA, 20 bp, synthetic construct
  source 1..20: mol_type = other DNA
tccgccactcaagagacaag

SEQ ID NO: 40 - DNA, 20 bp, synthetic construct
  source 1..20: mol_type = other DNA
gcacctggtaggtgagtgag

SEQ ID NO: 41 - DNA, 20 bp, synthetic construct
  source 1..20: mol_type = other DNA
cccgctagcaacttgacctc

SEQ ID NO: 42 - DNA, 20 bp, synthetic construct
  source 1..20: mol_type = other DNA
ccaggtccacaggcatgtta

SEQ ID NO: 43 - DNA, 17 bp, synthetic construct
  source 1..17: mol_type = other DNA
  modified_base order(7..9,16): mod_base = OTHER; note = Locked Nucleic Acid
gaacccagtcctcagag

SEQ ID NO: 44 - DNA, 19 bp, synthetic construct
  source 1..19: mol_type = other DNA
  modified_base order(2,5,9..11,18): mod_base = OTHER; note = Locked Nucleic Acid
atgaacccaatcctcagag

SEQ ID NO: 45 - DNA, 20 bp, synthetic construct
  source 1..20: mol_type = other DNA
aggagtccccgctgcttgtg

SEQ ID NO: 46 - DNA, 28 bp, synthetic construct
  source 1..28: mol_type = other DNA
  misc_feature 15..16: note = DNA
  misc_feature order(1..14,17..28): note = RNA
  modified_base order(2..4,14,19): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 15: mod_base = OTHER; note = DNA nucleotide carrying a 6-amino-5-nitro-3-yl-2(1H)-pyridone nucleobase
  modified_base order(8..13,18,20..25): mod_base = OTHER; note = 2-Prime-Fluoro modified nucleoside
  modified_base order(6..7,17,26..28): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..14,15^16,18..27): mod_base = OTHER; note = Phosphorothioate linkage
  modified_base 16^17: mod_base = OTHER; note = Methylphosphonate linkage
  modified_base 16: mod_base = m5u; note = 5-methyluridine
caaacctctgaggantgggttcattttt

SEQ ID NO: 47 - DNA, 27 bp, synthetic construct
  source 1..27: mol_type = other DNA
  misc_feature 15..16: note = DNA
  misc_feature order(1..14,17..27): note = RNA
  modified_base order(2..4,14,19): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 15: mod_base = OTHER; note = DNA nucleotide carrying a 6-amino-5-nitro-3-yl-2(1H)-pyridone nucleobase
  modified_base order(8..13,18,20..25): mod_base = OTHER; note = 2-Prime-Fluoro modified nucleoside
  modified_base order(6..7,17,26..27): mod_base = OTHER; note = 2-Prime-O-Methyl modified nucleoside
  modified_base order(1^2,26^27): mod_base = OTHER; note = (1,3-dimethylimidazolidin-2-ylidene) Phosphoramidate linkage
  modified_base order(6..14,15^16,18..26): mod_base = OTHER; note = Phosphorothioate linkage
  modified_base 16^17: mod_base = OTHER; note = Methylphosphonate linkage
  modified_base 16: mod_base = m5u; note = 5-methyluridine
caaacctctgaggantgggttcatttt

SEQ ID NO: 48 - DNA, 26 bp, synthetic construct
  source 1..26: mol_type = other DNA
  misc_feature 15..16: note = DNA
  misc_feature order(1..14,17..26): note = RNA
  modified_base order(2..4,14,19): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 15: mod_base = OTHER; note = DNA nucleotide carrying a 6-amino-5-nitro-3-yl-2(1H)-pyridone nucleobase
  modified_base order(8..13,18,20..25): mod_base = OTHER; note = 2-Prime-Fluoro modified nucleoside
  modified_base order(6..7,17,26): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(6..14,15^16,18..25): mod_base = OTHER; note = Phosphorothioate linkage
  modified_base 16^17: mod_base = OTHER; note = Methylphosphonate linkage
  modified_base 16: mod_base = m5u; note = 5-methyluridine
caaacctctgaggantgggttcattt

SEQ ID NO: 49 - DNA, 27 bp, synthetic construct
  source 1..27: mol_type = other DNA
  misc_feature 14..15: note = DNA
  misc_feature order(1..13,16..27): note = RNA
  modified_base order(1..3,13,18): mod_base = OTHER; note = 2-Prime-O-Methoxyethyl modified nucleoside
  modified_base 4: mod_base = OTHER; note = 2-Prime-O-Methoxyethyl modified 5-methyl Cytidine
  modified_base 14: mod_base = OTHER; note = DNA nucleotide carrying a 6-amino-5-nitro-3-yl-2(1H)-pyridone nucleobase
  modified_base order(7..12,17,19..24): mod_base = OTHER; note = 2-Prime-Fluoro modified nucleoside
  modified_base order(5..6,16,25..27): mod_base = OTHER; note = 2-Prime-O-Methyl modified nucleoside
  modified_base order(1^2,26^27): mod_base = OTHER; note = (1,3-dimethylimidazolidin-2-ylidene) Phosphoramidate linkage
  modified_base order(5..13,14^15,17..26): mod_base = OTHER; note = Phosphorothioate linkage
  modified_base 15^16: mod_base = OTHER; note = Methylphosphonate linkage
  modified_base 15: mod_base = m5u; note = 5-methyluridine
aaacctctgaggantgggttcattttt

SEQ ID NO: 50 - DNA, 26 bp, synthetic construct
  source 1..26: mol_type = other DNA
  misc_feature 13..14: note = DNA
  misc_feature order(1..12,15..26): note = RNA
  modified_base order(1..2,12,17): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 13: mod_base = OTHER; note = DNA nucleotide carrying a 6-amino-5-nitro-3-yl-2(1H)-pyridone nucleobase
  modified_base order(6..11,16,18..23): mod_base = OTHER; note = 2-Prime-Fluoro modified nucleoside
  modified_base order(4..5,15,24..26): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(4..12,13^14,16..25): mod_base = OTHER; note = Phosphorothioate linkage
  modified_base 14^15: mod_base = OTHER; note = Methylphosphonate linkage
  modified_base 14: mod_base = m5u; note = 5-methyluridine
aacctctgaggantgggttcattttt

SEQ ID NO: 51 - DNA, 25 bp, synthetic construct
  source 1..25: mol_type = other DNA
  misc_feature 13..14: note = DNA
  misc_feature order(1..12,15..25): note = RNA
  modified_base order(1..2,12,17): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 13: mod_base = OTHER; note = DNA nucleotide carrying a 6-amino-5-nitro-3-yl-2(1H)-pyridone nucleobase
  modified_base order(6..11,16,18..23): mod_base = OTHER; note = 2-Prime-Fluoro modified nucleoside
  modified_base order(4..5,15,24..25): mod_base = OTHER; note = 2-Prime-O-Methyl modified nucleoside
  modified_base order(1^2,24^25): mod_base = OTHER; note = (1,3-dimethylimidazolidin-2-ylidene) Phosphoramidate linkage
  modified_base order(4..12,13^14,16..24): mod_base = OTHER; note = Phosphorothioate linkage
  modified_base 14^15: mod_base = OTHER; note = Methylphosphonate linkage
  modified_base 14: mod_base = m5u; note = 5-methyluridine
aacctctgaggantgggttcatttt

SEQ ID NO: 52 - DNA, 24 bp, synthetic construct
  source 1..24: mol_type = other DNA
  misc_feature 13..14: note = DNA
  misc_feature order(1..12,15..24): note = RNA
  modified_base order(1..2,12,17): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 13: mod_base = OTHER; note = DNA nucleotide carrying a 6-amino-5-nitro-3-yl-2(1H)-pyridone nucleobase
  modified_base order(6..11,16,18..23): mod_base = OTHER; note = 2-Prime-Fluoro modified nucleoside
  modified_base order(4..5,15,24): mod_base = OTHER; note = 2-Prime-O-Methyl modified nucleoside
  modified_base order(1^2,23^24): mod_base = OTHER; note = (1,3-dimethylimidazolidin-2-ylidene) Phosphoramidate linkage
  modified_base order(4..12,13^14,16..23): mod_base = OTHER; note = Phosphorothioate linkage
  modified_base 14^15: mod_base = OTHER; note = Methylphosphonate linkage
  modified_base 14: mod_base = m5u; note = 5-methyluridine
aacctctgaggantgggttcattt

SEQ ID NO: 53 - DNA, 25 bp, synthetic construct
  source 1..25: mol_type = other DNA
  misc_feature 12..13: note = DNA
  misc_feature order(1..11,14..25): note = RNA
  modified_base order(1,11,16): mod_base = OTHER; note = 2-Prime-O-Methoxyethyl modified nucleoside
  modified_base 2: mod_base = OTHER; note = 2-Prime-O-Methoxyethyl modified 5-methyl Cytidine
  modified_base 12: mod_base = OTHER; note = DNA nucleotide carrying a 6-amino-5-nitro-3-yl-2(1H)-pyridone nucleobase
  modified_base order(5..10,15,17..22): mod_base = OTHER; note = 2-Prime-Fluoro modified nucleoside
  modified_base order(3..4,14,23..25): mod_base = OTHER; note = 2-Prime-O-Methyl modified nucleoside
  modified_base order(1^2,24^25): mod_base = OTHER; note = (1,3-dimethylimidazolidin-2-ylidene) Phosphoramidate linkage
  modified_base order(3..11,12^13,15..24): mod_base = OTHER; note = Phosphorothioate linkage
  modified_base 13^14: mod_base = OTHER; note = Methylphosphonate linkage
  modified_base 13: mod_base = m5u; note = 5-methyluridine
acctctgaggantgggttcattttt

SEQ ID NO: 54 - DNA, 24 bp, synthetic construct
  source 1..24: mol_type = other DNA
  misc_feature 11..12: note = DNA
  misc_feature order(1..10,13..24): note = RNA
  modified_base order(10,15): mod_base = OTHER; note = 2-Prime-O-Methoxyethyl modified nucleoside
  modified_base 1: mod_base = OTHER; note = 2-Prime-O-Methoxyethyl modified 5-methyl Cytidine
  modified_base 11: mod_base = OTHER; note = DNA nucleotide carrying a 6-amino-5-nitro-3-yl-2(1H)-pyridone nucleobase
  modified_base order(4..9,14,16..21): mod_base = OTHER; note = 2-Prime-Fluoro modified nucleoside
  modified_base order(2..3,13,22..24): mod_base = OTHER; note = 2-Prime-O-Methyl modified nucleoside
  modified_base order(1^2,23^24): mod_base = OTHER; note = (1,3-dimethylimidazolidin-2-ylidene) Phosphoramidate linkage
  modified_base order(2..10,11^12,14..23): mod_base = OTHER; note = Phosphorothioate linkage
  modified_base 12^13: mod_base = OTHER; note = Methylphosphonate linkage
  modified_base 12: mod_base = m5u; note = 5-methyluridine
cctctgaggantgggttcattttt

SEQ ID NO: 55 - DNA, 30 bp, synthetic construct; identical to SEQ ID NO: 31
  source 1..30: mol_type = other DNA
  misc_feature 19..20: note = DNA
  misc_feature order(1..18,21..30): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..29): mod_base = OTHER; note = 2-Prime-Fluoro modified nucleoside
  modified_base order(6..11,21,30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..18,19^20,22..29): mod_base = OTHER; note = Phosphorothioate linkage
  modified_base 20^21: mod_base = OTHER; note = Methylphosphonate linkage
  modified_base 20: mod_base = m5u; note = 5-methyluridine

SEQ ID NO: 56 - DNA, 29 bp, synthetic construct
  source 1..29: mol_type = other DNA
  misc_feature 19..20: note = DNA
  misc_feature order(1..18,21..29): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..29): mod_base = OTHER; note = 2-Prime-Fluoro modified nucleoside
  modified_base order(6..11,21): mod_base = OTHER; note = 2-Prime-O-Methyl modified nucleoside
  modified_base order(1^2,28^29): mod_base = OTHER; note = (1,3-dimethylimidazolidin-2-ylidene) Phosphoramidate linkage
  modified_base order(6..18,19^20,22..28): mod_base = OTHER; note = Phosphorothioate linkage
  modified_base 20^21: mod_base = OTHER; note = Methylphosphonate linkage
  modified_base 20: mod_base = m5u; note = 5-methyluridine
cggtcaaacctctgaggantgggttcatt

SEQ ID NO: 57 - DNA, 29 bp, synthetic construct
  source 1..29: mol_type = other DNA
  misc_feature 18..19: note = DNA
  misc_feature order(1..17,20..29): note = RNA
  modified_base order(1..2,17,22): mod_base = OTHER; note = 2-Prime-O-Methoxyethyl modified nucleoside
  modified_base 4: mod_base = OTHER; note = 2-Prime-O-Methoxyethyl modified 5-methyl Cytidine
  modified_base 3: mod_base = OTHER; note = 2-Prime-O-Methoxyethyl modified 5-methyl Uridine
  modified_base 18: mod_base = OTHER; note = DNA nucleotide carrying a 6-amino-5-nitro-3-yl-2(1H)-pyridone nucleobase
  modified_base order(11..16,21,23..28): mod_base = OTHER; note = 2-Prime-Fluoro modified nucleoside
  modified_base order(5..10,20,29): mod_base = OTHER; note = 2-Prime-O-Methyl modified nucleoside
  modified_base order(1^2,28^29): mod_base = OTHER; note = (1,3-dimethylimidazolidin-2-ylidene) Phosphoramidate linkage
  modified_base order(5..17,18^19,21..28): mod_base = OTHER; note = Phosphorothioate linkage
  modified_base 19^20: mod_base = OTHER; note = Methylphosphonate linkage
  modified_base 19: mod_base = m5u; note = 5-methyluridine
ggtcaaacctctgaggantgggttcattt

SEQ ID NO: 58 - DNA, 28 bp, synthetic construct
  source 1..28: mol_type = other DNA
  misc_feature 17..18: note = DNA
  misc_feature order(1..16,19..28): note = RNA
  modified_base order(1,16,21): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 2: mod_base = OTHER; note = 2-Prime-O-Methoxyethyl modified 5-methyl Uridine
  modified_base 17: mod_base = OTHER; note = DNA nucleotide carrying a 6-amino-5-nitro-3-yl-2(1H)-pyridone nucleobase
  modified_base order(10..15,20,22..27): mod_base = OTHER; note = 2-Prime-Fluoro modified nucleoside
  modified_base order(4..9,19,28): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(4..16,17^18,20..27): mod_base = OTHER; note = Phosphorothioate linkage
  modified_base 18^19: mod_base = OTHER; note = Methylphosphonate linkage
  modified_base 18: mod_base = m5u; note = 5-methyluridine
gtcaaacctctgaggantgggttcattt

SEQ ID NO: 59 - DNA, 27 bp, synthetic construct
  source 1..27: mol_type = other DNA
  misc_feature 16..17: note = DNA
  misc_feature order(1..15,18..27): note = RNA
  modified_base order(15,20): mod_base = OTHER; note = 2-Prime-O-Methoxyethyl modified nucleoside
  modified_base 2: mod_base = OTHER; note = 2-Prime-O-Methoxyethyl modified 5-methyl Cytidine
  modified_base 1: mod_base = OTHER; note = 2-Prime-O-Methoxyethyl modified 5-methyl Uridine
  modified_base 16: mod_base = OTHER; note = DNA nucleotide carrying a 6-amino-5-nitro-3-yl-2(1H)-pyridone nucleobase
  modified_base order(9..14,19,21..26): mod_base = OTHER; note = 2-Prime-Fluoro modified nucleoside
  modified_base order(3..8,18,27): mod_base = OTHER; note = 2-Prime-O-Methyl modified nucleoside
  modified_base order(1^2,26^27): mod_base = OTHER; note = (1,3-dimethylimidazolidin-2-ylidene) Phosphoramidate linkage
  modified_base order(3..15,16^17,19..26): mod_base = OTHER; note = Phosphorothioate linkage
  modified_base 17^18: mod_base = OTHER; note = Methylphosphonate linkage
  modified_base 17: mod_base = m5u; note = 5-methyluridine
tcaaacctctgaggantgggttcattt

SEQ ID NO: 60 - DNA, 26 bp, synthetic construct
  source 1..26: mol_type = other DNA
  misc_feature 15..16: note = DNA
  misc_feature order(1..14,17..26): note = RNA
  modified_base order(14,19): mod_base = OTHER; note = 2-Prime-O-Methoxyethyl modified nucleoside
  modified_base 1: mod_base = OTHER; note = 2-Prime-O-Methoxyethyl modified 5-methyl Cytidine
  modified_base 15: mod_base = OTHER; note = DNA nucleotide carrying a 6-amino-5-nitro-3-yl-2(1H)-pyridone nucleobase
  modified_base order(8..13,18,20..25): mod_base = OTHER; note = 2-Prime-Fluoro modified nucleoside
  modified_base order(2..7,17,26): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(2..14,15^16,18..25): mod_base = OTHER; note = Phosphorothioate linkage
  modified_base 16^17: mod_base = OTHER; note = Methylphosphonate linkage
  modified_base 16: mod_base = m5u; note = 5-methyluridine
caaacctctgaggantgggttcattt

SEQ ID NO: 61 - DNA, 25 bp, synthetic construct
  source 1..25: mol_type = other DNA
  misc_feature 14..15: note = DNA
  misc_feature order(1..13,16..25): note = RNA
  modified_base order(13,18): mod_base = OTHER; note = 2-Prime-O-Methoxyethyl modified nucleoside
  modified_base 14: mod_base = OTHER; note = DNA nucleotide carrying a 6-amino-5-nitro-3-yl-2(1H)-pyridone nucleobase
  modified_base order(7..12,17,19..24): mod_base = OTHER; note = 2-Prime-Fluoro modified nucleoside
  modified_base order(1..6,16,25): mod_base = OTHER; note = 2-Prime-O-Methyl modified nucleoside
  modified_base order(1^2,24^25): mod_base = OTHER; note = (1,3-dimethylimidazolidin-2-ylidene) Phosphoramidate linkage
  modified_base order(2..13,14^15,17..24): mod_base = OTHER; note = Phosphorothioate linkage
  modified_base 15^16: mod_base = OTHER; note = Methylphosphonate linkage
  modified_base 15: mod_base = m5u; note = 5-methyluridine
aaacctctgaggantgggttcattt

SEQ ID NO: 62 - DNA, 28 bp, synthetic construct
  source 1..28: mol_type = other DNA
  misc_feature 19..20: note = DNA
  misc_feature order(1..18,21..28): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..28): mod_base = OTHER; note = 2-Prime-Fluoro modified nucleoside
  modified_base order(6..11,21): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..18,19^20,22..27): mod_base = OTHER; note = Phosphorothioate linkage
  modified_base 20^21: mod_base = OTHER; note = Methylphosphonate linkage
  modified_base 20: mod_base = m5u; note = 5-methyluridine
cggtcaaacctctgaggantgggttcat

SEQ ID NO: 63 - DNA, 27 bp, synthetic construct
  source 1..27: mol_type = other DNA
  misc_feature 19..20: note = DNA
  misc_feature order(1..18,21..27): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..27): mod_base = OTHER; note = 2-Prime-Fluoro modified nucleoside
  modified_base order(6..11,21): mod_base = OTHER; note = 2-Prime-O-Methyl modified nucleoside
  modified_base order(1^2,26^27): mod_base = OTHER; note = (1,3-dimethylimidazolidin-2-ylidene) Phosphoramidate linkage
  modified_base order(6..18,19^20,22..26): mod_base = OTHER; note = Phosphorothioate linkage
  modified_base 20^21: mod_base = OTHER; note = Methylphosphonate linkage
  modified_base 20: mod_base = m5u; note = 5-methyluridine
cggtcaaacctctgaggantgggttca

SEQ ID NO: 64 - DNA, 26 bp, synthetic construct
  source 1..26: mol_type = other DNA
  misc_feature 19..20: note = DNA
  misc_feature order(1..18,21..26): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..26): mod_base = OTHER; note = 2-Prime-Fluoro modified nucleoside
  modified_base order(6..11,21): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(6..18,19^20,22..25): mod_base = OTHER; note = Phosphorothioate linkage
  modified_base 20^21: mod_base = OTHER; note = Methylphosphonate linkage
  modified_base 20: mod_base = m5u; note = 5-methyluridine
cggtcaaacctctgaggantgggttc

SEQ ID NO: 65 - DNA, 25 bp, synthetic construct
  source 1..25: mol_type = other DNA
  misc_feature 19..20: note = DNA
  misc_feature order(1..18,21..25): note = RNA
  modified_base order(2..3,18,23): mod_base = OTHER; note = 2-Prime-O-Methoxyethyl modified nucleoside
  modified_base order(1,5): mod_base = OTHER; note = 2-Prime-O-Methoxyethyl modified 5-methyl Cytidine
  modified_base 4: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..25): mod_base = OTHER; note = 2-Prime-Fluoro modified nucleoside
  modified_base order(6..11,21): mod_base = OTHER; note = 2-Prime-O-Methyl modified nucleoside
  modified_base order(1^2,24^25): mod_base = OTHER; note = (1,3-dimethylimidazolidin-2-ylidene) Phosphoramidate linkage
  modified_base order(6..18,19^20,22..24): mod_base = OTHER; note = Phosphorothioate linkage
  modified_base 20^21: mod_base = OTHER; note = Methylphosphonate linkage
  modified_base 20: mod_base = m5u; note = 5-methyluridine
cggtcaaacctctgaggantgggtt

SEQ ID NO: 66 - DNA, 27 bp, synthetic construct
  source 1..27: mol_type = other DNA
  misc_feature 18..19: note = DNA
  misc_feature order(1..17,20..27): note = RNA
  modified_base order(1..2,17,22): mod_base = OTHER; note = 2-Prime-O-Methoxyethyl modified nucleoside
  modified_base 4: mod_base = OTHER; note = 2-Prime-O-Methoxyethyl modified 5-methyl Cytidine
  modified_base 3: mod_base = OTHER; note = 2-Prime-O-Methoxyethyl modified 5-methyl Uridine
  modified_base 18: mod_base = OTHER; note = DNA nucleotide carrying a 6-amino-5-nitro-3-yl-2(1H)-pyridone nucleobase
  modified_base order(11..16,21,23..27): mod_base = OTHER; note = 2-Prime-Fluoro modified nucleoside
  modified_base order(5..10,20): mod_base = OTHER; note = 2-Prime-O-Methyl modified nucleoside
  modified_base order(1^2,26^27): mod_base = OTHER; note = (1,3-dimethylimidazolidin-2-ylidene) Phosphoramidate linkage
  modified_base order(5..17,18^19,21..26): mod_base = OTHER; note = Phosphorothioate linkage
  modified_base 19^20: mod_base = OTHER; note = Methylphosphonate linkage
  modified_base 19: mod_base = m5u; note = 5-methyluridine
ggtcaaacctctgaggantgggttcat

SEQ ID NO: 67 - DNA, 26 bp, synthetic construct
  source 1..26: mol_type = other DNA
  misc_feature 17..18: note = DNA
  misc_feature order(1..16,19..26): note = RNA
  modified_base order(1,16,21): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 2: mod_base = OTHER; note = 2-Prime-O-Methoxyethyl modified 5-methyl Uridine
  modified_base 17: mod_base = OTHER; note = DNA nucleotide carrying a 6-amino-5-nitro-3-yl-2(1H)-pyridone nucleobase
  modified_base order(10..15,20,22..26): mod_base = OTHER; note = 2-Prime-Fluoro modified nucleoside
  modified_base order(4..9,19): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(4..16,17^18,20..25): mod_base = OTHER; note = Phosphorothioate linkage
  modified_base 18^19: mod_base = OTHER; note = Methylphosphonate linkage
  modified_base 18: mod_base = m5u; note = 5-methyluridine
gtcaaacctctgaggantgggttcat

SEQ ID NO: 68 - DNA, 25 bp, synthetic construct
  source 1..25: mol_type = other DNA
  misc_feature 16..17: note = DNA
  misc_feature order(1..15,18..25): note = RNA
  modified_base order(15,20): mod_base = OTHER; note = 2-Prime-O-Methoxyethyl modified nucleoside
  modified_base 2: mod_base = OTHER; note = 2-Prime-O-Methoxyethyl modified 5-methyl Cytidine
  modified_base 1: mod_base = OTHER; note = 2-Prime-O-Methoxyethyl modified 5-methyl Uridine
  modified_base 16: mod_base = OTHER; note = DNA nucleotide carrying a 6-amino-5-nitro-3-yl-2(1H)-pyridone nucleobase
  modified_base order(9..14,19,21..25): mod_base = OTHER; note = 2-Prime-Fluoro modified nucleoside
  modified_base order(3..8,18): mod_base = OTHER; note = 2-Prime-O-Methyl modified nucleoside
  modified_base order(1^2,24^25): mod_base = OTHER; note = (1,3-dimethylimidazolidin-2-ylidene) Phosphoramidate linkage
  modified_base order(3..15,16^17,19..24): mod_base = OTHER; note = Phosphorothioate linkage
  modified_base 17^18: mod_base = OTHER; note = Methylphosphonate linkage
  modified_base 17: mod_base = m5u; note = 5-methyluridine
tcaaacctctgaggantgggttcat

SEQ ID NO: 69 - DNA, 24 bp, synthetic construct
  source 1..24: mol_type = other DNA
  misc_feature 15..16: note = DNA
  misc_feature order(1..14,17..24): note = RNA
  modified_base order(14,19): mod_base = OTHER; note = 2-Prime-O-Methoxyethyl modified nucleoside
  modified_base 1: mod_base = OTHER; note = 2-Prime-O-Methoxyethyl modified 5-methyl Cytidine
  modified_base 15: mod_base = OTHER; note = DNA nucleotide carrying a 6-amino-5-nitro-3-yl-2(1H)-pyridone nucleobase
  modified_base order(8..13,18,20..24): mod_base = OTHER; note = 2-Prime-Fluoro modified nucleoside
  modified_base order(2..7,17): mod_base = OTHER; note = 2-Prime-O-Methyl modified nucleoside
  modified_base order(1^2,23^24): mod_base = OTHER; note = (1,3-dimethylimidazolidin-2-ylidene) Phosphoramidate linkage
  modified_base order(2..14,15^16,18..23): mod_base = OTHER; note = Phosphorothioate linkage
  modified_base 16^17: mod_base = OTHER; note = Methylphosphonate linkage
  modified_base 16: mod_base = m5u; note = 5-methyluridine
caaacctctgaggantgggttcat

SEQ ID NO: 70 - DNA, 23 bp, synthetic construct
  source 1..23: mol_type = other DNA
  misc_feature 14..15: note = DNA
  misc_feature order(1..13,16..23): note = RNA
  modified_base order(13,18): mod_base = OTHER; note = 2-Prime-O-Methoxyethyl modified nucleoside
  modified_base 14: mod_base = OTHER; note = DNA nucleotide carrying a 6-amino-5-nitro-3-yl-2(1H)-pyridone nucleobase
  modified_base order(7..12,17,19..23): mod_base = OTHER; note = 2-Prime-Fluoro modified nucleoside
  modified_base order(1..6,16): mod_base = OTHER; note = 2-Prime-O-Methyl modified nucleoside
  modified_base order(1^2,22^23): mod_base = OTHER; note = (1,3-dimethylimidazolidin-2-ylidene) Phosphoramidate linkage
  modified_base order(2..13,14^15,17..22): mod_base = OTHER; note = Phosphorothioate linkage
  modified_base 15^16: mod_base = OTHER; note = Methylphosphonate linkage
  modified_base 15: mod_base = m5u; note = 5-methyluridine
aaacctctgaggantgggttcat

SEQ ID NO: 71 - DNA, 32 bp, synthetic construct
  source 1..32: mol_type = other DNA
  misc_feature 19..20: note = DNA
  misc_feature order(1..18,21..32): note = RNA
  modified_base order(1..4,18,23): mod_base = OTHER; note = 2-Prime-O-Methoxyethyl modified nucleoside
  modified_base 5: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..29): mod_base = OTHER; note = 2-Prime-Fluoro modified nucleoside
  modified_base order(6..11,21,30..32): mod_base = OTHER; note = 2-Prime-O-Methyl modified nucleoside
  modified_base order(1^2,31^32): mod_base = OTHER; note = (1,3-dimethylimidazolidin-2-ylidene) Phosphoramidate linkage
  modified_base order(6..18,19^20,22..31): mod_base = OTHER; note = Phosphorothioate linkage
  modified_base 20^21: mod_base = OTHER; note = Methylphosphonate linkage
  modified_base 20: mod_base = m5u; note = 5-methyluridine
agaatgcacctctgaggantgggttcattttt

SEQ ID NO: 72 - DNA, 30 bp, synthetic construct
  source 1..30: mol_type = other DNA
  misc_feature 17..18: note = DNA
  misc_feature order(1..16,19..30): note = RNA
  modified_base order(1..2,4,16,21): mod_base = OTHER; note = 2-Prime-O-Methoxyethyl modified nucleoside
  modified_base 5: mod_base = OTHER; note = 2-Prime-O-Methoxyethyl modified 5-methyl Cytidine
  modified_base 3: mod_base = OTHER; note = 2-Prime-O-Methoxyethyl modified 5-methyl Uridine
  modified_base 17: mod_base = OTHER; note = DNA nucleotide carrying a 6-amino-5-nitro-3-yl-2(1H)-pyridone nucleobase
  modified_base order(10..15,20,22..27): mod_base = OTHER; note = 2-Prime-Fluoro modified nucleoside
  modified_base order(6..9,19,28..30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..16,17^18,20..29): mod_base = OTHER; note = Phosphorothioate linkage
  modified_base 18^19: mod_base = OTHER; note = Methylphosphonate linkage
  modified_base 18: mod_base = m5u; note = 5-methyluridine
aatgcacctctgaggantgggttcattttt

SEQ ID NO: 73 - DNA, 29 bp, synthetic construct
  source 1..29: mol_type = other DNA
  misc_feature 16..17: note = DNA
  misc_feature order(1..15,18..29): note = RNA
  modified_base order(1,3,15,20): mod_base = OTHER; note = 2-Prime-O-Methoxyethyl modified nucleoside
  modified_base 4: mod_base = OTHER; note = 2-Prime-O-Methoxyethyl modified 5-methyl Cytidine
  modified_base 2: mod_base = OTHER; note = 2-Prime-O-Methoxyethyl modified 5-methyl Uridine
  modified_base 16: mod_base = OTHER; note = DNA nucleotide carrying a 6-amino-5-nitro-3-yl-2(1H)-pyridone nucleobase
  modified_base order(9..14,19,21..26): mod_base = OTHER; note = 2-Prime-Fluoro modified nucleoside
  modified_base order(5..8,18,27..29): mod_base = OTHER; note = 2-Prime-O-Methyl modified nucleoside
  modified_base order(1^2,28^29): mod_base = OTHER; note = (1,3-dimethylimidazolidin-2-ylidene) Phosphoramidate linkage
  modified_base order(7..15,16^17,19..28): mod_base = OTHER; note = Phosphorothioate linkage
  modified_base 17^18: mod_base = OTHER; note = Methylphosphonate linkage
  modified_base 17: mod_base = m5u; note = 5-methyluridine
atgcacctctgaggantgggttcattttt

SEQ ID NO: 74 - DNA, 28 bp, synthetic construct
  source 1..28: mol_type = other DNA
  misc_feature 15..16: note = DNA
  misc_feature order(1..14,17..28): note = RNA
  modified_base order(2,4,14,19): mod_base = OTHER; note = 2-Prime-O-Methoxyethyl modified nucleoside
  modified_base order(3,5): mod_base = OTHER; note = 2-Prime-O-Methoxyethyl modified 5-methyl Cytidine
  modified_base 1: mod_base = OTHER; note = 2-Prime-O-Methoxyethyl modified 5-methyl Uridine
  modified_base 15: mod_base = OTHER; note = DNA nucleotide carrying a 6-amino-5-nitro-3-yl-2(1H)-pyridone nucleobase
  modified_base order(8..13,18,20..25): mod_base = OTHER; note = 2-Prime-Fluoro modified nucleoside
  modified_base order(6..7,17,26..28): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..14,15^16,18..27): mod_base = OTHER; note = Phosphorothioate linkage
  modified_base 16^17: mod_base = OTHER; note = Methylphosphonate linkage
  modified_base 16: mod_base = m5u; note = 5-methyluridine
tgcacctctgaggantgggttcattttt

SEQ ID NO: 75 - DNA, 27 bp, synthetic construct
  source 1..27: mol_type = other DNA
  misc_feature 14..15: note = DNA
  misc_feature order(1..13,16..27): note = RNA
  modified_base order(1,3,13,18): mod_base = OTHER; note = 2-Prime-O-Methoxyethyl modified nucleoside
  modified_base order(2,4): mod_base = OTHER; note = 2-Prime-O-Methoxyethyl modified 5-methyl Cytidine
  modified_base 14: mod_base = OTHER; note = DNA nucleotide carrying a 6-amino-5-nitro-3-yl-2(1H)-pyridone nucleobase
  modified_base order(7..12,17,19..24): mod_base = OTHER; note = 2-Prime-Fluoro modified nucleoside
  modified_base order(5..6,16,25..27): mod_base = OTHER; note = 2-Prime-O-Methyl modified nucleoside
  modified_base order(1^2,26^27): mod_base = OTHER; note = (1,3-dimethylimidazolidin-2-ylidene) Phosphoramidate linkage
  modified_base order(5..13,14^15,17..26): mod_base = OTHER; note = Phosphorothioate linkage
  modified_base 15^16: mod_base = OTHER; note = Methylphosphonate linkage
  modified_base 15: mod_base = m5u; note = 5-methyluridine
gcacctctgaggantgggttcattttt

SEQ ID NO: 76 - DNA, 26 bp, synthetic construct
  source 1..26: mol_type = other DNA
  misc_feature 13..14: note = DNA
  misc_feature order(1..12,15..26): note = RNA
  modified_base order(2,12,17): mod_base = OTHER; note = 2-Prime-O-Methoxyethyl modified nucleoside
  modified_base order(1,3): mod_base = OTHER; note = 2-Prime-O-Methoxyethyl modified 5-methyl Cytidine
  modified_base 13: mod_base = OTHER; note = DNA nucleotide carrying a 6-amino-5-nitro-3-yl-2(1H)-pyridone nucleobase
  modified_base order(6..11,16,18..23): mod_base = OTHER; note = 2-Prime-Fluoro modified nucleoside
  modified_base order(4..5,15,24..26): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(4..12,13^14,16..25): mod_base = OTHER; note = Phosphorothioate linkage
  modified_base 14^15: mod_base = OTHER; note = Methylphosphonate linkage
  modified_base 14: mod_base = m5u; note = 5-methyluridine
cacctctgaggantgggttcattttt

SEQ ID NO: 77 - DNA, 25 bp, synthetic construct
  source 1..25: mol_type = other DNA
  misc_feature 12..13: note = DNA
  misc_feature order(1..11,14..25): note = RNA
  modified_base order(1,11,16): mod_base = OTHER; note = 2-Prime-O-Methoxyethyl modified nucleoside
  modified_base 2: mod_base = OTHER; note = 2-Prime-O-Methoxyethyl modified 5-methyl Cytidine
  modified_base 12: mod_base = OTHER; note = DNA nucleotide carrying a 6-amino-5-nitro-3-yl-2(1H)-pyridone nucleobase
  modified_base order(5..10,15,17..22): mod_base = OTHER; note = 2-Prime-Fluoro modified nucleoside
  modified_base order(3..4,14,23..25): mod_base = OTHER; note = 2-Prime-O-Methyl modified nucleoside
  modified_base order(1^2,24^25): mod_base = OTHER; note = (1,3-dimethylimidazolidin-2-ylidene) Phosphoramidate linkage
  modified_base order(3..11,12^13,15..24): mod_base = OTHER; note = Phosphorothioate linkage
  modified_base 13^14: mod_base = OTHER; note = Methylphosphonate linkage
  modified_base 13: mod_base = m5u; note = 5-methyluridine
acctctgaggantgggttcattttt

SEQ ID NO: 78 - DNA, 29 bp, synthetic construct
  source 1..29: mol_type = other DNA
  misc_feature 17..18: note = DNA
  misc_feature order(1..16,19..29): note = RNA
  modified_base order(1..2,16,21): mod_base = OTHER; note = 2-Prime-O-Methoxyethyl modified nucleoside
  modified_base 3: mod_base = OTHER; note = 2-Prime-O-Methoxyethyl modified 5-methyl Uridine
  modified_base 17: mod_base = OTHER; note = DNA nucleotide carrying a 6-amino-5-nitro-3-yl-2(1H)-pyridone nucleobase
  modified_base order(10..15,20,22..27): mod_base = OTHER; note = 2-Prime-Fluoro modified nucleoside
  modified_base order(4..9,19,28..29): mod_base = OTHER; note = 2-Prime-O-Methyl modified nucleoside
  modified_base order(1^2,28^29): mod_base = OTHER; note = (1,3-dimethylimidazolidin-2-ylidene) Phosphoramidate linkage
  modified_base order(6..16,17^18,20..28): mod_base = OTHER; note = Phosphorothioate linkage
  modified_base 18^19: mod_base = OTHER; note = Methylphosphonate linkage
  modified_base 18: mod_base = m5u; note = 5-methyluridine
aatgcacctctgaggantgggttcatttt

SEQ ID NO: 79 - DNA, 30 bp, synthetic construct
  source 1..30: mol_type = other DNA
  misc_feature 19..20: note = DNA
  misc_feature order(1..18,21..30): note = RNA
  modified_base order(1..4,18,23): mod_base = OTHER; note = 2-Prime-O-Methoxyethyl modified nucleoside
  modified_base 5: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..29): mod_base = OTHER; note = 2-Prime-Fluoro modified nucleoside
  modified_base order(6..11,21,30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..18,19^20,22..29): mod_base = OTHER; note = Phosphorothioate linkage
  modified_base 20^21: mod_base = OTHER; note = Methylphosphonate linkage
  modified_base 20: mod_base = m5u; note = 5-methyluridine
agaatgcacctctgaggantgggttcattt

SEQ ID NO: 80 - DNA, 29 bp, synthetic construct
  source 1..29: mol_type = other DNA
  misc_feature 18..19: note = DNA
  misc_feature order(1..17,20..29): note = RNA
  modified_base order(1..3,17,22): mod_base = OTHER; note = 2-Prime-O-Methoxyethyl modified nucleoside
  modified_base 4: mod_base = OTHER; note = 2-Prime-O-Methoxyethyl modified 5-methyl Uridine
  modified_base 18: mod_base = OTHER; note = DNA nucleotide carrying a 6-amino-5-nitro-3-yl-2(1H)-pyridone nucleobase
  modified_base order(11..16,21,23..28): mod_base = OTHER; note = 2-Prime-Fluoro modified nucleoside
  modified_base order(5..10,20,29): mod_base = OTHER; note = 2-Prime-O-Methyl modified nucleoside
  modified_base order(1^2,28^29): mod_base = OTHER; note = (1,3-dimethylimidazolidin-2-ylidene) Phosphoramidate linkage
  modified_base order(5..17,18^19,21..28): mod_base = OTHER; note = Phosphorothioate linkage
  modified_base 19^20: mod_base = OTHER; note = Methylphosphonate linkage
  modified_base 19: mod_base = m5u; note = 5-methyluridine
gaatgcacctctgaggantgggttcattt

SEQ ID NO: 81 - DNA, 28 bp, synthetic construct
  source 1..28: mol_type = other DNA
  misc_feature 17..18: note = DNA
  misc_feature order(1..16,19..28): note = RNA
  modified_base order(1..2,16,21): mod_base = OTHER; note = 2-Prime-O-Methoxyethyl modified nucleoside
  modified_base 3: mod_base = OTHER; note = 2-Prime-O-Methoxyethyl modified 5-methyl Uridine
  modified_base 17: mod_base = OTHER; note = DNA nucleotide carrying a 6-amino-5-nitro-3-yl-2(1H)-pyridone nucleobase
  modified_base order(10..15,20,22..27): mod_base = OTHER; note = 2-Prime-Fluoro modified nucleoside
  modified_base order(4..9,19,28): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(4..16,17^18,20..27): mod_base = OTHER; note = Phosphorothioate linkage
  modified_base 18^19: mod_base = OTHER; note = Methylphosphonate linkage
  modified_base 18: mod_base = m5u; note = 5-methyluridine
aatgcacctctgaggantgggttcattt

SEQ ID NO: 82 - DNA, 27 bp, synthetic construct
  source 1..27: mol_type = other DNA
  misc_feature 16..17: note = DNA
  misc_feature order(1..15,18..27): note = RNA
  modified_base order(1,15,20): mod_base = OTHER; note = 2-Prime-O-Methoxyethyl modified nucleoside
  modified_base 2: mod_base = OTHER; note = 2-Prime-O-Methoxyethyl modified 5-methyl Uridine
  modified_base 16: mod_base = OTHER; note = DNA nucleotide carrying a 6-amino-5-nitro-3-yl-2(1H)-pyridone nucleobase
  modified_base order(9..14,19,21..26): mod_base = OTHER; note = 2-Prime-Fluoro modified nucleoside
  modified_base order(3..8,18,27): mod_base = OTHER; note = 2-Prime-O-Methyl modified nucleoside
  modified_base order(1^2,26^27): mod_base = OTHER; note = (1,3-dimethylimidazolidin-2-ylidene) Phosphoramidate linkage
  modified_base order(3..15,16^17,19..26): mod_base = OTHER; note = Phosphorothioate linkage
  modified_base 17^18: mod_base = OTHER; note = Methylphosphonate linkage
  modified_base 17: mod_base = m5u; note = 5-methyluridine
atgcacctctgaggantgggttcattt

SEQ ID NO: 83 - DNA, 26 bp, synthetic construct
  source 1..26: mol_type = other DNA
  misc_feature 15..16: note = DNA
  misc_feature order(1..14,17..26): note = RNA
  modified_base order(14,19): mod_base = OTHER; note = 2-Prime-O-Methoxyethyl modified nucleoside
  modified_base 1: mod_base = OTHER; note = 2-Prime-O-Methoxyethyl modified 5-methyl Uridine
  modified_base 15: mod_base = OTHER; note = DNA nucleotide carrying a 6-amino-5-nitro-3-yl-2(1H)-pyridone nucleobase
  modified_base order(8..13,18,20..25): mod_base = OTHER; note = 2-Prime-Fluoro modified nucleoside
  modified_base order(2..7,17,26): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(2..14,15^16,18..25): mod_base = OTHER; note = Phosphorothioate linkage
  modified_base 16^17: mod_base = OTHER; note = Methylphosphonate linkage
  modified_base 16: mod_base = m5u; note = 5-methyluridine
tgcacctctgaggantgggttcattt

SEQ ID NO: 84 - DNA, 25 bp, synthetic construct
  source 1..25: mol_type = other DNA
  misc_feature 14..15: note = DNA
  misc_feature order(1..13,16..25): note = RNA
  modified_base order(13,18): mod_base = OTHER; note = 2-Prime-O-Methoxyethyl modified nucleoside
  modified_base 14: mod_base = OTHER; note = DNA nucleotide carrying a 6-amino-5-nitro-3-yl-2(1H)-pyridone nucleobase
  modified_base order(7..12,17,19..24): mod_base = OTHER; note = 2-Prime-Fluoro modified nucleoside
  modified_base order(1..6,16,25): mod_base = OTHER; note = 2-Prime-O-Methyl modified nucleoside
  modified_base order(1^2,24^25): mod_base = OTHER; note = (1,3-dimethylimidazolidin-2-ylidene) Phosphoramidate linkage
  modified_base order(2..13,14^15,17..24): mod_base = OTHER; note = Phosphorothioate linkage
  modified_base 15^16: mod_base = OTHER; note = Methylphosphonate linkage
  modified_base 15: mod_base = m5u; note = 5-methyluridine
gcacctctgaggantgggttcattt

SEQ ID NO: 85 - DNA, 29 bp, synthetic construct
  source 1..29: mol_type = other DNA
  misc_feature 19..20: note = DNA
  misc_feature order(1..18,21..29): note = RNA
  modified_base order(1..4,18,23): mod_base = OTHER; note = 2-Prime-O-Methoxyethyl modified nucleoside
  modified_base 5: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..29): mod_base = OTHER; note = 2-Prime-Fluoro modified nucleoside
  modified_base order(6..11,21): mod_base = OTHER; note = 2-Prime-O-Methyl modified nucleoside
  modified_base order(1^2,28^29): mod_base = OTHER; note = (1,3-dimethylimidazolidin-2-ylidene) Phosphoramidate linkage
  modified_base order(6..18,19^20,22..28): mod_base = OTHER; note = Phosphorothioate linkage
  modified_base 20^21: mod_base = OTHER; note = Methylphosphonate linkage
  modified_base 20: mod_base = m5u; note = 5-methyluridine
agaatgcacctctgaggantgggttcatt

SEQ ID NO: 86 - DNA, 27 bp, synthetic construct
  source 1..27: mol_type = other DNA
  misc_feature 17..18: note = DNA
  misc_feature order(1..16,19..27): note = RNA
  modified_base order(1..2,16,21): mod_base = OTHER; note = 2-Prime-O-Methoxyethyl modified nucleoside
  modified_base 3: mod_base = OTHER; note = 2-Prime-O-Methoxyethyl modified 5-methyl Uridine
  modified_base 17: mod_base = OTHER; note = DNA nucleotide carrying a 6-amino-5-nitro-3-yl-2(1H)-pyridone nucleobase
  modified_base order(10..15,20,22..27): mod_base = OTHER; note = 2-Prime-Fluoro modified nucleoside
  modified_base order(4..9,19): mod_base = OTHER; note = 2-Prime-O-Methyl modified nucleoside
  modified_base order(1^2,26^27): mod_base = OTHER; note = (1,3-dimethylimidazolidin-2-ylidene) Phosphoramidate linkage
  modified_base order(4..16,17^18,20..26): mod_base = OTHER; note = Phosphorothioate linkage
  modified_base 18^19: mod_base = OTHER; note = Methylphosphonate linkage
  modified_base 18: mod_base = m5u; note = 5-methyluridine
aatgcacctctgaggantgggttcatt

SEQ ID NO: 87 - DNA, 28 bp, synthetic construct
  source 1..28: mol_type = other DNA
  misc_feature 19..20: note = DNA
  misc_feature order(1..18,21..28): note = RNA
  modified_base order(1..4,18,23): mod_base = OTHER; note = 2-Prime-O-Methoxyethyl modified nucleoside
  modified_base 5: mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..28): mod_base = OTHER; note = 2-Prime-Fluoro modified nucleoside
  modified_base order(6..11,21): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..18,19^20,22..27): mod_base = OTHER; note = Phosphorothioate linkage
  modified_base 20^21: mod_base = OTHER; note = Methylphosphonate linkage
  modified_base 20: mod_base = m5u; note = 5-methyluridine
agaatgcacctctgaggantgggttcat

SEQ ID NO: 88 - DNA, 27 bp, synthetic construct
  source 1..27: mol_type = other DNA
  misc_feature 18..19: note = DNA
  misc_feature order(1..17,20..27): note = RNA
  modified_base order(1..3,17,22): mod_base = OTHER; note = 2-Prime-O-Methoxyethyl modified nucleoside
  modified_base 4: mod_base = OTHER; note = 2-Prime-O-Methoxyethyl modified 5-methyl Uridine
  modified_base 18: mod_base = OTHER; note = DNA nucleotide carrying a 6-amino-5-nitro-3-yl-2(1H)-pyridone nucleobase
  modified_base order(11..16,21,23..27): mod_base = OTHER; note = 2-Prime-Fluoro modified nucleoside
  modified_base order(5..10,20): mod_base = OTHER; note = 2-Prime-O-Methyl modified nucleoside
  modified_base order(1^2,26^27): mod_base = OTHER; note = (1,3-dimethylimidazolidin-2-ylidene) Phosphoramidate linkage
  modified_base order(5..17,18^19,21..26): mod_base = OTHER; note = Phosphorothioate linkage
  modified_base 19^20: mod_base = OTHER; note = Methylphosphonate linkage
  modified_base 19: mod_base = m5u; note = 5-methyluridine
gaatgcacctctgaggantgggttcat

SEQ ID NO: 89 - DNA, 26 bp, synthetic construct
  source 1..26: mol_type = other DNA
  misc_feature 17..18: note = DNA
  misc_feature order(1..16,19..26): note = RNA
  modified_base order(1..2,16,21): mod_base = OTHER; note = 2-Prime-O-Methoxyethyl modified nucleoside
  modified_base 3: mod_base = OTHER; note = 2-Prime-O-Methoxyethyl modified 5-methyl Uridine
  modified_base 17: mod_base = OTHER; note = DNA nucleotide carrying a 6-amino-5-nitro-3-yl-2(1H)-pyridone nucleobase
  modified_base order(10..15,20,22..26): mod_base = OTHER; note = 2-Prime-Fluoro modified nucleoside
  modified_base order(4..9,19): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(4..16,17^18,20..25): mod_base = OTHER; note = Phosphorothioate linkage
  modified_base 18^19: mod_base = OTHER; note = Methylphosphonate linkage
  modified_base 18: mod_base = m5u; note = 5-methyluridine
aatgcacctctgaggantgggttcat

SEQ ID NO: 90 - DNA, 25 bp, synthetic construct
  source 1..25: mol_type = other DNA
  misc_feature 16..17: note = DNA
  misc_feature order(1..15,18..25): note = RNA
  modified_base order(1,15,20): mod_base = OTHER; note = 2-Prime-O-Methoxyethyl modified nucleoside
  modified_base 2: mod_base = OTHER; note = 2-Prime-O-Methoxyethyl modified 5-methyl Uridine
  modified_base 16: mod_base = OTHER; note = DNA nucleotide carrying a 6-amino-5-nitro-3-yl-2(1H)-pyridone nucleobase
  modified_base order(9..14,19,21..25): mod_base = OTHER; note = 2-Prime-Fluoro modified nucleoside
  modified_base order(3..8,18): mod_base = OTHER; note = 2-Prime-O-Methyl modified nucleoside
  modified_base order(1^2,24^25): mod_base = OTHER; note = (1,3-dimethylimidazolidin-2-ylidene) Phosphoramidate linkage
  modified_base order(3..15,16^17,19..24): mod_base = OTHER; note = Phosphorothioate linkage
  modified_base 17^18: mod_base = OTHER; note = Methylphosphonate linkage
  modified_base 17: mod_base = m5u; note = 5-methyluridine
atgcacctctgaggantgggttcat

SEQ ID NO: 91 - DNA, 32 bp, synthetic construct
  source 1..32: mol_type = other DNA
  misc_feature 19..20: note = DNA
  misc_feature order(1..18,21..32): note = RNA
  modified_base order(2..3,23): mod_base = OTHER; note = 2-Prime-O-Methoxyethyl modified nucleoside
  modified_base order(1,4..5,18): mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..29): mod_base = OTHER; note = 2-Prime-Fluoro modified nucleoside
  modified_base order(6..11,21,30..32): mod_base = OTHER; note = 2-Prime-O-Methyl modified nucleoside
  modified_base order(1^2,31^32): mod_base = OTHER; note = (1,3-dimethylimidazolidin-2-ylidene) Phosphoramidate linkage
  modified_base order(6..18,19^20,22..31): mod_base = OTHER; note = Phosphorothioate linkage
  modified_base 20^21: mod_base = OTHER; note = Methylphosphonate linkage
taatttggcccttgctctnatggacaaagtct

SEQ ID NO: 92 - DNA, 32 bp, synthetic construct; identical to SEQ ID NO: 91
  source 1..32: mol_type = other DNA
  misc_feature 19..20: note = DNA
  misc_feature order(1..18,21..32): note = RNA
  modified_base order(2..3,23): mod_base = OTHER; note = 2-Prime-O-Methoxyethyl modified nucleoside
  modified_base order(1,4..5,18): mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(13,15,17,22,24,26,28): mod_base = OTHER; note = 2-Prime-Fluoro modified nucleoside
  modified_base order(6..12,14,16,21,25,27,29..32): mod_base = OTHER; note = 2-Prime-O-Methyl modified nucleoside
  modified_base order(1^2,31^32): mod_base = OTHER; note = (1,3-dimethylimidazolidin-2-ylidene) Phosphoramidate linkage
  modified_base order(6..18,19^20,22..31): mod_base = OTHER; note = Phosphorothioate linkage
  modified_base 20^21: mod_base = OTHER; note = Methylphosphonate linkage

SEQ ID NO: 93 - DNA, 32 bp, synthetic construct; identical to SEQ ID NO: 91
  source 1..32: mol_type = other DNA
  misc_feature 19..20: note = DNA
  misc_feature order(1..18,21..32): note = RNA
  modified_base order(2..3,23): mod_base = OTHER; note = 2-Prime-O-Methoxyethyl modified nucleoside
  modified_base order(1,4..5,18): mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..29): mod_base = OTHER; note = 2-Prime-Fluoro modified nucleoside
  modified_base order(6..11,21,30..32): mod_base = OTHER; note = 2-Prime-O-Methyl modified nucleoside
  modified_base order(1^2,31^32): mod_base = OTHER; note = (1,3-dimethylimidazolidin-2-ylidene) Phosphoramidate linkage
  modified_base order(6..20,21..31): mod_base = OTHER; note = Phosphorothioate linkage
  modified_base 20^21: mod_base = OTHER; note = Methylphosphonate linkage

SEQ ID NO: 94 - DNA, 32 bp, synthetic construct; identical to SEQ ID NO: 91
  source 1..32: mol_type = other DNA
  misc_feature 19..20: note = DNA
  misc_feature order(1..18,21..32): note = RNA
  modified_base order(2..3,23): mod_base = OTHER; note = 2-Prime-O-Methoxyethyl modified nucleoside
  modified_base order(1,4..5,18): mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(13,15,17,22,24,26,28): mod_base = OTHER; note = 2-Prime-Fluoro modified nucleoside
  modified_base order(6..12,14,16,21,25,27,29..32): mod_base = OTHER; note = 2-Prime-O-Methyl modified nucleoside
  modified_base order(1^2,31^32): mod_base = OTHER; note = (1,3-dimethylimidazolidin-2-ylidene) Phosphoramidate linkage
  modified_base order(6..20,21..31): mod_base = OTHER; note = Phosphorothioate linkage
  modified_base 20^21: mod_base = OTHER; note = Methylphosphonate linkage

SEQ ID NO: 95 - DNA, 30 bp, synthetic construct
  source 1..30: mol_type = other DNA
  misc_feature 17..18: note = DNA
  misc_feature order(1..16,19..30): note = RNA
  modified_base order(1,5,21): mod_base = OTHER; note = 2-Prime-O-Methoxyethyl modified nucleoside
  modified_base order(2..4,16): mod_base = OTHER; note = 2-Prime-O-Methoxyethyl modified 5-methyl Uridine
  modified_base 17: mod_base = OTHER; note = DNA nucleotide carrying a 6-amino-5-nitro-3-yl-2(1H)-pyridone nucleobase
  modified_base order(10..15,20,22..27): mod_base = OTHER; note = 2-Prime-Fluoro modified nucleoside
  modified_base order(6..9,19,28..30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..16,17^18,20..29): mod_base = OTHER; note = Phosphorothioate linkage
  modified_base 18^19: mod_base = OTHER; note = Methylphosphonate linkage
atttggcccttgctctnatggacaaagtct

SEQ ID NO: 96 - DNA, 30 bp, synthetic construct; identical to SEQ ID NO: 95
  source 1..30: mol_type = other DNA
  misc_feature 17..18: note = DNA
  misc_feature order(1..16,19..30): note = RNA
  modified_base order(1,5,21): mod_base = OTHER; note = 2-Prime-O-Methoxyethyl modified nucleoside
  modified_base order(2..4,16): mod_base = OTHER; note = 2-Prime-O-Methoxyethyl modified 5-methyl Uridine
  modified_base 17: mod_base = OTHER; note = DNA nucleotide carrying a 6-amino-5-nitro-3-yl-2(1H)-pyridone nucleobase
  modified_base order(11,13,15,20,22,24,26): mod_base = OTHER; note = 2-Prime-Fluoro modified nucleoside
  modified_base order(6..10,12,14,19,23,25,27..30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..16,17^18,20..29): mod_base = OTHER; note = Phosphorothioate linkage
  modified_base 18^19: mod_base = OTHER; note = Methylphosphonate linkage

SEQ ID NO: 97 - DNA, 30 bp, synthetic construct; identical to SEQ ID NO: 95
  source 1..30: mol_type = other DNA
  misc_feature 17..18: note = DNA
  misc_feature order(1..16,19..30): note = RNA
  modified_base order(1,5,21): mod_base = OTHER; note = 2-Prime-O-Methoxyethyl modified nucleoside
  modified_base order(2..4,16): mod_base = OTHER; note = 2-Prime-O-Methoxyethyl modified 5-methyl Uridine
  modified_base 17: mod_base = OTHER; note = DNA nucleotide carrying a 6-amino-5-nitro-3-yl-2(1H)-pyridone nucleobase
  modified_base order(10..15,20,22..27): mod_base = OTHER; note = 2-Prime-Fluoro modified nucleoside
  modified_base order(6..9,19,28..30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..18,19..29): mod_base = OTHER; note = Phosphorothioate linkage
  modified_base 18^19: mod_base = OTHER; note = Methylphosphonate linkage

SEQ ID NO: 98 - DNA, 30 bp, synthetic construct; identical to SEQ ID NO: 95
  source 1..30: mol_type = other DNA
  misc_feature 17..18: note = DNA
  misc_feature order(1..16,19..30): note = RNA
  modified_base order(1,5,21): mod_base = OTHER; note = 2-Prime-O-Methoxyethyl modified nucleoside
  modified_base order(2..4,16): mod_base = OTHER; note = 2-Prime-O-Methoxyethyl modified 5-methyl Uridine
  modified_base 17: mod_base = OTHER; note = DNA nucleotide carrying a 6-amino-5-nitro-3-yl-2(1H)-pyridone nucleobase
  modified_base order(11,13,15,20,22,24,26): mod_base = OTHER; note = 2-Prime-Fluoro modified nucleoside
  modified_base order(6..10,12,14,19,23,25,27..30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..18,19..29): mod_base = OTHER; note = Phosphorothioate linkage
  modified_base 18^19: mod_base = OTHER; note = Methylphosphonate linkage

SEQ ID NO: 99 - DNA, 28 bp, synthetic construct
  source 1..28: mol_type = other DNA
  misc_feature 15..16: note = DNA
  misc_feature order(1..14,17..28): note = RNA
  modified_base order(3..4,19): mod_base = OTHER; note = 2-Prime-O-Methoxyethyl modified nucleoside
  modified_base 5: mod_base = OTHER; note = 2-Prime-O-Methoxyethyl modified 5-methyl Cytidine
  modified_base order(1..2,14): mod_base = OTHER; note = 2-Prime-O-Methoxyethyl modified 5-methyl Uridine
  modified_base 15: mod_base = OTHER; note = DNA nucleotide carrying a 6-amino-5-nitro-3-yl-2(1H)-pyridone nucleobase
  modified_base order(8..13,18,20..25): mod_base = OTHER; note = 2-Prime-Fluoro modified nucleoside
  modified_base order(6..7,17,26..28): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..14,15^16,18..27): mod_base = OTHER; note = Phosphorothioate linkage
  modified_base 16^17: mod_base = OTHER; note = Methylphosphonate linkage
ttggcccttgctctnatggacaaagtct

SEQ ID NO: 100 - DNA, 28 bp, synthetic construct
  source 1..28: mol_type = other DNA
  misc_feature 15..16: note = DNA
  misc_feature order(1..14,17..28): note = RNA
  modified_base order(3..4,19): mod_base = OTHER; note = 2-Prime-O-Methoxyethyl modified nucleoside
  modified_base 5: mod_base = OTHER; note = 2-Prime-O-Methoxyethyl modified 5-methyl Cytidine
  modified_base order(1..2,14): mod_base = OTHER; note = 2-Prime-O-Methoxyethyl modified 5-methyl Uridine
  modified_base 15: mod_base = OTHER; note = DNA nucleotide carrying a 6-amino-5-nitro-3-yl-2(1H)-pyridone nucleobase
  modified_base order(9,11,13,18,20,22,24): mod_base = OTHER; note = 2-Prime-Fluoro modified nucleoside
  modified_base order(6..8,10,12,17,21,23,25..28): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..14,15^16,18..27): mod_base = OTHER; note = Phosphorothioate linkage
  modified_base 16^17: mod_base = OTHER; note = Methylphosphonate linkage
ttggcccttgctctnatggacaaagtct

SEQ ID NO: 101 - DNA, 28 bp, synthetic construct
  source 1..28: mol_type = other DNA
  misc_feature 15..16: note = DNA
  misc_feature order(1..14,17..28): note = RNA
  modified_base order(3..4,19): mod_base = OTHER; note = 2-Prime-O-Methoxyethyl modified nucleoside
  modified_base 5: mod_base = OTHER; note = 2-Prime-O-Methoxyethyl modified 5-methyl Cytidine
  modified_base order(1..2,14): mod_base = OTHER; note = 2-Prime-O-Methoxyethyl modified 5-methyl Uridine
  modified_base 15: mod_base = OTHER; note = DNA nucleotide carrying a 6-amino-5-nitro-3-yl-2(1H)-pyridone nucleobase
  modified_base order(8..13,18,20..25): mod_base = OTHER; note = 2-Prime-Fluoro modified nucleoside
  modified_base order(6..7,17,26..28): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..16,17..27): mod_base = OTHER; note = Phosphorothioate linkage
  modified_base 16^17: mod_base = OTHER; note = Methylphosphonate linkage
ttggcccttgctctnatggacaaagtct

SEQ ID NO: 102 - DNA, 28 bp, synthetic construct
  source 1..28: mol_type = other DNA
  misc_feature 15..16: note = DNA
  misc_feature order(1..14,17..28): note = RNA
  modified_base order(3..4,19): mod_base = OTHER; note = 2-Prime-O-Methoxyethyl modified nucleoside
  modified_base 5: mod_base = OTHER; note = 2-Prime-O-Methoxyethyl modified 5-methyl Cytidine
  modified_base order(1..2,14): mod_base = OTHER; note = 2-Prime-O-Methoxyethyl modified 5-methyl Uridine
  modified_base 15: mod_base = OTHER; note = DNA nucleotide carrying a 6-amino-5-nitro-3-yl-2(1H)-pyridone nucleobase
  modified_base order(9,11,13,18,20,22,24): mod_base = OTHER; note = 2-Prime-Fluoro modified nucleoside
  modified_base order(6..8,10,12,17,21,23,25..28): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..16,17..27): mod_base = OTHER; note = Phosphorothioate linkage
  modified_base 16^17: mod_base = OTHER; note = Methylphosphonate linkage
ttggcccttgctctnatggacaaagtct

SEQ ID NO: 103 - DNA, 26 bp, synthetic construct
  source 1..26: mol_type = other DNA
  misc_feature 13..14: note = DNA
  misc_feature order(1..12,15..26): note = RNA
  modified_base order(1..2,17): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 12: mod_base = OTHER; note = 2-Prime-O-Methoxyethyl modified 5-methyl Uridine
  modified_base 13: mod_base = OTHER; note = DNA nucleotide carrying a 6-amino-5-nitro-3-yl-2(1H)-pyridone nucleobase
  modified_base order(6..11,16,18..23): mod_base = OTHER; note = 2-Prime-Fluoro modified nucleoside
  modified_base order(4..5,15,24..26): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(4..12,13^14,16..25): mod_base = OTHER; note = Phosphorothioate linkage
  modified_base 14^15: mod_base = OTHER; note = Methylphosphonate linkage
ggcccttgctctnatggacaaagtct

SEQ ID NO: 104 - DNA, 26 bp, synthetic construct
  source 1..26: mol_type = other DNA
  misc_feature 13..14: note = DNA
  misc_feature order(1..12,15..26): note = RNA
  modified_base order(1..2,17): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 12: mod_base = OTHER; note = 2-Prime-O-Methoxyethyl modified 5-methyl Uridine
  modified_base 13: mod_base = OTHER; note = DNA nucleotide carrying a 6-amino-5-nitro-3-yl-2(1H)-pyridone nucleobase
  modified_base order(7,9,11,16,18,20,22): mod_base = OTHER; note = 2-Prime-Fluoro modified nucleoside
  modified_base order(4..6,8,10,15,19,21,23..26): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(4..12,13^14,16..25): mod_base = OTHER; note = Phosphorothioate linkage
  modified_base 14^15: mod_base = OTHER; note = Methylphosphonate linkage
ggcccttgctctnatggacaaagtct

SEQ ID NO: 105 - DNA, 26 bp, synthetic construct
  source 1..26: mol_type = other DNA
  misc_feature 13..14: note = DNA
  misc_feature order(1..12,15..26): note = RNA
  modified_base order(1..2,17): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 12: mod_base = OTHER; note = 2-Prime-O-Methoxyethyl modified 5-methyl Uridine
  modified_base 13: mod_base = OTHER; note = DNA nucleotide carrying a 6-amino-5-nitro-3-yl-2(1H)-pyridone nucleobase
  modified_base order(6..11,16,18..23): mod_base = OTHER; note = 2-Prime-Fluoro modified nucleoside
  modified_base order(4..5,15,24..26): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(4..14,15..25): mod_base = OTHER; note = Phosphorothioate linkage
  modified_base 14^15: mod_base = OTHER; note = Methylphosphonate linkage
ggcccttgctctnatggacaaagtct

SEQ ID NO: 106 - DNA, 26 bp, synthetic construct
  source 1..26: mol_type = other DNA
  misc_feature 13..14: note = DNA
  misc_feature order(1..12,15..26): note = RNA
  modified_base order(1..2,17): mod_base = OTHER; note = 2-Prime-O-Methoxyethyl modified nucleoside
  modified_base 3: mod_base = OTHER; note = 2-Prime-O-Methoxyethyl modified 5-methyl Cytidine
  modified_base 12: mod_base = OTHER; note = 2-Prime-O-Methoxyethyl modified 5-methyl Uridine
  modified_base 13: mod_base = OTHER; note = DNA nucleotide carrying a 6-amino-5-nitro-3-yl-2(1H)-pyridone nucleobase
  modified_base order(7,9,11,16,18,20,22): mod_base = OTHER; note = 2-Prime-Fluoro modified nucleoside
  modified_base order(4..6,8,10,15,19,21,23..26): mod_base = OTHER; note = 2-Prime-O-Methyl modified nucleoside
  modified_base order(1^2,25^26): mod_base = OTHER; note = (1,3-dimethylimidazolidin-2-ylidene) Phosphoramidate linkage
  modified_base order(4..14,15..25): mod_base = OTHER; note = Phosphorothioate linkage
  modified_base 14^15: mod_base = OTHER; note = Methylphosphonate linkage
ggcccttgctctnatggacaaagtct

SEQ ID NO: 107 - DNA, 30 bp, synthetic construct
  source 1..30: mol_type = other DNA
  misc_feature 19..20: note = DNA
  misc_feature order(1..18,21..30): note = RNA
  modified_base order(2..3,23): mod_base = OTHER; note = 2-Prime-O-Methoxyethyl modified nucleoside
  modified_base order(1,4..5,18): mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..29): mod_base = OTHER; note = 2-Prime-Fluoro modified nucleoside
  modified_base order(6..11,21,30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..18,19^20,22..29): mod_base = OTHER; note = Phosphorothioate linkage
  modified_base 20^21: mod_base = OTHER; note = Methylphosphonate linkage
taatttggcccttgctctnatggacaaagt

SEQ ID NO: 108 - DNA, 30 bp, synthetic construct; identical to SEQ ID NO: 107
  source 1..30: mol_type = other DNA
  misc_feature 19..20: note = DNA
  misc_feature order(1..18,21..30): note = RNA
  modified_base order(2..3,23): mod_base = OTHER; note = 2-Prime-O-Methoxyethyl modified nucleoside
  modified_base order(1,4..5,18): mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(13,15,17,22,24,26,28): mod_base = OTHER; note = 2-Prime-Fluoro modified nucleoside
  modified_base order(6..12,14,16,21,25,27,29..30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..18,19^20,22..29): mod_base = OTHER; note = Phosphorothioate linkage
  modified_base 20^21: mod_base = OTHER; note = Methylphosphonate linkage

SEQ ID NO: 109 - DNA, 30 bp, synthetic construct; identical to SEQ ID NO: 107
  source 1..30: mol_type = other DNA
  misc_feature 19..20: note = DNA
  misc_feature order(1..18,21..30): note = RNA
  modified_base order(2..3,23): mod_base = OTHER; note = 2-Prime-O-Methoxyethyl modified nucleoside
  modified_base order(1,4..5,18): mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..29): mod_base = OTHER; note = 2-Prime-Fluoro modified nucleoside
  modified_base order(6..11,21,30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..20,21..29): mod_base = OTHER; note = Phosphorothioate linkage
  modified_base 20^21: mod_base = OTHER; note = Methylphosphonate linkage

SEQ ID NO: 110 - DNA, 30 bp, synthetic construct; identical to SEQ ID NO: 107
  source 1..30: mol_type = other DNA
  misc_feature 19..20: note = DNA
  misc_feature order(1..18,21..30): note = RNA
  modified_base order(2..3,23): mod_base = OTHER; note = 2-Prime-O-Methoxyethyl modified nucleoside
  modified_base order(1,4..5,18): mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(13,15,17,22,24,26,28): mod_base = OTHER; note = 2-Prime-Fluoro modified nucleoside
  modified_base order(6..12,14,16,21,25,27,29..30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(6..20,21..29): mod_base = OTHER; note = Phosphorothioate linkage
  modified_base 20^21: mod_base = OTHER; note = Methylphosphonate linkage

SEQ ID NO: 111 - DNA, 28 bp, synthetic construct
  source 1..28: mol_type = other DNA
  misc_feature 19..20: note = DNA
  misc_feature order(1..18,21..28): note = RNA
  modified_base order(2..3,23): mod_base = OTHER; note = 2-Prime-O-Methoxyethyl modified nucleoside
  modified_base order(1,4..5,18): mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..28): mod_base = OTHER; note = 2-Prime-Fluoro modified nucleoside
  modified_base order(6..11,21): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..18,19^20,22..27): mod_base = OTHER; note = Phosphorothioate linkage
  modified_base 20^21: mod_base = OTHER; note = Methylphosphonate linkage
taatttggcccttgctctnatggacaaa

SEQ ID NO: 112 - DNA, 28 bp, synthetic construct
  source 1..28: mol_type = other DNA
  misc_feature 19..20: note = DNA
  misc_feature order(1..18,21..28): note = RNA
  modified_base order(2..3,23): mod_base = OTHER; note = 2-Prime-O-Methoxyethyl modified nucleoside
  modified_base order(1,4..5,18): mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(13,15,17,22,24,26,28): mod_base = OTHER; note = 2-Prime-Fluoro modified nucleoside
  modified_base order(6..12,14,16,21,25,27): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..18,19^20,22..27): mod_base = OTHER; note = Phosphorothioate linkage
  modified_base 20^21: mod_base = OTHER; note = Methylphosphonate linkage
taatttggcccttgctctnatggacaaa

SEQ ID NO: 113 - DNA, 28 bp, synthetic construct
  source 1..28: mol_type = other DNA
  misc_feature 19..20: note = DNA
  misc_feature order(1..18,21..28): note = RNA
  modified_base order(2..3,23): mod_base = OTHER; note = 2-Prime-O-Methoxyethyl modified nucleoside
  modified_base order(1,4..5,18): mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(12..17,22,24..28): mod_base = OTHER; note = 2-Prime-Fluoro modified nucleoside
  modified_base order(6..11,21): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..20,21..27): mod_base = OTHER; note = Phosphorothioate linkage
  modified_base 20^21: mod_base = OTHER; note = Methylphosphonate linkage
taatttggcccttgctctnatggacaaa

SEQ ID NO: 114 - DNA, 28 bp, synthetic construct
  source 1..28: mol_type = other DNA
  misc_feature 19..20: note = DNA
  misc_feature order(1..18,21..28): note = RNA
  modified_base order(2..3,23): mod_base = OTHER; note = 2-Prime-O-Methoxyethyl modified nucleoside
  modified_base order(1,4..5,18): mod_base = OTHER; note = 2-Prime-O-Methoxyethyl modified 5-methyl Uridine
  modified_base 19: mod_base = OTHER; note = DNA nucleotide carrying a 6-amino-5-nitro-3-yl-2(1H)-pyridone nucleobase
  modified_base order(13,15,17,22,24,26,28): mod_base = OTHER; note = 2-Prime-Fluoro modified nucleoside
  modified_base order(6..12,14,16,21,25,27): mod_base = OTHER; note = 2-Prime-O-Methyl modified nucleoside
  modified_base order(1^2,27^28): mod_base = OTHER; note = (1,3-dimethylimidazolidin-2-ylidene) Phosphoramidate linkage
  modified_base order(6..20,21..27): mod_base = OTHER; note = Phosphorothioate linkage
  modified_base 20^21: mod_base = OTHER; note = Methylphosphonate linkage
taatttggcccttgctctnatggacaaa

SEQ ID NO: 115 - DNA, 30 bp, synthetic construct
  source 1..30: mol_type = other DNA
  misc_feature 24..25: note = DNA
  misc_feature order(1..23,26..30): note = RNA
  modified_base 28: mod_base = OTHER; note = 2-Prime-O-Methoxyethyl modified nucleoside
  modified_base 23: mod_base = OTHER; note = 2-Prime-O-Methoxyethyl modified 5-methyl Uridine
  modified_base 24: mod_base = OTHER; note = DNA nucleotide carrying a 6-amino-5-nitro-3-yl-2(1H)-pyridone nucleobase
  modified_base order(5..6,9..10,13..14,17..22,27): mod_base = OTHER; note = 2-Prime-Fluoro modified nucleoside
  modified_base order(1..4,7..8,11..12,15..16,26,29..30): mod_base = OTHER; note = 2-Prime-O-Methyl modified nucleoside
  modified_base order(1^2,29^30): mod_base = OTHER; note = (1,3-dimethylimidazolidin-2-ylidene) Phosphoramidate linkage
  modified_base order(2..23,24^25,27..29): mod_base = OTHER; note = Phosphorothioate linkage
  modified_base 25^26: mod_base = OTHER; note = Methylphosphonate linkage
gtcattaatttggcccttgctctnatggac

SEQ ID NO: 116 - DNA, 30 bp, synthetic construct; identical to SEQ ID NO: 115
  source 1..30: mol_type = other DNA
  misc_feature 24..25: note = DNA
  misc_feature order(1..23,26..30): note = RNA
  modified_base 28: mod_base = OTHER; note = 2-Prime-O-Methoxyethyl modified nucleoside
  modified_base 23: mod_base = OTHER; note = 2-Prime-O-Methoxyethyl modified 5-methyl Uridine
  modified_base 24: mod_base = OTHER; note = DNA nucleotide carrying a 6-amino-5-nitro-3-yl-2(1H)-pyridone nucleobase
  modified_base order(4..5,8..9,11..15,18..19,22,27): mod_base = OTHER; note = 2-Prime-Fluoro modified nucleoside
  modified_base order(1..3,6..7,10,16..17,20..21,26,29..30): mod_base = OTHER; note = 2-Prime-O-Methyl modified nucleoside
  modified_base order(1^2,11^12,14^15,29^30): mod_base = OTHER; note = (1,3-dimethylimidazolidin-2-ylidene) Phosphoramidate linkage
  modified_base order(2..6,8..11,12^13,18..20,21..23,24^25,27..29): mod_base = OTHER; note = Phosphorothioate linkage
  modified_base 25^26: mod_base = OTHER; note = Methylphosphonate linkage

SEQ ID NO: 117 - DNA, 30 bp, synthetic construct; identical to SEQ ID NO: 115
  source 1..30: mol_type = other DNA
  misc_feature 24..25: note = DNA
  misc_feature order(1..23,26..30): note = RNA
  modified_base 28: mod_base = OTHER; note = 2-Prime-O-Methoxyethyl modified nucleoside
  modified_base 23: mod_base = OTHER; note = 2-Prime-O-Methoxyethyl modified 5-methyl Uridine
  modified_base 24: mod_base = OTHER; note = DNA nucleotide carrying a 6-amino-5-nitro-3-yl-2(1H)-pyridone nucleobase
  modified_base order(4..6,8..9,11,13..15,18..20,22,27): mod_base = OTHER; note = 2-Prime-Fluoro modified nucleoside
  modified_base order(1..3,7,10,12,16..17,21,26,29..30): mod_base = OTHER; note = 2-Prime-O-Methyl modified nucleoside
  modified_base order(1^2,11^12,14^15,29^30): mod_base = OTHER; note = (1,3-dimethylimidazolidin-2-ylidene) Phosphoramidate linkage
  modified_base order(2..6,7..9,10^11,13^14,16..21,22^23,24^25,27..29): mod_base = OTHER; note = Phosphorothioate linkage
  modified_base 25^26: mod_base = OTHER; note = Methylphosphonate linkage

SEQ ID NO: 118 - DNA, 30 bp, synthetic construct; identical to SEQ ID NO: 115
  source 1..30: mol_type = other DNA
  misc_feature 24..25: note = DNA
  misc_feature order(1..23,26..30): note = RNA
  modified_base order(7,28): mod_base = OTHER; note = 2-Prime-O-Methoxyethyl modified nucleoside
  modified_base 16: mod_base = OTHER; note = 2-Prime-O-Methoxyethyl modified 5-methyl Cytidine
  modified_base order(6,21,23): mod_base = OTHER; note = 2-Prime-O-Methoxyethyl modified 5-methyl Uridine
  modified_base 24: mod_base = OTHER; note = DNA nucleotide carrying a 6-amino-5-nitro-3-yl-2(1H)-pyridone nucleobase
  modified_base order(4..5,8..9,13..15,17,19,22,27): mod_base = OTHER; note = 2-Prime-Fluoro modified nucleoside
  modified_base order(1..3,10..12,18,20,26,29..30): mod_base = OTHER; note = 2-Prime-O-Methyl modified nucleoside
  modified_base order(1^2,11^12,14^15,19^20,29^30): mod_base = OTHER; note = (1,3-dimethylimidazolidin-2-ylidene) Phosphoramidate linkage
  modified_base order(2..6,8..10,13^14,16..18,22^23,24^25,27..29): mod_base = OTHER; note = Phosphorothioate linkage
  modified_base 25^26: mod_base = OTHER; note = Methylphosphonate linkage

SEQ ID NO: 119 - DNA, 30 bp, synthetic construct; identical to SEQ ID NO: 115
  source 1..30: mol_type = other DNA
  misc_feature 24..25: note = DNA
  misc_feature order(1..23,26..30): note = RNA
  modified_base order(7,28): mod_base = OTHER; note = 2-Prime-O-Methoxyethyl modified nucleoside
  modified_base order(16,20): mod_base = OTHER; note = 2-Prime-O-Methoxyethyl modified 5-methyl Cytidine
  modified_base order(6,10,21,23): mod_base = OTHER; note = 2-Prime-O-Methoxyethyl modified 5-methyl Uridine
  modified_base 24: mod_base = OTHER; note = DNA nucleotide carrying a 6-amino-5-nitro-3-yl-2(1H)-pyridone nucleobase
  modified_base order(4..5,8..9,11,13..15,17..19,22,27): mod_base = OTHER; note = 2-Prime-Fluoro modified nucleoside
  modified_base order(1..3,12,26,29..30): mod_base = OTHER; note = 2-Prime-O-Methyl modified nucleoside
  modified_base order(1^2,11^12,14^15,19^20,29^30): mod_base = OTHER; note = (1,3-dimethylimidazolidin-2-ylidene) Phosphoramidate linkage
  modified_base order(2..6,8..10,13^14,16..19,22^23,24^25,27..29): mod_base = OTHER; note = Phosphorothioate linkage
  modified_base 25^26: mod_base = OTHER; note = Methylphosphonate linkage

SEQ ID NO: 120 - DNA, 27 bp, synthetic construct
  source 1..27: mol_type = other DNA
  misc_feature 16..17: note = DNA
  misc_feature order(1..15,18..27): note = RNA
  modified_base order(4,20): mod_base = OTHER; note = 2-Prime-O-Methoxyethyl modified nucleoside
  modified_base order(8,12): mod_base = OTHER; note = 2-Prime-O-Methoxyethyl modified 5-methyl Cytidine
  modified_base order(1..2,9,13,15): mod_base = OTHER; note = 2-Prime-O-Methoxyethyl modified 5-methyl Uridine
  modified_base 16: mod_base = OTHER; note = DNA nucleotide carrying a 6-amino-5-nitro-3-yl-2(1H)-pyridone nucleobase
  modified_base order(5..6,14,19,21,23,25): mod_base = OTHER; note = 2-Prime-Fluoro modified nucleoside
  modified_base order(3,7,10..11,18,22,24,26..27): mod_base = OTHER; note = 2-Prime-O-Methyl modified nucleoside
  modified_base order(2^3,5^6,9..12,14..17,18..26): mod_base = OTHER; note = Phosphorothioate linkage
  modified_base order(3^4,6^7,26^27): mod_base = OTHER; note = (1,3-dimethylimidazolidin-2-ylidene) Phosphoramidate linkage
  modified_base 17^18: mod_base = OTHER; note = Methylphosphonate linkage
tttggcccttgctctnatggacaaagt

SEQ ID NO: 121 - RNA, 101 nt, Homo sapiens
  source 1..101: mol_type = mRNA
tagccaatggcctccttcagttgggacatggtcttaaagactttgtccataagacgaagggccaaattaatgacatatttcaaaaactcaacatatttgat

SEQ ID NO: 122 - DNA, 23 bp, synthetic construct
  source 1..23: mol_type = other DNA
aatagtcattccaagtatccatg

SEQ ID NO: 123 - DNA, 16 bp, synthetic construct
  source 1..16: mol_type = other DNA
gcttttgggagggtga

SEQ ID NO: 124 - DNA, 17 bp, synthetic construct
  source 1..17: mol_type = other DNA
ggctgtattcccctcca

SEQ ID NO: 125 - DNA, 20 bp, synthetic construct
  source 1..20: mol_type = other DNA
gttcaatggggtacttcagg

SEQ ID NO: 126 - DNA, 15 bp, synthetic construct
  source 1..15: mol_type = other DNA
cgagctgcttccaca

SEQ ID NO: 127 - DNA, 17 bp, synthetic construct
  source 1..17: mol_type = other DNA
gccacactcttagagcc

SEQ ID NO: 128 - DNA, 16 bp, synthetic construct
  source 1..16: mol_type = other DNA
  modified_base order(3,4,7..9,11,15): mod_base = OTHER; note = Locked Nucleic Acid
aaccacttatttcatg

SEQ ID NO: 129 - DNA, 16 bp, synthetic construct
  source 1..16: mol_type = other DNA
  modified_base order(3,7..9,11,13): mod_base = OTHER; note = Locked Nucleic Acid
aaccactcatttcatg

SEQ ID NO: 130 - DNA, 22 bp, synthetic construct
  source 1..22: mol_type = other DNA
tggtgggaatgggtcagaagga

SEQ ID NO: 131 - DNA, 20 bp, synthetic construct
  source 1..20: mol_type = other DNA
gcacctgtacctccgaggtg

SEQ ID NO: 132 - DNA, 27 bp, synthetic construct
  source 1..27: mol_type = other DNA
  misc_feature 14..15: note = DNA
  misc_feature order(1..13,16..27): note = RNA
  modified_base order(13,18): mod_base = OTHER; note = 2-Prime-O-Methoxyethyl modified 5-methyl Uridine
  modified_base 14: mod_base = OTHER; note = DNA nucleotide carrying a 6-amino-5-nitro-3-yl-2(1H)-pyridone nucleobase
  modified_base order(8,10,12,17,19,21,23): mod_base = OTHER; note = 2-Prime-Fluoro modified nucleoside
  modified_base order(1..7,9,11,16,20,22,24..27): mod_base = OTHER; note = 2-Prime-O-Methyl modified nucleoside
  modified_base order(1^2,26^27): mod_base = OTHER; note = (1,3-dimethylimidazolidin-2-ylidene) Phosphoramidate linkage
  modified_base order(2..13,14^15,17..26): mod_base = OTHER; note = Phosphorothioate linkage
  modified_base 15^16: mod_base = OTHER; note = Methylphosphonate linkage
tcctgtaaccactnatttcatggatac

SEQ ID NO: 133 - DNA, 25 bp, synthetic construct
  source 1..25: mol_type = other DNA
  misc_feature 12..13: note = DNA
  misc_feature order(1..11,14..25): note = RNA
  modified_base 16: mod_base = OTHER; note = 2-Prime-O-Methoxyethyl modified nucleoside
  modified_base 11: mod_base = OTHER; note = 2-Prime-O-Methoxyethyl modified 5-methyl Cytidine
  modified_base 12: mod_base = OTHER; note = DNA nucleotide carrying a 6-amino-5-nitro-3-yl-2(1H)-pyridone nucleobase
  modified_base order(6,8,10,15,17,19,21): mod_base = OTHER; note = 2-Prime-Fluoro modified nucleoside
  modified_base order(1..5,7,9,14,18,20,22..25): mod_base = OTHER; note = 2-Prime-O-Methyl modified nucleoside
  modified_base order(1^2,24^25): mod_base = OTHER; note = (1,3-dimethylimidazolidin-2-ylidene) Phosphoramidate linkage
  modified_base order(2..13,14..24): mod_base = OTHER; note = Phosphorothioate linkage
  modified_base 13^14: mod_base = OTHER; note = Methylphosphonate linkage
atcacggttgcnatgacaacgccgc

SEQ ID NO: 134 - DNA, 20 bp, synthetic construct
  source 1..20: mol_type = other DNA
aatggcctccttcagttggg

SEQ ID NO: 135 - DNA, 21 bp, synthetic construct
  source 1..21: mol_type = other DNA
tgatttcactggtttgcagcg

SEQ ID NO: 136 - DNA, 19 bp, synthetic construct
  source 1..19: mol_type = other DNA
  modified_base 10..12: mod_base = OTHER; note = Locked Nucleic Acid
ctttgtccatgagacgaag

SEQ ID NO: 137 - DNA, 20 bp, synthetic construct
  source 1..20: mol_type = other DNA
  modified_base 10..12: mod_base = OTHER; note = Locked Nucleic Acid
ctttgtccataagacgaagg

SEQ ID NO: 138 - DNA, 20 bp, synthetic construct
  source 1..20: mol_type = other DNA
cgattactggcaatgtcccc

SEQ ID NO: 139 - DNA, 21 bp, synthetic construct
  source 1..21: mol_type = other DNA
tctccacactcatcatgccac

SEQ ID NO: 140 - DNA, 20 bp, synthetic construct
  source 1..20: mol_type = other DNA
acttcaactgtccagagggt

SEQ ID NO: 141 - DNA, 20 bp, synthetic construct
  source 1..20: mol_type = other DNA
cctggcgtcgtgattagtga

SEQ ID NO: 142 - DNA, 20 bp, synthetic construct
  source 1..20: mol_type = other DNA
cgagcaagacgttcagtcct

SEQ ID NO: 143 - DNA, 23 bp, synthetic construct
  source 1..23: mol_type = other DNA
  modified_base 4: mod_base = OTHER; note = Locked Nucleic Acid
tgctgaggatttggaaagggtgt

(19) United States
(12) Patent Application Publication
(10) Pub. No.: US 2026/0327622 A1
(43) Pub. Date: Oct. 22, 2026

(54) DELIVERY OF OLIGONUCLEOTIDES

(71) Applicants: ProQR Therapeutics II B.V., Leiden (NL); Freie Universitat Berlin, Berlin (DE)

(72) Inventors: Peter Christian DE VISSER, Leiden (NL); Jan-Niklas DURIG, Berlin (DE); Alexander WENG, Berlin (DE); Peggy Ramona BOHLANDER, Leiden (NL)

(73) Assignees: ProQR Therapeutics II B.V., Leiden (NL); Freie Universitat Berlin, Berlin (DE)

(21) Appl. No.: 19/149,463

(22) PCT Filed: Jan. 19, 2024
(86) PCT No.: PCT/EP2024/051278

§ 371 Date: Jul. 18, 2025

(30) Foreign Application Priority Data
Jan. 20, 2023 (GB) 2300865.9

(51) Int. Cl. C12N 15/113

(52) U.S. Cl. CPC C12N 15/1137; C12N 2310/11; C12N 2310/351

(57) ABSTRACT

The invention relates to the field of medicine and to the field of RNA editing, wherein a target adenosine present in a target RNA molecule in a cell is deaminated to an inosine by an endogenous ADAR enzyme that is recruited by a double-stranded complex generated between an administered RNA editing producing antisense oligonucleotide (EON) and a region of the target RNA molecule that comprises the target adenosine. The invention relates to the improved delivery of the EON to the target cell using a triterpene glycoside purified from seeds of Agrostemma githago L.

### TECHNICAL FIELD

This invention relates to the field of medicine and particularly to the delivery of oligonucleotides for the treatment of disease. The invention involves the use of particular triterpene glycosides (also referred to as triterpene saponins), a class of secondary metabolites, mainly produced by plants, composed of one or more linear and/or branched oligo-carbohydrates and a triterpene backbone (aglycone). These triterpene glycosides improve the in vitro and in vivo delivery of therapeutic oligonucleotides and oligonucleotide complexes, preferably for RNA editing of a target nucleotide in a target transcription molecule.

### BACKGROUND

Antisense oligonucleotides (AONs, also often abbreviated to ASOs) have been developed for use in the treatment of a wide variety of diseases. AONs range from gapmers (generally for reducing expression of a target transcription product), siRNA's, small activating RNA's (saRNA's), steric blockers (compounds that can modulate splicing (such as exon-skipping inducing AONs) or translation-blocking AONs), immunostimulatory compounds, decoy oligonucleotides, miRNA inhibitors, and RNAzymes to RNA editing oligonucleotides (often abbreviated to EONs), which are, for example, applicable in the specific deamination of specified target nucleotides in a target transcript molecule, specific 2′-O-methylation, or uridine-to-pseudouridine isomerization. AONs are generally short, single-stranded molecules comprising synthetic and/or chemically modified RNA and/or DNA nucleotides or analogues thereof, which are capable of hybridizing to specific targets by H-bonding, which generally follows the Watson-Crick rules. AONs may sometimes form intramolecular structures, such as stem-loops, and may be complexed to sense strands to form (hetero) duplex oligonucleotide complexes. For some purposes, AONs may be (covalently or non-covalently) bound to functional moieties, such as GalNAc structures when delivery to hepatocytes is desired. When the delivery of an AON is performed without the help of a vector (like a viral vector) and the AON is meant to be delivered in a ‘naked’ manner, they are generally chemically modified to for instance enhance the resistance to nucleases such as RNase H. Commonly used modifications are the substitution of the 2′ position of the ribose sugar moiety with a 2′-O-methyl group (hereinafter 2′-OMe) or a 2′-O-methoxyethyl group (hereinafter 2′-MOE) and the replacement of the natural phosphodiester linkage between two nucleosides by the more resistant phosphorothioate (PS) linkage or methyl phosphonate (MP) linkage. Currently, a wide variety of chemical modifications to the nitrogenous base, the ribose sugar and/or the linkages have been proposed and are being tested in a wide variety of pre-clinical and clinical settings. However, the potential toxicity of the selected chemical modifications should always be weighed against the dosing that is required in in vivo settings to reach a relevant therapeutic effect. Hence, there is a need to increase the efficiency of delivery of AONs to their specific target cells in vivo and to increase the intracellular trafficking such that the AON when it has entered the cell can reach its target transcript molecule and bind it. In other words, it is preferred to find ways to aid in the delivery of AONs, such that dosing can potentially be lowered to still reach a significant therapeutic effect, independent of the mechanism of action of the single-stranded or double-stranded AON.

One class of compounds that has been investigated for the delivery of AONs is the class of ‘saponins’, which are organic chemicals that have a foamy quality when agitated in water and that are generally found in plants, such as the soapbark tree (Quillaja saponaria) and soybeans. Saponins are used in dietary supplements, in carbonated beverages and are commonly used as adjuvants in vaccines. The amphiphilic nature, immunologic potential, and divergent biological activities have made saponins suitable adjuvants for drug delivery, besides their reported actions as anti-inflammatory, antibacterial, antifungal, antiviral, insecticidal, anticancer and molluscicidal compounds (see for example Said Ashour A. et al. 2019 J. Nanomed. Res. 1:282-288). Shiri et al (Shiri E. et al. 2022. J Manzandaran Univ Med Sci. 32:43-54) noted that IV administered saponin as pre-treatment to rats subjected to ischemia-reperfusion could be effective in reducing damage caused by cerebral ischemia. Wang and colleagues have revealed that the use of saponins could enhance exon skipping of a chemically modified AON in vitro and in vivo (Wang M et al. 2018a. Mol Ther Nucleic Acids. 11:192-202; Wang M et al. 2018b. Drug Des Devel Ther. 12:3705-3715), showing that this class of compounds could also be applied for the delivery of oligonucleotides.

The present invention aims to provide one or more alternative and/or improved techniques, compounds and/or compositions for use in the delivery of (antisense) oligonucleotides and oligonucleotide complexes.

### SUMMARY OF THE INVENTION

According to a first aspect, the invention provides a composition comprising a triterpene glycoside and an RNA editing producing antisense oligonucleotide (EON), wherein the EON can form a double-stranded complex with a region of a target RNA molecule in a cell, wherein the region of the target RNA molecule comprises a target adenosine, wherein the nucleotide in the EON that is opposite the target adenosine is the orphan nucleotide, and wherein the double-stranded complex can bind an endogenous ADAR enzyme to deaminate the target adenosine into an inosine, thereby editing the target RNA molecule. Preferably, the triterpene glycoside is conjugated to the EON. Preferably, the triterpene glycoside is AG1856. In one embodiment, the EON is hybridized to a fully or partially complementary sense oligonucleotide to form a heteroduplex RNA editing producing oligonucleotide (HEON) complex. In one embodiment, the target RNA molecule is a pre-mRNA or mRNA target molecule.

According to a second aspect, the invention provides a composition according to the invention, for use in the treatment of a cardiovascular disease, a disease involving the liver, a disease involving the kidney, a disease involving the pancreas, or a disorder of the central nervous system. In one embodiment, the target RNA molecule is endogenously present in the cell and the target RNA molecule is transcribed from a human gene selected from the group consisting of: SERPINA1, IDUA, HFE, ABCA4, USH2A, PCSK9, B4GALT1, ALDH2, HTT, DMD, PNPLA3, APOC3, C9orf72, DMPK, RHO, MAPT, OTOF, SMN1, ASL, APP, PMP22, LRRK2, ASS1, GJB2, MECP2, and RS1.

In a third aspect, the invention provides a kit-of-parts comprising (i) a first pharmaceutical composition comprising a triterpene glycoside; and (ii) a second pharmaceutical composition comprising an EON, wherein the EON can form a double-stranded complex with a region of a target RNA molecule in a cell, wherein the region of the target RNA molecule comprises a target adenosine, and wherein the double-stranded complex can recruit an endogenous ADAR enzyme to deaminate the target adenosine into an inosine, thereby editing the target RNA molecule. Preferably, the triterpene glycoside is AG1856.

In a fourth aspect, the invention provides a method for editing a target adenosine present in an endogenous target RNA molecule in a cell in a subject, comprising the steps of: (i) administering to said subject a triterpene glycoside; and (ii) administering to said subject an EON or a HEON, wherein the EON after administration, can form a double-stranded complex with a region of the endogenous target RNA molecule comprising the target adenosine in the cell, and wherein the double-stranded complex can recruit an endogenous ADAR enzyme to deaminate the target adenosine into an inosine. Preferably, the EON and the triterpene glycoside are conjugated to each other and are thereby administered simultaneously. Preferably, the triterpene glycoside is AG1856.

In a fifth aspect, the invention provides a method for the deamination of a target adenosine in a target RNA molecule, preferably a pre-mRNA or mRNA molecule, in a cell, the method comprising the steps of: (i) providing the cell with a triterpene glycoside; (ii) providing the cell with an EON that can form a double-stranded complex with the target RNA molecule, or a region thereof, wherein the region comprises the target adenosine; (iii) allowing uptake by the cell of the EON; (iv) allowing annealing of the EON to the target RNA molecule; (v) allowing an endogenous ADAR enzyme to deaminate the target adenosine in the target RNA molecule to an inosine; and optionally (vi) identifying the presence of the inosine in the target RNA molecule. Preferably, the EON and the triterpene glycoside are conjugated to each other and are thereby provided to the cell simultaneously. Preferably, the triterpene glycoside is AG1856.

In a sixth aspect, the invention provides a method of treating a disease in a human subject in need thereof, the method comprising administering to the human subject a therapeutically effective amount of an EON and a triterpene glycoside. Preferably, the EON and the triterpene glycoside are conjugated to each other and are thereby administered simultaneously. Preferably, the triterpene glycoside is AG1856. Preferably, the disease is a cardiovascular disease, a disease involving the liver, a disease involving the kidney, a disease involving the pancreas, or a disorder of the central nervous system.

### BRIEF DESCRIPTION OF THE DRAWINGS

One or more embodiments of the invention will now be described, by way of example only, with reference to the accompanying drawings, in which:

FIG. 1 shows the percentage editing on a human APP target RNA transcript in ARPE-19 cells after administration of triterpene glycoside AG1856 and the subsequent administration of an RNA editing oligonucleotide (EON).

FIG. 2 shows the percentage editing in FIG. 2A liver, FIG. 2B kidney and FIG. 2C pancreas in mice that were treated with AG1856 and a subsequent administration of an EON (RM3835) targeting the mouse APP transcript. The fold change in editing over administration of the EON alone is given on the right y-axis.

FIG. 3 shows sequences (5′ to 3′) of EONs designed for editing the B4GALT1 transcript, with their respective SEQ ID NO's as shown. RM4838/EON13 is also referred to as B4GALT1-13. RM4830/EON05 is also referred to as B4GALT1-05. The chemical modifications of the EONs are as follows: m5Ue is 2′-MOE modified 5-methyl-uridine (similar to a 2′-MOE modified thymidine, or Te); m5Ce is 2′-MOE modified 5-methyl-cytidine; Ae and Ge are 2′-MOE modified adenosine and guanosine, respectively; Gm, Am, Um, and Cm are 2′-OMe modified guanosine, adenosine, uridine, and cytidine, respectively; Af, Uf, Gf, and Cf are 2′-F modified adenosine, uridine, guanosine, and cytosine, respectively; Zd (at the orphan nucleotide position) is a cytidine analog that is also referred to as a nucleoside carrying a Benner's base (as further outlined herein), with a deoxy moiety (=DNA) at the 2′ ribose position; C2f (at the orphan nucleotide position) is a 2′,2′-difluoro modified cytidine; m5Ud (or simply referred to as Ud) is a deoxynucleotide with a 5-methyl-uridine base; Cd (at the orphan nucleotide position) is deoxycytidine; an asterisk * refers to a PS linkage; a “!” refers to a PNdmi linkage; and a “\hat{ }” refers to a MP linkage. All other linkages are phosphodiester linkages.

FIG. 4 shows the percentage editing of endogenous B4GALT1 transcripts in human HepG2 cells after treatment with the indicated EONs in two different concentrations (1 and 5 μM) and saponin (AG1856). Negative controls were AG1856 only and non-treated cells.

FIG. 5 shows the percentage editing of endogenous B4GALT1 transcripts in liver spheroids generated from primary human hepatocytes, after treatment with the four indicated EONs+AG1856 (here abbreviated to AG).

FIG. 6 shows a set of EONs (SEQ ID NO:46 to 90, as indicated) designed with the addition of a GalNAc moiety attached to the 5′ terminus of the oligonucleotide. The chemical modifications are as provided in FIG. 3. L001 is a tri-antennary GalNAc moiety (OP-042; Hongene Biotech). L103 is a TEG linker linking the GalNAc moiety to the first nucleotide on the 5′ terminus.

FIG. 7 shows the editing percentage of the human B4GALT1 target transcript in primary human hepatocytes (PHH) after treatment with 5 μM EON in the presence of 1 μM saponin (AG1856), using the EONs of FIG. 6 together with EON01 and EON05 (see FIG. 3). A non-treated (NT) sample was taken as negative control.

FIG. 8 shows the editing percentage of the human B4GALT1 target transcript in PHH after treatment with 5 μM EON in the absence of any saponin, hence through gymnotic uptake, using the EONs of FIG. 7 together with EON01 and EON05 (see FIG. 3), exactly as outlined in FIG. 7. A non-treated (NT) sample was taken as negative control.

FIG. 9 shows on top the human ANGPTL3 target RNA sequence (5′ to 3′; SEQ ID NO: 121) with the target adenosine in bold face. Below the target sequence, the sequences (also 5′ to 3′) are given of the 30 EONs (RM5035 to RM5064 are SEQ ID NO:91 to 120, respectively) that were designed for editing the target adenosine. The chemical modifications in the EONs are as follows: t, a, g=2′-MOE modified thymidine, adenosine, guanosine, respectively; c=2′-MOE modified 5-methyl-cytosine; mU, mA, mG, mC=2′-OMe modified uridine, adenosine, guanosine, cytosine, respectively; fU, fA, fG, fC=2′-F modified uridine, adenosine, guanosine, cytosine, respectively; dA=deoxyadenosine; dZ=deoxycytidine analog carrying a Benner's base; “!”=PNdmi linkage; “*”=phosphorothioate linkage; (MeP)=methylphosphonate linkage.

FIG. 10 shows the percentage A to I editing of endogenous ANGPTL3 transcripts in human Huh-7 cells after gymnotic (no saponin) exposure to 30 EONs as indicated. A non-treated incubation was taken along as negative control (mock).

FIG. 11 shows the percentage A to I editing of endogenous ANGPTL3 transcripts in human Huh-7 cells after exposure to 30 EONs as in FIG. 10, but with the addition of 1 μM AG1856 to the culture medium. A non-treated incubation and an incubation with AG1856 were taken along as negative controls (AG1856 only and NT).

FIG. 12 shows the percentage A to I editing of endogenous ANGPTL3 transcripts in human primary hepatocytes that were grown into liver spheroids, after exposure to the 30 EONs as in FIG. 10 and FIG. 11, with the addition of 5 μM AG1856 to the culture medium. Two negative controls were taken along (mock and NT).

FIG. 13 shows the results of an identical experiments as in FIG. 12, but in which spheroids were incubated with 5 μM EON, using only RM5059 (EON #25), RM5060 (EON #26), RM5061 (EON #27), RM5062 (EON #28), RM5063 (EON #29), and RM5064 (EON #30), in the absence of the saponin.

FIG. 14 shows the editing percentage of an adenosine in a mouse Actin B target RNA transcript, in the liver (FIG. 14A), kidney (FIG. 14B), and spleen (FIG. 14C) of mice treated with PBS, PBS+AG1856, RM3891 EON, or RM3891+AG1856.

FIG. 15 shows an exemplary conjugate of AG1856 and an EON, linked by an ECMH moiety and a C6S linker, wherein the conjugation is at the 5′ terminus of the EON.

FIG. 16 shows the percentage editing of mApp transcripts in primary mouse hepatocytes after administration of EON alone (RM3835 or RM5522), a conjugate composition in which RM5522 is bound to AG1856 (RM5522@AG1856, as shown in FIG. 15) and a co-administration of RM5522 and AG1856 (not conjugated; shown by the “+”). The 53% purity of the RM5522@AG1856 conjugate is given between brackets. A non-treated (NT) sample served as a control. The significance increase between the different bars is provided by asterisks. The left y-axis shows the editing percentage, and the right y-axis shows the fold change in comparison to the treatment with RM5522 alone.

### DETAILED DESCRIPTION

As discussed above, a variety of different saponins have been used for many types of applications. Therapeutic uses of saponins have also been described (Weng A et al. 2009. Planta medica 75 (13): 1421-1422; Weng A et al. 2010. J Chromatography B 878 (7): 713-718; Weng A et al. 2012. Molecular Oncology 6 (3): 323-332; Weng A et al. J Controlled Disease 164 (1): 74-86; Thakur et al. 2014. J Chromatography B 955:1-9; Jia et al. 1998. J Natural Products 61 (11): 1368-1373; Haddad et al. 2004. Helvetica chimica acta 87 (1): 73-81; Fu et al. J Natural Products 68 (5): 754-758; Moniuszko-Szajwaj et al. 2016. Helvetica chimica acta 99 (5): 347-354; Fuchs H et al. 2017. Biomedicines 5 (2): 14). It has also been described that a specific saponin (SO1861) derived from Saponaria officinalis can mediate an improved intracellular delivery of peptide and lipid nanoparticles, as well as nucleic acids (Weng A et al. 2015. J Controlled Release 206:75-90; Sama S et al. 2017. Int J Pharmaceutics 534:195-205). WO2019/011914 discloses a saponin (GE1741) isolated from Gypsophila elegans rendering improved effects regarding the delivery of small molecules, such as nucleic acid molecules to cells (see also Sama S et al. 2018. J Biotechnology 284:131-139). WO2021/122998 (and EP3838910B1, accruing from the priority application) discloses yet another class of saponins, derived from Agrostemma githago L. with further improved properties over the earlier described SO1861 and GE1741 saponins, especially regarding toxicity and endosomal escape (see also Clochard J et al. 2020. Int J Pharm 589:119822).

The inventors of the present invention show here that the saponins (hereinafter also referred to as triterpene glycosides, or triterpene saponins) disclosed in WO2021/122998 display an unexpected, improved RNA editing efficiency produced by a particular kind of nucleic acid molecules, namely RNA editing oligonucleotides (EONs) in in vitro and in vivo settings, using a variety of administration set-ups. It was especially surprisingly found that when the triterpene glycoside was (bio) conjugated to the EON, that RNA editing was not hampered, but in fact drastically higher than when the triterpene glycoside was administered separately from the EON. It was surprisingly found that the recruitment of the endogenous ADAR enzyme after the EON has hybridized to the target molecule, apparently was not hampered by the presence of the saponin, and/or that trafficking through the cell towards the target transcript molecule was also not hampered by the attachment of the saponin to the EON. In an embodiment, the invention relates to the use of triterpene glycoside with a stereoisomeric form according to formula (I):

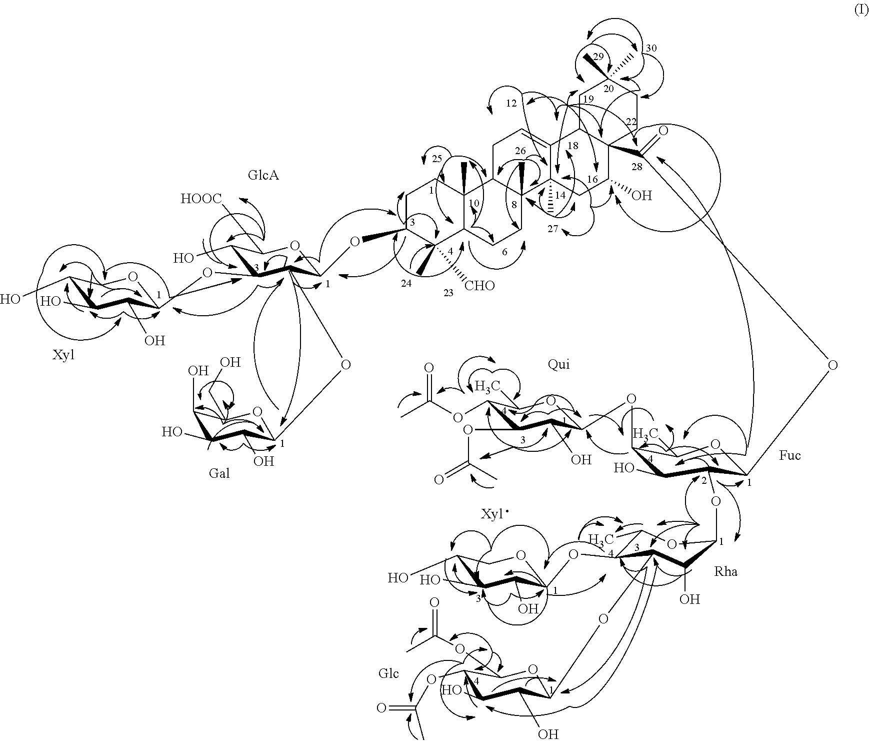

wherein the sugar groups are as follows: Fuc=fucose, Rha=Rhamnose, Gal=Galactose, Xyl=Xylose, Glc=Glucose, GlcA=Glucuronic acid, and Qui=Quinovose. This triterpene glycoside (or triterpene saponin, or simply ‘saponin’) is generally referred to herein as AG1856. It exhibits a structure motif known from for example GE1741, namely a double acetylated quinovose residue connected to a fucose residue which is, in turn, directly connected (via an ester bridge) to the C-28-chain of the quillac acid/gypsogenin. While in case of GE1741 a linear sugar chain is connected to the fucose residue, AG1856 bears a branched rhamnose residue carrying a xylose residue and an acetylated glucose residue.

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are adenosine (A) to inosine (I) conversions and cytidine (C) to uridine (U) conversions, which occur through enzymes called Adenosine Deaminases acting on RNA (ADAR) and APOBEC/AID (cytidine deaminases that act on RNA), respectively.

ADAR is a multi-domain protein, comprising a catalytic domain, and two to three double-stranded RNA recognition domains, depending on the enzyme in question. Each recognition domain recognizes a specific double stranded RNA (dsRNA) sequence and/or conformation. The catalytic domain does also play a role in recognizing and binding a part of the dsRNA helix, although the key function of the catalytic domain is to convert an A into I in a nearby, predefined, position in the target RNA, by deamination of the nucleobase. Inosine is read as guanosine by the translational machinery of the cell, meaning that, if an edited adenosine is in a coding region of an mRNA or pre-mRNA, it can recode the protein sequence. A to I conversions may also occur in 5′ non-coding sequences of a target mRNA, creating new translational start sites upstream of the original start site, which gives rise to N-terminally extended proteins, or in the 3′ UTR or other non-coding parts of the transcript, which may affect the processing and/or stability of the RNA. In addition, A to I conversions may take place in splice elements in introns or exons in pre-mRNAs, thereby altering the pattern of splicing. As a result, exons may be included or skipped. The enzymes catalysing adenosine deamination are within an enzyme family of ADARs, which include human deaminases hADAR1 and hADAR2, as well as hADAR3. However, for hADAR3 no deaminase activity has been demonstrated.

The use of oligonucleotides to edit a target RNA applying adenosine deaminase has been described (e.g., Woolf et al. 1995. PNAS 92:8298-8302; Montiel-Gonzalez et al. PNAS 2013, 110 (45): 18285-18290; Vogel et al. 2014. Angewandte Chemie Int Ed 53:6267-6271). A disadvantage of the method described by Montiel-Gonzalez et al. (2013) is the need for a fusion protein consisting of the boxB recognition domain of bacteriophage lambda N-protein, genetically fused to the adenosine deaminase domain of a truncated natural ADAR protein. It requires target cells to be either transduced with the fusion protein, which is a major hurdle, or that target cells are transfected with a nucleic acid construct encoding the engineered adenosine deaminase fusion protein for expression. The system described by Vogel et al. (2014) suffers from similar drawbacks, in that it is not clear how to apply the system without having to genetically modify the ADAR first and subsequently transfect or transform the cells harboring the target RNA, to provide the cells with this genetically engineered protein. U.S. Pat. No. 9,650,627 describes a similar system. The oligonucleotides of Woolf et al. (1995) that were 100% complementary to the target RNA sequences suffered from severe lack of specificity: nearly all adenosines in the target RNA strand that was complementary to the antisense oligonucleotide were edited.

It is known that ADAR may act on any dsRNA. Through a process sometimes referred to as ‘promiscuous editing’, the enzyme will edit multiple A's in the dsRNA. Hence, there was a need for methods and means that circumvent such promiscuous editing and only target specific adenosines in a target RNA molecule to become therapeutic applicable. Vogel et al. (2014) showed that such off-target editing can be suppressed by using 2′-O-methyl(2′-OMe) modified nucleosides in the oligonucleotide at positions opposite to adenosines that should not be edited and used a non-modified nucleoside directly opposite to the specifically targeted adenosine on the target RNA. However, the specific editing effect at the target nucleotide has not been shown to take place without the use of recombinant ADAR enzymes having covalent bonds with the AON. Several publications have now shown that the recruitment of endogenous ADAR (hence without the need for an exogenous and/or recombinant source) is feasible while maintaining a specificity in which a single adenosine within a target RNA molecule can be targeted and deaminated to an inosine. WO2016/097212 discloses antisense oligonucleotides (AONs) for the targeted editing of RNA, wherein the AONs are characterized by a sequence that is complementary to a target RNA sequence (therein referred to as the ‘targeting portion’) and by the presence of a stem-loop/hairpin structure (therein referred to as the ‘recruitment portion’), which is preferably non-complementary to the target RNA. Such oligonucleotides are referred to as ‘self-looping AONs’. The recruitment portion acts in recruiting a natural ADAR enzyme present in the cell to the dsRNA formed by hybridization of the target sequence with the targeting portion. Due to the recruitment portion, there is no need for conjugated entities or presence of modified recombinant ADAR enzymes. WO2016/097212 describes the recruitment portion as being a stem-loop structure mimicking either a natural substrate (e.g., the GluB receptor) or a Z-DNA structure known to be recognized by the dsRNA binding domains, or Z-DNA binding domains, of ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion as described is an intramolecular stem-loop structure, formed within the AON itself, and are thought to attract (endogenous) ADAR. Similar stem-loop structure-comprising systems for RNA editing have been described in WO2017/050306, WO2020/001793, WO2017/010556, WO2020/246560, and WO2022/078995.

WO2017/220751 and WO2018/041973 describe a next generation type of AONs that do not comprise such a stem-loop structure but that are (almost fully) complementary to the targeted area. In one aspect, one or more mismatching nucleotides, wobbles, or bulges exist between the oligonucleotide and the target sequence. A sole mismatch may be at the site of the nucleoside opposite the target adenosine, but in other embodiments AONs (or RNA editing oligonucleotides, abbreviated to ‘EONs’) were described with multiple bulges and/or wobbles when attached to the target sequence area. It appeared possible to achieve in vitro, ex vivo and in vivo RNA editing with EONs lacking a stem-loop structure that was not complementary to the target sequence and with endogenous ADAR enzymes when the sequence of the EON was carefully selected such that it could attract/recruit ADAR. The ‘orphan nucleoside’ or ‘orphan nucleotide’, which is defined as the nucleoside/nucleotide in the EON that is positioned directly opposite the target adenosine in the target RNA molecule, did not carry a 2′-OMe modification. The orphan nucleoside can be a deoxyribonucleoside (DNA), wherein the remainder of the EON could still carry 2′-O-alkyl modifications at the sugar entity (such as 2′-OMe), or the nucleotides directly surrounding the orphan nucleoside contained chemical modifications (such as DNA in comparison to RNA) that further improved the RNA editing efficiency and/or increased the resistance against nucleases. Such effects could even be further improved by using sense oligonucleotides (SONs) that ‘protected’ the EONs against breakdown (described in WO2018/134301). The use of chemical modifications and particular structures in oligonucleotides that could be used in ADAR-mediated editing of specific adenosines in a target RNA have been the subject of numerous publications in the field, such as WO2019/111957, WO2019/158475, WO2020/165077, WO2020/201406, WO2020/211780, WO2021/008447, WO2021/020550, WO2021/060527, WO2021/117729, WO2021/136408, WO2021/182474, WO2021/216853, WO2021/242778, WO2021/242870, WO2021/242889, WO2022/007803, WO2022/018207, WO2022/026928, and WO2022/124345. The use of specific sugar moieties has been disclosed in for instance WO2020/154342, WO2020/154343, WO2020/154344, WO2022/103839, and WO2022/103852, whereas the use of stereo-defined linker moieties (in general for oligonucleotides that for instance can be used for exon skipping, in gapmers, in siRNA, or specifically for RNA-editing oligonucleotides, related to a wide variety of target sequences) has been described in WO2011/005761, WO2014/010250, WO2014/012081, WO2015/107425, WO2017/015575 (HTT), WO2017/062862, WO2017/160741, WO2017/192664, WO2017/192679 (DMD), WO2017/198775, WO2017/210647, WO2018/067973, WO2018/098264, WO2018/223056 (PNPLA3), WO2018/223073 (APOC3), WO2018/223081 (PNPLA3), WO2018/237194, WO2019/032607 (C9orf72), WO2019/055951, WO2019/075357 (SMA/ALS), WO2019/200185 (DM1), WO2019/217784 (DM1), WO2019/219581, WO2020/118246 (DM1), WO2020/160336 (HTT), WO2020/191252, WO2020/196662, WO2020/219981 (USH2A), WO2020/219983 (RHO), WO2020/227691 (C9orf72), WO2021/071788 (C9orf72), WO2021/071858, WO2021/178237 (MAPT), WO2021/234459, WO2021/237223, and WO2022/099159. Next to these disclosures, an extensive number of publications relate to the targeting of specific RNA target molecules, or specific adenosines within such RNA target molecules, be it to repair a mutation that resulted in a premature stop codon, or other mutation causing disease. Examples of such disclosures in which adenosines are targeted within specified target RNA molecules are WO2020/157008 and WO2021/136404 (USH2A); WO2021/113270 (APP); WO2021/113390 (CMT1A); WO2021/209010 (IDUA, Hurler syndrome); WO2021/231673 and WO2021/242903 (LRRK2); WO2021/231675 (ASS1); WO2021/231679 (GJB2); WO2019/071274 and WO2021/231680 (MECP2); WO2021/231685 and WO2021/231692 (OTOF, autosomal recessive non-syndromic hearing loss); WO2021/231691 (XLRS); WO2021/231698 (argininosuccinate lyase deficiency); WO2021/130313 and WO2021/231830 (ABCA4); and WO2021/243023 (SERPINA1).

In a first aspect, the invention provides a composition comprising a triterpene glycoside and an RNA editing producing EON, wherein the EON can form a double-stranded complex with a region of a target RNA molecule in a cell, wherein the region of the target RNA molecule comprises a target adenosine, and wherein the double-stranded complex can recruit an endogenous ADAR enzyme to deaminate the target adenosine into an inosine, thereby editing the target RNA molecule. Hence, the EON itself, even though it is sometimes referred to as an RNA editing oligonucleotide, does not deaminate the target adenosine itself, but it can cause (generate, trigger, produce) the deamination of the target adenosine by hybridizing, binding to the target RNA molecule, or the complementary region thereof including the target adenosine, and by producing this double-stranded entity, a deaminating enzyme can be recruited that can subsequently deaminate the target adenosine to an inosine. A preferred triterpene glycoside that is used in all aspects of the invention is AG1856, that can be purified from seeds of Agrostemma githago L. A preferred triterpene glycoside that is used in all aspects of the invention is shown in formula (I). To increase cell entry efficiency and/or stability of the EON, it is—while in the composition—preferably hybridized to a fully or partially complementary sense oligonucleotide to form a heteroduplex RNA editing producing oligonucleotide (HEON) complex. Preferred HEON complexes are described in GB 2215614.5, not published). To increase the editing efficiency even further, it is preferred that the triterpene glycoside is bound to the EON, through conjugation. Preferably, the triterpene glycoside is attached to the 5′ terminus of the EON, although it is not excluded that the triterpene glycoside can also be bound to the 3′ terminus. The skilled person knows the manufacturing possibilities to conjugate the saponin to either the 5′ or the 3′ terminus, and the preferred linkers required for proper conjugation. In one embodiment, the nucleotide in the EON that is opposite the target adenosine is a cytidine, a cytidine analog, a cytidine derivative, a uridine, a uridine analog, or a uridine derivative. In one embodiment, at least one nucleotide in the EON comprises one or more non-naturally occurring chemical modifications in the ribose, the linkage, or the base moiety, with the proviso that the nucleotide in the EON that is opposite the target adenosine is not a cytidine comprising a 2′-OMe ribose substitution. In all aspects of the invention, the target RNA molecule is preferably pre-mRNA or mRNA. However, RNA editing according to the invention in any aspect, may also be applied to tRNA, rRNA, and vRNA, if needed. In one embodiment, the endogenous ADAR enzyme is human ADAR1, ADAR2 or ADAT. In one embodiment, the composition according to the invention is for use in the treatment of a cardiovascular disease, a disease involving the liver, a disease involving the kidney, a disease involving the pancreas, or a disorder of the central nervous system. A wide variety of diseases exist, that may be caused by many different genetic alterations that may not particularly affect only one sort of tissue. This means that the invention also relates to targeting target RNA molecules that are involved in diseases that may have a detrimental effect on several tissues. In another aspect, the invention also relates to deamination of a target adenosine that is in a wild-type transcript molecule, but wherein the deamination of the target adenosine results in a translated product that diminishes or ameliorates or prevents certain diseases or disorders. These include the introduction of loss-of-function alterations and gain-of-function alterations, for instance by removing or introducing a phosphorylation or glycosylation site, etc. In one embodiment, the target RNA molecule is endogenously present in the cell and the target RNA molecule is transcribed from a human gene selected from the group consisting of: SERPINA1, IDUA, HFE, ABCA4, USH2A, PCSK9, B4GALT1, NTCP, ALDH2, HTT, DMD, PNPLA3, APOC3, C9orf72, DMPK, RHO, MAPT, OTOF, SMN1, ASL, APP, PMP22, LRRK2, ASS1, GJB2, MECP2, and RS1.

In a second aspect the invention provides a kit-of-parts comprising (i) a first pharmaceutical composition comprising a triterpene glycoside; and (ii) a second pharmaceutical composition comprising an EON, wherein the EON can form a double-stranded complex with a region of a target RNA molecule in a cell, wherein the region of the target RNA molecule comprises a target adenosine, and wherein the double-stranded complex can recruit an endogenous ADAR enzyme to deaminate the target adenosine into an inosine, thereby editing the target RNA molecule. The triterpene glycoside and the EON may be administered separately in a dosing regimen, for example wherein the triterpene glycoside is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days before or after the EON is administered, or wherein the triterpene glycoside is administered 1, 2, 3, 4, 5, 6, 7, 8, 9, 10, 11, or 12 weeks before or after the EON is administered, or even several months apart. Notably the triterpene glycoside and the EON may also be administered on the same day, but separately in separate administrations, instantly following each other or wherein the administrations are separated, for example by 1, 2, 3, 4, 5, 6, 7, or 8 hours. In a preferred embodiment, the triterpene glycoside and the EON are administered simultaneously, preferably such that the two entities are conjugated to each other, which means that the kit-of-parts is such that the EON-saponin forms a single part. In one embodiment, the triterpene glycoside that is in the kit-of-parts is AG1856. In one embodiment, the EON in the second pharmaceutical composition is hybridized to a fully or partially complementary sense oligonucleotide to form a HEON. The HEON may also be separate in the kit-of-parts and be hybridized to the EON (or the EON-saponin conjugate) briefly before administration. In one embodiment, the nucleotide in the EON that is opposite the target adenosine is a cytidine, a cytidine analog, a cytidine derivative, a uridine, a uridine analog, or a uridine derivative. In one embodiment, at least one nucleotide in the EON comprises one or more non-naturally occurring chemical modifications in the ribose, the linkage, or the base moiety, with the proviso that the nucleotide in the EON that is opposite the target adenosine, is not a cytidine comprising a 2′-OMe ribose substitution. In one embodiment, the target RNA molecule is pre-mRNA or mRNA. In one embodiment, the endogenous ADAR enzyme is human ADAR1, ADAR2 or ADAT. In one embodiment the kit-of-parts is for use in the treatment of a cardiovascular disease, a disease involving the liver, a disease involving the kidney, a disease involving the pancreas, or a disorder of the central nervous system. In one embodiment, the target RNA molecule is endogenously present in the cell and wherein the target RNA molecule is transcribed from a human gene selected from the group consisting of: SERPINA1, IDUA, HFE, ABCA4, USH2A, PCSK9, B4GALT1, NTCP, ALDH2, HTT, DMD, PNPLA3, APOC3, C9orf72, DMPK, RHO, MAPT, OTOF, SMN1, ASL, APP, PMP22, LRRK2, ASS1, GJB2, MECP2, and RS1.

In a third aspect, the invention provides a method for editing a target adenosine present in an endogenous target RNA molecule in a cell in a subject, comprising the steps of: (i) administering to said subject a triterpene glycoside, preferably AG1856; and administering to said subject an EON or a HEON, wherein the EON after administration, can form a double-stranded complex with a region of the endogenous target RNA molecule comprising the target adenosine in the cell, and wherein the double-stranded complex can recruit an endogenous ADAR enzyme to deaminate the target adenosine into an inosine. Preferably, the EON and the triterpene glycoside and the EON are administered simultaneously, preferably as an EON-triterpene glycoside conjugate composition. In one embodiment, the cell is a liver cell (preferably a hepatocyte), a kidney cell, or a neuron (preferably a neuronal cell of the CNS). In one embodiment the EON-triterpene glycoside conjugate is for delivery to a liver cell and the EON is also conjugated to a GalNAc moiety at the opposite side of the triterpene glycoside.

In a fourth aspect the invention provides a method for the deamination of a target adenosine in a target RNA molecule, preferably a pre-mRNA or mRNA molecule, in a cell, the method comprising the steps of: (i) providing the cell with a triterpene glycoside, preferably AG1856; (ii) providing the cell with an EON that can form a double-stranded complex with the target RNA molecule, or a region thereof, wherein the region comprises the target adenosine; (iii) allowing uptake by the cell of the EON; (iv) allowing annealing of the EON to the target RNA molecule; (v) allowing an endogenous ADAR enzyme to deaminate the target adenosine in the target RNA molecule to an inosine; and optionally (vi) identifying the presence of the inosine in the target RNA molecule. The triterpene glycoside does not necessarily need to be provided before the EON is provided; these steps (i) and (ii) may also be reversed, or take place simultaneously, for instance when the EON and the triterpene glycoside are in a single composition, or when they have been conjugated to each other. In one embodiment, the EON is hybridized to a fully or partially complementary sense oligonucleotide to form a HEON before the EON forms the double-stranded complex with the target RNA molecule. The HEON may also be in the same composition as the triterpene glycoside. In yet another embodiment, the saponin is conjugated to the sense strand, which is hybridized to the EON through Watson-Crick base pairing.

In a fifth aspect the invention provides a method of treating a disease in a human subject in need thereof, the method comprising administering to the human subject a therapeutically effective amount of an EON and a triterpene glycoside, wherein the triterpene glycoside is preferably AG1856. Preferred disease that are treated with the composition of the invention (or the kit-of-parts, when the EON and triterpene glycoside are administered separately) is a cardiovascular disease, a disease involving the liver, a disease involving the kidney, a disease involving the pancreas, or a disorder of the central nervous system. However, other disorders are not explicitly excluded, whereas the skilled person is aware that when a particular disease is caused by a genetic aberration that affects multiple organs and/or tissues, an administration of the EON and the triterpene glycoside according to the invention may treat a variety of organs and/or tissues.

Although in a preferred embodiment the EON of the composition of the present invention is administered as a single-stranded oligonucleotide comprising an orphan nucleotide opposite the target adenosine, wherein the orphan nucleotide is chemically modified as disclosed herein, and wherein the remainder of the oligonucleotide is chemically modified to prevent it from nuclease breakdown also as disclosed herein, in another embodiment, the invention relates to any kind of oligonucleotide or heteroduplex oligonucleotide complex, that may or may not be bound to hairpin structures (internally or at the terminal end(s)), that may be bound to ADAR or catalytic domains thereof, or wherein the oligonucleotide is in a circular format. It is to be understood that any kind of oligonucleotide-based RNA editing is encompassed by the present invention if it relates to the deamination of a nucleotide while using a triterpene glycoside (or triterpene saponin), preferably AG1856, for delivery. In a preferred aspect, the EON of the present invention is a ‘naked’ oligonucleotide (in the sense that it is not delivered through vector ‘means’ such as viruses or plasmids that encode the EON), comprising a variety of chemical modifications in the ribose sugar, the base, and/or the internucleoside linkage of one or more of the nucleotides within the sequence, and can recruit endogenous ADAR for the deamination of the target adenosine.

In another preferred embodiment, the EON of the composition of the invention is bound (hybridized) to a sense oligonucleotide to form a heteroduplex RNA editing oligonucleotide complex, or HEON in short, wherein the sense oligonucleotide may be of the same length, shorter or longer than the antisense oligonucleotide, and wherein the sense strand may be completely complementary to the antisense oligonucleotide or partially complementary. Partial complementary antisense and sense oligonucleotides in such a complex are sometimes also referred to as partial HEONs, or pHEONs (see GB 2215614.5, not published). In an embodiment the triterpene glycoside that is either administered separately or together (preferably conjugated) with the antisense oligonucleotide can therefore also, in another embodiment, be combined with a HEON or a pHEON.

In an embodiment, the triterpene glycoside is bound, or conjugated to the EON or the sense strand in a HEON, either at the 5′ end or the 3′. Preferably, the saponin is conjugated to the 5′ terminus of the EON, preferably by using a linker. Conjugation can be achieved in a wide variety of manners, using a variety of linking moieties, generally known to the person skilled in the art using conjugation methods known to the person skilled in the art. Conjugation (also referred to as ‘bioconjugation’, since it relates to biomedicine) means that the two parts (the EON and the saponin) are bound to each other before, and during administration, be it in vitro in cell cultures, or in vivo, during administration in mice, non-human primates, or in clinical trials, in humans. The conjugation between the EON and the saponin is generally made in a laboratory setting, or in a manufacturing facility, since the EON is made in a laboratory setting as well, while the saponin is generally purified from natural sources. The binding may be non-reversible, or reversible. It should be noted that when a targeting moiety, such as GalNAc is used, the GalNAc and the triterpene glycoside (but also the tocopherol or cholesterol, or analog thereof, as disclosed in GB 2215614.5) may all be bound to the EON, the sense strand, at the 5′ end, and/or at the 3′ end of each of the oligonucleotide strands. In a preferred embodiment, the saponin is conjugated to the 5′ terminus of the EON, while the GalNAc moiety is bound to the 3′ terminus of the EON when delivery to liver cells (especially hepatocytes) is desired. The skilled person is capable of binding each of such additional groups to any position within the EON and/or sense strand (when it is a HEON complex), based on the administrative route, the disease to be treated, the target sequence, etcetera.

The term ‘nucleoside’ refers to the nucleobase linked to the (deoxy) ribosyl sugar, without phosphate groups. A ‘nucleotide’ is composed of a nucleoside and one or more phosphate groups. The term ‘nucleotide’ thus refers to the respective nucleobase-(deoxy) ribosyl-phospholinker, as well as any chemical modifications of the ribose moiety or the phospho group. Thus, the term would include a nucleotide including a locked ribosyl moiety (comprising a 2′-4′ bridge, comprising a methylene group or any other group), an unlocked nucleic acid (UNA), a threose nucleic acid (TNA), a nucleotide including a linker comprising a phosphodiester, phosphonoacetate, phosphotriester, phosphorothioate (PS), phosphoro(di)thioate, methylphosphonate (MP), methyl thiophosphonate, phosphoramidate, phosphoryl guanidine linkages, and the like. Sometimes the terms adenosine and adenine, guanosine and guanine, cytidine and cytosine, uracil and uridine, thymine and thymidine/uridine, inosine and hypoxanthine, are used interchangeably to refer to the corresponding nucleobase on the one hand, and the nucleoside or nucleotide on the other. Thymine (T) is also known as 5-methyluracil (m⁵U) and is a uracil (U) derivative; thymine, 5-methyluracil and uracil can be interchanged throughout the document text. Likewise, thymidine is also known as 5-methyluridine and is a uridine derivative; thymidine, 5-methyluridine and uridine can be interchanged throughout the document text. Sometimes the terms nucleobase, nucleoside and nucleotide are used interchangeably, unless the context clearly requires differently, for instance when a nucleoside is linked to a neighbouring nucleoside and the linkage between these nucleosides is modified. As stated herein, a nucleotide is a nucleoside plus one or more phosphate groups. The terms ‘ribonucleoside’ and ‘deoxyribonucleoside’, or ‘ribose’ and ‘deoxyribose’ are as used in the art.

The term ‘saponin’ has its regular scientific meaning and here refers to a group of amphipathic glycosides which comprise one or more hydrophilic glycone moieties combined with a lipophilic aglycone core which is a sapogenin. The saponin may be naturally occurring, derived from natural resources, generated synthetically, or non-naturally occurring. The term ‘saponin’ includes naturally occurring saponins, derivatives of natural-occurring saponins and saponins synthesized de novo through chemical and/or biotechnological synthesis routes.

The term ‘saponin derivative’ has its regular scientific meaning and here refers to a saponin, i.e., a modified saponin, which has a chemical modification at a position where previously an aldehyde group was present in the non-derivatized saponin before being subjected to chemical modification for provision of the saponin derivative. For examples, the saponin derivative is provided by chemical modification of an aldehyde group, in a saponin upon which the saponin derivative is based, i.e., the saponin is provided and an aldehyde group is chemically modified therewith providing the saponin derivative. For example, the saponin that is derivatized for provision of the saponin derivative is a naturally occurring saponin. Typically, the saponin derivative is a synthetic saponin, typically the saponin derivative is a derivatisation of a natural saponin, and is thus derived from a natural saponin, although a saponin derivative can also be derived from a synthetic saponin which may or may not have a natural counterpart. Typically, the saponin derivative has not a natural counterpart, i.e., the saponin derivative is not produced naturally by for instance plants or trees. Optionally, the saponin derivative further has one or more chemical modifications at positions where previously any of a carboxyl group, carboxylic acid group, acetate group and/or an acetyl group was present in the non-derivatized or derivatized saponin before subjected to chemical modification for provision of the saponin derivative. For example, the saponin derivative is provided and an aldehyde group, a carboxyl group, a carboxylic acid group, an acetate group and/or an acetyl group is chemically modified therewith providing the saponin derivative.

The term ‘mono-desmosidic saponin’ has its regular scientific meaning and here refers to a triterpenoid or steroid or alkaloid saponin containing a single saccharide chain bound to the aglycone core, wherein the saccharide chain consists of one or more sugar moieties.

The term ‘bis-desmosidic saponin’ has its regular scientific meaning and here refers to a triterpenoid or steroid or alkaloid saponin containing two saccharide chains bound to the aglycone core, wherein each of the two saccharide chains consists of one or more sugar moieties.

The term ‘triterpenoid saponin’ has its regular scientific meaning and here refers to a saponin having a triterpenoid-type of aglycone core structure. The triterpenoid saponin differs from a saponin based on a steroid glycoside such as sapogenol in that such saponin comprising a steroid glycoside has a steroid core structure, and the triterpenoid saponin differs from a saponin bases on an alkaloid glycoside such as tomatidine in that such saponin comprising an alkaloid glycoside has an alkaloid core structure.

In an embodiment, a saponin derivative comprises an aglycone core structure selected from the group consisting of:
2alpha-hydroxy oleanolic acid;
16alpha-hydroxy oleanolic acid;
hederagenin (23-hydroxy oleanolic acid);
16alpha,23-digudroxy oleanolic acid;
gypsogenin;
quillaic acid;
protoaescigenin-21 (2-methylbut-2-enoate)-22-acetate;
23-oxo-barringtogenol C-21,22-bis(2-methylbut-2-enoate);
23-oxo-barringtogenol C-21 (2-methylbut-2-enoate)-16,22-diacetate;
digitogenin;
3,16,28-trihydroxy oleanan-12-en;
gypsogenic acid; and
derivatives thereof.

Preferably, the saponin derivative comprises an aglycone core structure selected from quillaic acid and gypsogenin or derivatives thereof, more preferably the saponin derivative aglycone core structure is quillaic acid or a derivative thereof.

A conjugate of EON and saponin can take many forms and structures, a key feature being at least one saponin and at least one EON (or the sense strand of a HEON) being covalently linked to one another. As such, general structures of conjugate compositions of the present disclosure can be depicted as:

X-Y-Z,
X-(Y)ₙ-Z,
X-Y(-Z)ₙ,
X-(Y-Z)ₙ,
(X)ₙ-Y-Z,
(X-Y)ₙ-Z,
(X)ₙ-Y-(Z)ₙ,
(X-Y)ₙ-(Z)ₙ,
(X)ₙ-(Y-Z)ₙ,
or
(X)ₙ-(Y)ₙ-(Z)ₙ

in which X refers to an EON connected to one or more linkers at the same or multiple sites, Y is one or more linker that can be of the same type, or in each instance, of a different type, Z is a saponin or a cluster of saponins (for example a dendrimer structure containing multiple saponins that is subsequently connected to the linker), and n=2-10. A conjugate composition comprising any one of such structures can thus be composed of a single EON and a single saponin, or multiple EONs and a single saponin, or multiple saponins and a single EON, or multiple EONs and multiple saponins. EONs and saponins are linked to each other by one or more linkers; a single saponin is understood to be linked to a single EON through one or more linkers, whereas multiple saponins may be linked together first as a cluster (such as a dendrimer) and then connected to the one or more linkers or connected to the same linker; the linker essentially acting as part of the cluster. An isolated linker has therefore at least two sites of reactivity to connect at least one EON (or another linker connecting eventually to at least one EON) to at least one saponin (or another linker connecting eventually to at least one a saponin).

A single linker can be classified as a stable or a labile linker. Stable linkers are understood to have a far longer metabolic, chemical, or biological stability than labile linkers and are not introduced with the intention of cleavage of the linker metabolically, chemically, or biologically. Examples of stable linkers are alkyl chains, amides, thioethers (e.g., thiol/ene), ‘click’ linkages introduced by e.g., azide/(constrained) alkyne or triazine/(constrained) alkyne reaction. Examples of labile linkers are those that are cleaved under certain pH (e.g., oxime, hydrazone, acetal, carbonate, silyl ether, semicarbazone), reducing environments (e.g., disulfide), or enzymatic conditions (e.g., phosphoester hydrolysis, cathepsin cleavage, endonuclease cleavage). Labile linkers may also further comprise self-immolative moieties. It is understood that the classification of a linker being stable of labile may differ for different objectives and associated timescales: whereas e.g., a 40-hour linker half-life may be classified as stable for applications in which linker cleavage is desired in the minute time range, it may be classified as labile for applications that desire a linker cleavage in the week time range.

A conjugate may contain more than a single linker connecting the (more than one) EON and the (more than one) saponin, and both linkers may be of the same or different types. For example, a conjugate may have the following structure: EON-linker1-linker2-saponin with linker1 being an C6 thiolinker and linker2 being an EMCH linker, shown in Formula (II):

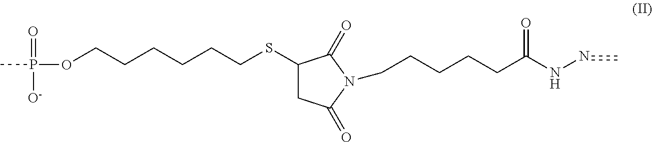

A linker links two chemical moieties together and thus possess at least two reactive groups to enable covalent attachment to these moieties. The moiety connecting these two or more reactive groups can be any chemical moiety as is known to those skilled in the art. For example, such connecting moiety can be a (branched) alkyl chain, or the diol, diamine, or disulfide thereof, polyethylene glycol, or triethylene glycol, or hexaethylene glycol.

To the person skilled in the art, many linkers and combinations thereof are known and can be applied to produce a conjugate of (at least one) EON with (at least one) saponin.

In a conjugate of (at least one) EON with (at least one) saponin, as disclosed herein, the linker connecting to the saponin may be covalently coupled to the saponin's aldehyde moiety, carboxylic acid moiety, or a primary alcohol or a secondary alcohol; or a mixture of these. Alternatively, the saponin may be chemically modified to allow coupling to a linker in another position in either one of the carbohydrate moieties (including carbon atoms and acetyl groups) or the aglycon (including carbon atoms, a double bond if present). The (at least one) saponin, thus connected to (at least one) linker, is connected to the (at least one) EON at e.g. its 3′ terminus, 5′ terminus, a 1′ position, a 2′ position, a 3′ position, a 4′ position, a 5′ position, the secondary amine in amino-LNA, at a nucleobase (e.g., at the 5 position of pyrimidine bases or N7 of purine bases), at a linkage (e.g., through a phosphoroamidate linkage, or phosphotriester) or through an unnatural nucleotide analogue such as an unlocked nucleic acid (UNA) monomer or other branching element in the EON allowing for attachment.

A conjugate composition of an EON and a saponin, as disclosed herein, may further contain other moieties that are covalently coupled through (one or more) stable or labile linkers as described above. Examples of such moieties are carbohydrates (e.g. GalNAc, Glc, GalN, GlcN or clusters thereof), lipids, vitamins, small molecules, drugs, peptides, antibodies) that may bestow additional tissue or cellular targeting, general or specific cellular uptake, endosomal escape, protein binding and/or intracellular trafficking characteristics to the conjugate.

In an embodiment, a kit-of-parts is provided in which a first formulation comprises an EON bound to a GalNAc moiety (for delivery to liver cells) and a second formulation comprises a triterpene glycoside bound to a GalNAc as well (also for delivery to liver cells). Both conjugates can then migrate to the liver where the triterpene glycoside can subsequently add to the entry and endosomal release of the EON that has also migrated to the liver cells because of its GalNAc conjugation.

A conjugate of an EON and a triterpene glycoside as disclosed herein may further be formulated as nanoparticles, such as lipid-, metal-, carbon-, ceramics-, or polymer-based nanoparticles.

A conjugate of an EON and a triterpene glycoside as disclosed herein may be used as a single active compound or may be combined in one composition with unconjugated EON to lower the total exposure of saponin, if needed.

The term “linker” has its regular scientific meaning, and linkers are commonly known in the art of bioconjugation. Here, the term linker refers to a chemical moiety or a linear stretch of amino-acid residues complexed through peptide bonds, which is suitable for covalently attaching (binding) a first molecule, such as a triterpene saponin as disclosed herein, to another molecule, such as an EON or an oligonucleotide of a (p)HEON as disclosed herein, or to a scaffold, for example composed of or comprising amino acid residues, nucleic acids, etc. Typically, the linker comprises a chain of atoms linked by chemical bonds. Any linker molecule or linker technology known in the art can be used in the compositions of the present disclosure. The linker is preferably a linker for covalently binding of molecules through a chemical group on such a molecule suitable for forming a covalent linkage or bond with the linker. Exemplary linkers are disclosed in WO2022/164316 which is a publication that also discloses multiple saponins and derivatives thereof that can be applied in combination with an EON or (p)HEON as disclosed herein. Preferred linkers are KMUH, EMCH, BMPH and Maleimide-Peg2-hydrazide.

Whenever reference is made to an oligonucleotide, oligo, ON, ASO, oligonucleotide composition, antisense oligonucleotide, AON, (RNA) editing oligonucleotide, EON, and RNA (antisense) oligonucleotide, both oligoribonucleotides and deoxyoligoribonucleotides are meant unless the context dictates otherwise. Potentially the oligonucleotide may completely lack RNA or DNA nucleotides (as they appear in nature) and may consist completely of modified nucleotides. Whenever reference is made to an ‘oligoribonucleotide’ it may comprise the bases A, G, C, U, or I. Whenever reference is made to a ‘deoxyoligoribonucleotide’ it may comprise the bases A, G, C, T, or I. However, an oligonucleotide of the present invention may comprise a mix of ribonucleosides and deoxyribonucleosides. When a deoxyribonucleotide is used, hence without a modification at the 2′ position of the sugar, the nucleotide is often abbreviated to dA. dC, dG or T in which the ‘d’ represents the deoxy nature of the nucleoside, while a ribonucleoside that is either normal RNA or modified at the 2′ position is often abbreviated without the ‘d’, and often abbreviated with their respective modifications and as explained herein.

Whenever reference is made to nucleotides in the oligonucleotide, such as cytosine, 5-methylcytosine, 5-hydroxymethylcytosine, 5-formylcytosine, 5-acetylcytosine, 5-hydroxycytosine, and β-D-glucosyl-5-hydroxymethylcytosine are included. Whenever reference is made to adenine, N6-methyladenine, 8-oxo-adenine, 2,6-diaminopurine and 7-methyladenine are included. Whenever reference is made to uracil, dihydrouracil, isouracil, N3-glycosylated uracil, pseudouracil, 5-methyluracil, N1-methylpseudouracil, 4-thiouracil and 5-hydroxymethyluracil are included. Whenever reference is made to guanine, 1-methylguanine, 7-methylguanosine, N2, N2-dimethylguanosine, N2, N2,7-trimethylguanosine and N2,7-dimethylguanosine are included. Whenever reference is made to nucleosides or nucleotides, ribofuranose derivatives, such as 2′-deoxy, 2′-hydroxy, and 2′-O-substituted variants, such as 2′-OMe, are included, as well as other modifications, including 2′-4′ bridged variants. Whenever reference is made to oligonucleotides, linkages between two mononucleotides may be phosphodiester linkages as well as modifications thereof, including, phosphonoacetate, phosphotriester, PS, phosphoro(di)thioate, MP, phosphoramidate linkers, phosphoryl guanidine, thiophosphoryl guanidine, sulfono phosphoramidate and the like.

The term ‘comprising’ encompasses ‘including’ as well as ‘consisting of’, e.g., a composition ‘comprising X’ may consist exclusively of X or may include something additional, e.g., X+Y. The term ‘about’ in relation to a numerical value x is optional and means, e.g., x+10%.

The word ‘substantially’ does not exclude ‘completely’, e.g., a composition which is ‘substantially free from Y’ may be completely free from Y. Where relevant, the word ‘substantially’ may be omitted from the definition of the invention.

The term ‘complementary’ as used herein refers to the fact that the EON hybridizes under physiological conditions to a second nucleic acid strand (for instance when the oligonucleotide as a first nucleic acid strand (=guide oligonucleotide) forms a heteroduplex RNA editing oligonucleotide complex, or HEON, with another complementary nucleic acid strand), or when it forms a double stranded complex with the target RNA sequence. The term does not necessarily mean that each nucleotide in a nucleic acid strand has a perfect pairing with its opposite nucleotide in the opposite sequence. In other words, while an EON may be complementary to a target sequence, there may be mismatches, wobbles and/or bulges between the oligonucleotide and the target sequence, while under physiological conditions that EON still hybridizes to the target sequence such that the cellular RNA editing enzymes can edit the target adenosine. The term ‘substantially complementary’ therefore also means that despite the presence of the mismatches, wobbles, and/or bulges, the EON has enough matching nucleotides between the EON and target sequence that under physiological conditions the EON hybridizes to the target RNA. As shown herein, an EON may be complementary, but may also comprise one or more mismatches, wobbles and/or bulges with the target sequence, if under physiological conditions the EON is able to hybridize to its target.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand.

References to ‘hybridisation’ typically refer to specific hybridisation and exclude non-specific hybridisation. Specific hybridisation can occur under experimental conditions chosen, using techniques well known in the art, to ensure that most stable interactions between probe and target are where the probe and target have at least 70%, preferably at least 80%, more preferably at least 90% sequence identity.

The term ‘mismatch’ is used herein to refer to opposing nucleotides in a double stranded RNA complex which do not form perfect base pairs according to the Watson-Crick base pairing rules. In the historical sense, mismatched nucleotides are G-A, C-A, U-C, A-A, G-G, C-C, U-U pairs. In some embodiments first nucleic acid strands of the present invention comprise fewer than four mismatches with the target sequence, for example 0, 1 or 2 mismatches. ‘Wobble’ base pairs are G-U, I-U, I-A, and I-C base pairs. Although a G: G pairing would be considered a mismatch, that does not necessarily mean that the interaction is unstable, which means that the term ‘mismatch’ may be somewhat outdated based on the current invention where a Hoogsteen base-pairing may be seen as a mismatch based on the origin of the nucleotide but still be relatively stable. An isolated G: G pairing in duplex RNA can for instance be quite stable, but still be defined as a mismatch.

The term ‘splice mutation’ relates to a mutation in a gene that encodes for a pre-mRNA, wherein the splicing machinery is dysfunctional in the sense that splicing of introns from exons is disturbed and due to the aberrant splicing, the subsequent translation is out of frame resulting in premature termination of the encoded protein. Often such shortened proteins are degraded rapidly and do not have any functional activity.

In one embodiment, the EON capable of forming a double stranded nucleic acid complex with a target RNA molecule, wherein the double stranded nucleic acid complex can recruit an adenosine deaminating enzyme for deamination of at least one target adenosine in the target RNA molecule comprises a cytidine analog that is directly opposite the at least one target adenosine, in which the cytidine analog serves as an H-bond donor at the N3 site. Preferably, the cytidine analog is pseudoisocytidine (piC), or Benner's base Z. These cytidine analog nucleotides can come in an RNA or DNA format, or potentially modified at the 2′ position. Other cytidine analogs that can also be used in oligonucleotides according to the invention are 5-hydroxyC-H+, 5-aminoC-H+ and 8-oxoA (syn), cytidine C5 methyl, ethyl, propyl, etc., variants of the Benner's base Z that have different substituents than nitro (e.g. alkyl, F, Cl, Br, CN, etc.) and variants of 8-oxoA that are substituted at C2 (methyl, ethyl, propyl, halogens, etc). In one embodiment, the cytidine or the cytidine analog does not carry a 2′-O-Me or 2′-MOE ribose modification.

In one embodiment, the EON that is capable of forming a double stranded nucleic acid complex with a target RNA molecule, wherein the double stranded nucleic acid complex can recruit an adenosine deaminating enzyme for deamination of at least one target adenosine in the target RNA molecule, comprises a uridine analog or uridine derivative that is directly opposite the target adenosine, wherein the uridine analog or uridine derivative serves as an H-bond donor at the N3 site. Examples of preferred uridine analogs and uridine derivatives are iso-uridine, pseudouridine, 4-thiouridine, thienouridine, 5-methoxyuridine, dihydrouridine, 5-methyluridine N3-glycosylated uridine, dihydro-iso-uridine N3-uracil, and N3-glycosylated uracil. These uridine analogs/derivatives can come in an RNA or DNA format or can potentially be modified at the 2′ position. Other uridine analogs that can also be used in oligonucleotides according to the invention are derivatives of iso-uridine, such as substituted iso-uridine variants (with e.g., nitro, alkyl, F, Cl, Br, CN, etc.).

An EON (and the complementary nucleic acid strand when two oligonucleotides form a HEON) used in the present invention may be chemically modified almost in its entirety, for example by providing nucleotides with a ribose sugar moiety carrying a 2′-OMe substitution, a 2′-F substitution, or a 2′-MOE substitution. The orphan nucleotide in the EON may comprise a diF modification at the 2′ position of the sugar, or comprises a deoxyribose (2′-H, DNA), and in yet a further embodiment, at least one and in another embodiment both the two neighbouring nucleotides flanking the orphan nucleotide do not comprise a 2′-OMe modification. Complete modification wherein all nucleotides of the oligonucleotide hold a 2′-OMe modification, with natural bases, results in a non-functional oligonucleotide as far as RNA editing goes (known in the art), presumably because it hinders the ADAR activity at the targeted position. In general, an adenosine in a target RNA can be protected from editing by providing an opposing nucleotide with a 2′-OMe group (at least when there are no other chemical substitutions or modifications within the nucleotide), or by providing a guanine or adenine as opposing base, as these two nucleobases are also able to reduce editing of the opposing adenosine.

Various chemistries and modifications are known in the field of oligonucleotides that can be readily used. The regular internucleosidic linkages between the nucleotides may be altered by mono- or di-thioation of the phosphodiester bonds to yield PS esters or phosphorodithioate esters, respectively. Other modifications of the internucleosidic linkages are possible, including amidation and peptide linkers.

In an embodiment, the EON of the present invention comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides.

It is known in the art that RNA editing entities (such as human ADAR enzymes) edit dsRNA structures with varying specificity, depending on several factors. One important factor is the degree of complementarity of the two strands making up the dsRNA sequence. Perfect complementarity of the two strands usually causes the catalytic domain of human ADAR to deaminate adenosines in a non-discriminative manner, reacting with any adenosine it encounters. The specificity of hADAR1 and 2 can be increased by introducing chemical modifications and/or ensuring several mismatches in the dsRNA, which presumably helps to position the dsRNA binding domains in a way that has not been clearly defined yet. Additionally, the deamination reaction itself can be enhanced by providing an oligonucleotide that comprises a mismatch opposite the adenosine to be edited. Those of skill in the art will be capable of designing the complementary portion of the oligonucleotide according to their needs.

It will be understood by a person having ordinary skill in the art that the extent to which the editing entities (such as the ADAR1 or ADAR2 enzyme) inside the cell are redirected to other target sites may be regulated by varying the affinity of the first nucleic acid strand for the recognition domain of the editing molecule. The exact modification may be determined through some trial and error and/or through computational methods based on structural interactions between the EON and the recognition domain of the editing molecule. In addition, or alternatively, the degree of recruiting and redirecting the editing entity resident in the cell may be regulated by the dosing and the dosing regimen of the EON. This is something to be determined by the experimenter (in vitro) or the clinician, usually in phase I and/or II clinical trials.

RNA editing molecules present in the cell will usually be proteinaceous in nature, such as the ADAR enzymes found in metazoans, including mammals. Preferably, the cellular editing entity is an enzyme, more preferably an adenosine deaminase or a cytidine deaminase, still more preferably an adenosine deaminase. These are enzymes with ADAR activity. The ones of most interest are the human ADARs, hADAR1 and hADAR2, including any isoforms thereof. RNA editing enzymes known in the art, for which oligonucleotide constructs according to the invention may conveniently be designed, include the adenosine deaminases acting on RNA (ADARs), such as hADAR1 and hADAR2 in humans or human cells and cytidine deaminases. It is known that hADAR1 exists in two isoforms; a long 150 kDa interferon inducible version and a shorter, 110 kDa version, that is produced through alternative splicing from a common pre-mRNA. Consequently, the level of the 150 kDa isoform available in the cell may be influenced by interferon, particularly interferon-gamma (IFN-γ). hADAR1 is also inducible by TNF-α. This provides an opportunity to develop combination therapy, whereby IFN-γ or TNF-α and EONs according to the invention are administered to a patient either as a combination product, or as separate products, either simultaneously or subsequently, in any order. Certain disease conditions may already coincide with increased IFN-γ or TNF-α levels in certain tissues of a patient, creating further opportunities to make editing more specific for diseased tissues. It will be understood by a person having ordinary skill in the art that the extent to which the editing entities inside the cell are redirected to other target sites may be regulated by varying the affinity of the first nucleic acid strand for the recognition domain of the editing molecule.

The invention concerns the modification of target RNA sequences in eukaryotic, preferably metazoan, more preferably mammalian, most preferably human cells.

The target cell can be located in vitro, ex vivo or in vivo. One advantage of the invention is that it can be used with cells in situ in a living organism, but it can also be used with cells in culture. In some embodiments cells are treated ex vivo and are then introduced into a living organism (e.g., re-introduced into an organism from whom they were originally derived). The invention can also be used to edit target RNA sequences in cells from a transplant or within a so-called organoid, e.g., a liver tissue organoid. Organoids can be thought of as three-dimensional in vitro-derived tissues but are driven using specific conditions to generate individual, isolated tissues. In a therapeutic setting they are useful because they can be derived in vitro from a patient's cells, and the organoids can then be re-introduced to the patient as autologous material which is less likely to be rejected than a normal transplant.

Without wishing to be bound by theory, the RNA editing through hADAR2 is thought to take place on primary transcripts in the nucleus, during transcription or splicing, or in the cytoplasm, where e.g., mature mRNA, miRNA or ncRNA can be edited.

Generally spoken, RNA editing may be used to create RNA sequences with different properties. Such properties may be coding properties (creating proteins with different sequences or length, leading to altered protein properties or functions), or binding properties (causing inhibition or over-expression of the RNA itself or a target or binding partner; entire expression pathways may be altered by recoding miRNAs or their cognate sequences on target RNAs). Protein function or localization may be changed at will, by functional domains or recognition motifs, including but not limited to signal sequences, targeting or localization signals, recognition sites for proteolytic cleavage or co- or post-translational modification, catalytic sites of enzymes, binding sites for binding partners, signals for degradation or activation and so on. These and other forms of RNA and protein “engineering”, whether to prevent, delay or treat disease or for any other purpose, in medicine or biotechnology, as diagnostic, prophylactic, therapeutic, research tool or otherwise, are encompassed by the present invention.

The amount of triterpene saponin as well as the amount of EON to be administered, the dosage and the dosing regimen can vary from cell type to cell type, the disease to be treated, the target population, the mode of administration (e.g., systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change, or a change in (the level of, or activity of) a specified biomarker. It is possible that higher doses of EONs could compete for binding to an ADAR within a cell, thereby depleting the amount of the entity, which is free to take part in RNA editing, but routine dosing trials will reveal any such effects for a given EON and a given target. The same holds true for the amount of triterpene saponin that is administered, which may differ based on the amount of EON, the tissue to be treated, or the (human) subject that is in need of treatment, for instance because of weight, age, gender, etc.

One suitable trial technique involves delivering the EON to cell lines, or a test organism and then taking biopsy samples at various time points thereafter. The sequence of the target RNA can be assessed in the biopsy sample and the proportion of cells having the modification can easily be followed. After this trial has been performed once then the knowledge can be retained, and future delivery can be performed without needing to take biopsy samples. A method of the invention can thus include a step of identifying the presence of the desired change in the cell's target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively, the change may be assessed on the function of the protein, before and after treatment, or any other potential marker, which measurements are preferably performed in vitro on samples obtained from the treated subject.

After RNA editing has occurred in a cell, the modified RNA can become diluted over time, for example due to cell division, limited half-life of the edited RNAs, etc. Thus, in practical therapeutic terms a method of the invention may involve repeated delivery of an EON until enough target RNAs have been modified to provide a tangible benefit to the patient and/or to maintain the benefits over time.

The composition and kit-of-parts of the invention are particularly suitable for therapeutic use, and so the invention also relates to a pharmaceutical composition comprising an EON and a pharmaceutically acceptable carrier, whereas the triterpene glycoside may be combined in that same pharmaceutical composition or may be kept separate in a kit-of-parts when the triterpene glycoside and the EON are administered at different points in time. Hence, the kit-of-parts may comprise a triterpene glycoside in a suitable administrable composition, and separately an EON in a suitable administrable composition, both of which may comprise their respective suitable solvent, carrier, diluent, etc. When the EON and the triterpene glycoside are combined in a single pharmaceutical composition, when the EON and triterpene glycoside are administered at the same time, the specific solvent, carrier, or diluent may be the same or different. In some embodiments of the invention the pharmaceutically acceptable carrier can simply be a saline solution. This can usefully be isotonic or hypotonic, particularly for pulmonary delivery. The invention also provides a delivery device (e.g., syringe, inhaler, nebuliser) which includes a pharmaceutical composition of the invention. The EON is suitably administrated in aqueous solution, e.g., saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be intranasally, orally, by injection or infusion, intravenously, subcutaneously, intradermally, intramuscularly, intra-tracheally, intra-peritoneally, intrarectally, intrathecally, intra-cisterna magna, parenterally, and the like. Administration may be in solid form, in the form of a powder, a pill, a gel, a solution, a slow-release formulation, or in any other form compatible with pharmaceutical use in humans.

The invention provides an EON and a triterpene glycoside, preferably AG1856, for use in the treatment of any disorder that can be treated by deaminating a specific adenosine in a specific target transcript molecule. This treatment can be achieved through making a change in a target RNA sequence in a mammalian, such as a human liver cell, but is not necessarily limited thereto. Similarly, the invention provides the use of an EON+a triterpene glycoside, preferably AG1856, in the manufacture of a medicament for making a change in a target RNA sequence in a mammalian, preferably a human liver cell, as described herein, and thereby treating, preventing, or ameliorating disease.

The invention also relates to a method for the deamination of at least one specific target adenosine present in a target RNA molecule (mRNA or pre-mRNA) in a cell, the method comprising the steps of: providing the cell with a triterpene glycoside, preferably AG1856, and providing the cell with an EON; allowing uptake by the cell of the EON; allowing the endosomal release of the EON; allowing annealing of the EON to the target RNA molecule; allowing a mammalian ADAR enzyme comprising a natural dsRNA binding domain as found in the wild type enzyme to deaminate the target adenosine in the target RNA molecule to an inosine; and optionally identifying the presence of the inosine in the RNA sequence.

In a preferred aspect, depending on the ultimate effect of A to I conversion, the identification step comprises the following steps: sequencing the target RNA; assessing the presence or absence of an A to G conversion in target RNA derived cDNA; assessing the presence or absence of a functional protein; assessing whether splicing of the pre-mRNA was altered by the deamination; or using a functional read-out. A very suitable manner to identify the presence of an inosine after deamination of the target adenosine is of course dPCR or even sequencing, using methods that are well-known to the person skilled in the art.

In one embodiment, a method according to the invention comprises the steps of administering to the subject a triterpene glycoside and an EON, allowing the formation of a double stranded nucleic acid complex of the EON with its specific complementary target nucleic acid molecule in a cell in the subject; allowing the engagement of an endogenous present adenosine deaminating enzyme, such as ADAR1 and/or ADAR2; and allowing the enzyme to deaminate the target adenosine in the target nucleic target molecule to an inosine, thereby alleviating, preventing or ameliorating disease.

The composition according to the invention, or the kit-of-parts according to the invention can be applied in any type of disease that can beneficially be treated by ADAR-mediated deamination of a target adenosine present in a target transcript molecule that is involved in the disease. Non-limiting examples are:
Hypercholesterolemia, wherein the target adenosine is in the codon encoding position 152 of the human PCSK9 proprotein (see PCT/EP2023/053503);
Hurler syndrome, wherein the target adenosine is the c. 1205G>A mutation in the human IDUA gene (see e.g., WO2021/209010);
HFE hemochromatosis, wherein the target adenosine is a c.845G>A mutation in the human HFE gene (see PCT/EP2023/082797, unpublished); and
Cardiovascular disease (CVD), wherein the target adenosine is at position c. 1055A in the human B4GALT1 transcript (see PCT/EP2023/084865, unpublished).

Other examples of target transcripts and related diseases are USH2A (Usher syndrome), APP, NTCP, CMT1A, LRRK2, ASS1, GJB2, MECP2, OTOF (autosomal recessive non-syndromic hearing loss), XLRS, argininosuccinate lyase deficiency, ABCA4 (Stargardt disease), and SERPINA1 (A1AT deficiency).

### Chemical Modifications

All chemical modifications listed below that may be used in the EON of the present invention may also be used for a sense strand that is complementary to the EON, when the EON and the complementary strand form a so-called heteroduplex RNA editing oligonucleotide (HEON) complex, as described in GB 2215614.5 (not published), except that the opposite sense strand does not have an orphan nucleotide. It should be noted that the ‘orphan nucleotide’ is a definition of the nucleotide that is (when the EON is bound to the target sequence in the cell) directly opposite the target adenosine in the target RNA molecule. The orphan nucleotide does not necessarily mismatch with the target adenosine, for instance when the nucleotide opposite the target adenosine is a uridine. In nature, when ADAR is active, the orphan nucleotide is generally a cytidine, when it opposes the target adenosine. Hence, the definition is only used for its position, not because of any chemical modifications or characteristics of that nucleotide. The orphan nucleotide is only present in the guide oligonucleotide, the EON, not in the sense strand when the EON is bound to a complementary or partially complementary sense strand. Hence, the modification related to the orphan nucleotide relate only to the EON of the present invention, but all other modifications relate to the EON of the present invention and any (protecting) sense oligonucleotide that may be used together with the EON in a pharmaceutical product. This includes the use of hydrophobic moieties (such as tocopherol and cholesterol) and cell-specific ligands (such as GalNAc moieties), that have also been described herein, and in detail in GB 2215614.5 (not published), which may either be bound as single or multiple copies to the EON or its opposite strand, or both, at various locations.

The internucleoside linkages in the oligonucleotides of the present invention may comprise one or more naturally occurring internucleoside linkages and/or modified internucleoside linkages. Without limitations, at least one, at least two, or at least three internucleoside linkages from a 5′ and/or 3′ end of the EON are preferably modified internucleoside linkages. A preferred modified internucleoside linkage is a PS linkage. In one embodiment, all internucleoside linkages of the EON are modified internucleoside linkages. In one embodiment, the EON comprises a phosphoryl guanidine linkage, such as a PNdmi linkage, linking the most terminal nucleoside at the 5′ and/or 3′ end, and the one before last nucleoside at each of these ends, respectively. A PNdmi linkage as preferably used in the EONs of the present invention has the structure of formula (III):

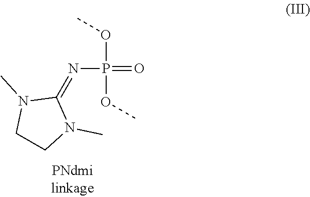

A common limiting factor in oligonucleotide-based therapies are the oligonucleotide's ability to be taken up by the cell (when delivered per se, or ‘naked’ without applying a delivery vehicle), its biodistribution and its resistance to nuclease-mediated breakdown. The skilled person is aware, and it has been described in detail in the art, that a variety of chemical modifications can assist in overcoming such limitations. Examples of such now commonly used chemical modifications are the 2′-O-methyl (often abbreviated to 2′-OMe or 2′-O-Me), 2′-F and 2′-O-methoxyethyl (often also referred to as 2′-methoxyethoxy, or 2′-MOE) modifications of the sugar and the use of PS linkages between nucleosides. WO2020/201406 discloses the use of MP linkage modifications at certain positions surrounding the orphan nucleotide in the first nucleic acid strand. The ribose 2′ groups in all nucleotides of the EON, except for the ribose sugar moiety of the orphan nucleotide that has certain limitations in respect of compatibility with RNA editing, can be independently selected from 2′-H (i.e., DNA), 2′-OH (i.e., RNA), 2′-OMe, 2′-MOE, 2′-F, or 2′-4′-linked (for instance a locked nucleic acid (LNA)), or other ribosyl 1′-substitutions, 2′ substitutions, 3′ substitutions, 4′ substitutions or 5′ substitutions. The orphan nucleotide in the EON that comprises no other chemical modifications to the ribose sugar, the base, or the linkage preferably does not carry a 2′-OMe or 2′-MOE substitution but may carry a 2′-F, a 2′,2′-disubstituted sugar (such as a 2′,2′-difluoro (diF), or 2′-fluoro-2′-methyl) or 2′-ara-F (FANA) substitution or may be DNA. GB 2214347.3 (not published) describes the modification of the 2′ position of the ribose sugar moiety of the orphan nucleotide by a 2′,2′-disubstituted substitution such as diF, which is also applicable to the invention described here. The 2′-4′ linkage can be selected from many linkers known in the art, such as a methylene linker, amide linker, or constrained ethyl linker (cEt).

The invention relates to an EON for use in the deamination of a target nucleotide (preferably adenosine) in a target RNA, wherein the EON is complementary to a stretch of nucleotides in the target RNA that includes the target adenosine, wherein the nucleotide in the first nucleic acid strand that is directly opposite the target nucleotide is the orphan nucleotide, and when the target nucleotide is an adenosine the orphan nucleotide comprises preferably a base or modified base or base analogue with a NH moiety at the position similar to the ring nitrogen (e.g., Benner's base Z). The nucleotide numbering in the EON is such that the orphan nucleotide is number 0 and the nucleotide 5′ from the orphan nucleotide is number +1. Counting is further positively (+) incremented towards the 5′ end and negatively (−) incremented towards the 3′ end, wherein the first nucleotide 3′ from the orphan nucleotide is number −1. The internucleoside linkage numbering in the EON is such that linkage number 0 is the linkage 5′ from the orphan nucleotide, and the linkage positions in the oligonucleotide are positively (+) incremented towards the 5′ end and negatively (−) incremented towards the 3′ end.

Preferably, the EON comprises one or more (chirally pure or chirally mixed) PS linkages. In one embodiment, the PS linkages connect the terminal 3, 4, 5, 6, 7, or 8 nucleotides on each end of the first nucleic acid strand. In one embodiment, the EON comprises one or more phosphoramidate (PN) linkages. In one embodiment, a PN linkage connects the terminal two nucleotides on each end of the EON.

A nucleoside in the EON may be a natural nucleoside (deoxyribonucleoside or ribonucleoside) or a non-natural nucleoside. It is noted that for RNA editing, in which double-stranded RNA is generally the substrate for enzymes with deamination activity (such as ADARs), ribonucleosides are considered ‘natural’, while deoxyribonucleosides may then be, for the sake of argument, considered as non-natural, or modified, simply because DNA is not present in the RNA-RNA double stranded substrate configurations. The skilled person appreciates that when the nucleotide has a natural ribose moiety, it may still be non-naturally modified in the base and/or the linkage.

In addition to the specific preferred chemical modifications at certain positions in compounds of the invention, compounds of the invention may comprise one or more (additional) modifications to the nucleobase, scaffold and/or backbone linkage, which may or may not be present in the same monomer, for instance at the 3′ and/or 5′ position. A scaffold modification indicates the presence of a modified version of the ribosyl moiety as naturally occurring in RNA (i.e., the pentose moiety), such as bicyclic sugars, tetrahydropyrans, hexoses, morpholinos, 2′-modified sugars, 4′-modified sugars, 5′-modified sugars and 4′-substituted sugars. Examples of suitable modifications include, but are not limited to 2′-O-modified RNA monomers, such as 2′-O-alkyl or 2′-O-(substituted) alkyl such as 2′-OMe, 2′-O-(2-cyanoethyl), 2′-MOE, 2′-O-(2-thiomethyl)ethyl, 2′-O-butyryl, 2′-O-propargyl, 2′-O-allyl, 2′-O-(2-aminopropyl), 2′-O-(2-(dimethylamino) propyl), 2′-O-(2-amino)ethyl, 2′-O-(2-(dimethylamino)ethyl); 2′-deoxy (DNA); 2′-O-(haloalkyl) methyl such as 2′-O-(2-chloroethoxy) methyl (MCEM), 2′-O-(2,2-dichloroethoxy) methyl (DCEM); 2′-O-alkoxycarbonyl such as 2′-O-[2-(methoxycarbonyl)ethyl] (MOCE), 2′-O-[2-N-methylcarbamoyl)ethyl] (MCE), 2′-O-[2-(N,N-dimethylcarbamoyl)ethyl] (DCME); 2′-halo e.g. 2′-F, FANA; 2′-O-[2-(methylamino)-2-oxoethyl] (NMA); a bicyclic or bridged nucleic acid (BNA) scaffold modification such as a conformationally restricted nucleotide (CRN) monomer, a locked nucleic acid (LNA) monomer, a xylo-LNA monomer, an a-LNA monomer, an α-I-LNA monomer, a β-d-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-O,4′-C) constrained ethyl (cEt) BNA monomer, a (2′-O,4′-C) constrained methoxyethyl (cMOE) BNA monomer, a 2′,4′-BNA^{NC} (NH) monomer, a 2′,4′-BNA^{NC} (NMe) monomer, a 2′,4′-BNA^{NC} (NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-I-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above. All these modifications are known to the person skilled in the art.

The base sequence of the EON herein is complementary to part of the base sequence of a target transcription product that includes at least a target adenosine that is to be deaminated to an inosine, and therefore can anneal (or hybridize) to the target transcription product. The complementarity of a base sequence can be determined by using a BLAST program or the like. Those skilled in the art can easily determine the conditions (temperature, salt concentration, and the like) under which two strands can be hybridized, taking into consideration the complementarity between the strands.

The EON according to the present invention, in contrast to what has been described for gapmers and their relation towards RNase breakdown and the use of such gapmers in double-stranded complexes (see for instance EP 3954395 A1), does not comprise a stretch of DNA nucleotides which would make a target sequence (or a sense nucleic acid strand) a target for RNase-mediated breakdown. In one embodiment, the EON does not comprise four or more consecutive DNA nucleotides anywhere within its sequence. In an embodiment, the EON is composed of as much (chemically) modified nucleotides as possible to enhance the resistance towards RNase-mediated breakdown, while at the same time being as efficient as possible in producing an RNA editing effect. This means that the orphan nucleotide and several other nucleotides within the EON may be DNA, but also that there is no stretch of four or more consecutive DNA nucleotides within the EON. Hence, the EON according to the present invention is not a gapmer. A gapmer is in principle a single-stranded nucleic acid consisting of a central region (DNA gap region with at least four consecutive deoxyribonucleotides) and wing regions positioned directly at the 5′ end (5′ wing region) and the 3′ end (3′ wing region) thereof. In contrast, the EON according to the invention may be any oligonucleotide that produces an RNA editing effect in which a target adenosine in a target RNA molecule is deaminated to an inosine, and accordingly is resistant to RNase-mediated breakdown as much as possible to yield this effect.

In one embodiment, the EON, or the sense strand to which it may be annealed before entering a target cell, is bound to a hydrophobic moiety, such as palmityl or an analog thereof, cholesterol or analog thereof, or tocopherol or analog thereof. It is preferably bound to the 5′ terminus. In case a hydrophobic moiety is bound to the 5′ terminus as well as to the 3′ terminus, such hydrophobic moieties may be the same or different. The hydrophobic moiety bound to the oligonucleotide may be bound directly, or indirectly mediated by another substance. When the hydrophobic moiety is bound directly, it is sufficient if the moiety is bound via a covalent bond, an ionic bond, a hydrogen bond, or the like. When the hydrophobic moiety is bound indirectly, it may be bound via a linking group (a linker). The linker may be a cleavable or an uncleavable linker. A cleavable linker refers to a linker that can be cleaved under physiological conditions, for example, in a cell or an animal body (e.g., a human body). A cleavable linker is selectively cleaved by an endogenous enzyme such as a nuclease, or by physiological circumstances specific to parts of the body or cell, such as pH or reducing environment (such as glutathione concentrations). Examples of a cleavable linker comprise, but is not limited to, an amide, an ester, one or both esters of a phosphodiester, a phosphoester, a carbamate, and a disulfide bond, as well as a natural DNA linker. Cleavable linkers also include self-immolative linkers. An uncleavable linker refers to a linker that is not cleaved under physiological conditions, or very slowly compared to a cleavable linker, for example, in a PS linkage, modified or unmodified deoxyribonucleosides linked by a PS linkage, a spacer connected through a PS bond and a linker consisting of modified or unmodified ribonucleosides. There is no restriction on the chain length, when a linker is a nucleic acid such as DNA, or an oligonucleotide. However, it may be usually from 2 to 20 bases in length, from 3 to 10 bases in length, or from 4 to 6 bases in length. There is no restriction on the length or composition of a spacer that connects the ligand and the oligonucleotide, and may include for example ethylene glycol, TEG, HEG, alkyl chains, propyl, 6-aminohexyl, or dodecyl.

In an embodiment, the EON for RNA editing is administered separately from the triterpene glycoside, which is preferably AG1856. This may be in the order of first administering the triterpene glycoside, followed by administration of the EON, or first wherein the EON is administered, followed by the triterpene glycoside. It may also be administered at the same time, wherein it is preferred that the EON and the triterpene glycoside are in the same composition for administration. In another embodiment, the triterpene glycoside is covalently, or non-covalently bound to the EON.

The invention also relates to a pharmaceutical composition comprising the EON according to the invention, and further comprising a pharmaceutically acceptable carrier and/or other additive and may be dissolved in a pharmaceutically acceptable organic solvent, or the like. Dosage forms in which the EON or the pharmaceutical composition are administered may depend on the disorder to be treated and the tissue that needs to be targeted and can be selected according to common procedures in the art. The pharmaceutical compositions may be administered by a single-dose administration or by multiple dose administration. It may be administered daily or at appropriate time intervals, which may be determined using common general knowledge in the field and may be adjusted based on the disorder and the efficacy of the active ingredient. Consequently, the order in which the triterpene glycoside is administered may also be varied.

In one embodiment, the EON comprises at least one nucleotide with a sugar moiety that comprises a 2′-OMe modification. In one embodiment, the EON comprises at least one nucleotide with a sugar moiety that comprises a 2′-MOE modification. In one embodiment, the EON comprises at least one nucleotide with a sugar moiety that comprises a 2′-F modification. In one embodiment, the orphan nucleotide carries a 2′-H in the sugar moiety and is therefore referred to as a DNA nucleotide, even though additional modifications may exist in its base and/or linkage to its neighbouring nucleosides. In one embodiment, the orphan nucleotide carries a 2′-F in the sugar moiety. In one embodiment, the orphan nucleotide carries a diF substitution in the sugar moiety. In one embodiment, the orphan nucleotide carries a 2′-F and a 2′-C-methyl in the sugar moiety. In one embodiment, the orphan nucleotide comprises a 2′-F in the arabinose configuration (FANA) in the sugar moiety. In one embodiment, the EON is an antisense oligonucleotide that can form a double stranded nucleic acid complex with a target RNA molecule, wherein the double stranded nucleic acid complex can recruit an adenosine deaminating enzyme for deamination of a target adenosine in the target RNA molecule, wherein the nucleotide in the EON that is opposite the target adenosine is the orphan nucleotide, and wherein the orphan nucleotide is according to formula (IV):

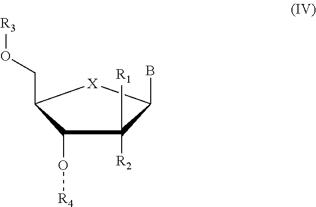

wherein: X is O, NH, OCH₂, CH₂, Se, or S; B is a nitrogenous base selected from the group consisting of: cytosine, uracil, isouracil, N3-glycosylated uracil, pseudoisocytosine, 8-oxo-adenine, and 6-amino-5-nitro-2 (1H)-pyridone; R₁ and R₂ are both selected, independently, from H, OH, F or CH₃; R₃ is the part of the EON that is 5′ of the orphan nucleotide, consisting of 7 to 30 nucleotides; and R₄ is the part of the EON that is 3′ of the orphan nucleotide, consisting of 4 to 25 nucleotides. The nucleotide 3′ and/or 5′ from the orphan nucleotide may be DNA, more preferably the nucleotide at the 3′ (position −1).

In one embodiment, the first nucleic acid strand comprises at least one MP internucleoside linkage according to formula (V):

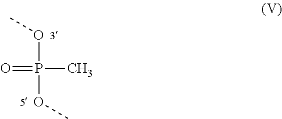

A preferred position for an MP linkage in an EON according to the invention is linkage position −1, thereby connecting the nucleoside at position −1 with the nucleoside at position −2, although other positions for MP linkages are not explicitly excluded.

In one embodiment, the EON comprises at least one nucleotide with a sugar moiety that comprises a 2′-fluoro (2′-F) modification. A preferred position for the nucleotide that carries a 2′-F modification is position −3 in EON, which may be present together with an identical 2′ modification in the orphan nucleotide as discussed above.

In one embodiment, the EON comprises at least one phosphonoacetate or phosphonoacetamide internucleoside linkage.

In one embodiment, the EON comprises at least one nucleotide comprising a locked nucleic acid (LNA) ribose modification, or an unlocked nucleic acid (UNA) ribose modification. In an embodiment, the EON comprises at least one nucleotide comprising a threose nucleic acid (TNA) ribose modification.

The skilled person knows that an oligonucleotide, such as an EON as outlined herein, generally consists of repeating monomers. Such a monomer is most often a nucleotide or a chemically modified nucleotide. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide.

A modification in the pentose sugar is therefore often referred to as a ‘scaffold modification’. The original pentose sugar may be replaced in its entirety by another moiety that similarly connects the base and the phosphate. It is therefore understood that while a pentose sugar is often a scaffold, a scaffold is not necessarily a pentose sugar. Examples of scaffold modifications that may be applied in the monomers of the EON of the present invention are disclosed in WO2020/154342, WO2020/154343, and WO2020/154344.

In one embodiment, the EON of the present invention may comprise one or more nucleotides carrying a 2′-MOE ribose modification. Also, in one embodiment, the EON comprises one or more nucleotides not carrying a 2′-MOE ribose modification, and wherein the 2′-MOE ribose modifications are at positions that do not prevent the enzyme with adenosine deaminase activity from deaminating the target adenosine. In another embodiment, the EON comprises 2′-OMe ribose modifications at the positions that do not comprise a 2′-MOE ribose modification, and/or wherein the oligonucleotide comprises deoxynucleotides at positions that do not comprise a 2′-MOE ribose modification. In one embodiment the EON comprises one or more nucleotides comprising a 2′ position comprising a 2′-MOE, 2′-OMe, 2′-OH, 2′-deoxy, TNA, 2′-fluoro (2′-F), a 2′,2′-disubstituted modification (such as a 2′,2′-difluoro (diF) modification, a 2′-fluoro-2′-C-methyl modification, or others such as those indicated in e.g., Grosse et al, (ACS Med Chem Lett 2022 DOI: 10.1021/acsmedchemlett.2c00372) including 2′-spirocyclic ones) or a 2′-4′-linkage (i.e., a bridged nucleic acid such as a locked nucleic acid (LNA or examples mentioned in e.g. WO2018/007475)). In another embodiment, other nucleic acid monomers that are applied are arabinonucleic acids and 2′-deoxy-2′-fluoroarabinonucleic acid (FANA), for instance for improved affinity purposes. The 2′-4′ linkage can be selected from linkers known in the art, such as a methylene linker or constrained ethyl linker. A wide variety of 2′ modifications are known in the art. Further examples are disclosed in further detail in WO2016/097212, WO2017/220751, WO2018/041973, WO2018/134301, WO2019/219581, WO2019/158475, and WO2022/099159 for instance. In all cases, the modifications should be compatible with editing such that the EON fulfils its role as an editing producing oligonucleotide that can form a double stranded complex with the target RNA and recruit a deaminating enzyme, that can subsequently deaminate the target adenosine. Where a monomer comprises an unlocked nucleic acid (UNA) ribose modification, that monomer can have a 2′ position comprising the same modifications discussed above, such as a 2′-MOE, a 2′-OMe, a 2′-OH, a 2′-deoxy, a 2′-F, a 2′,2′-diF, a 2′-fluoro-2′-C-methyl, an arabinonucleic acid, a FANA, or a 2′-4′-linkage (i.e., a bridged nucleic acids such as a locked nucleic acid (LNA)).

A base, sometimes called a nucleobase, is generally adenine, cytosine, guanine, thymine or uracil, or a derivative thereof. A base, sometimes called a nucleobase, is defined as a moiety that can bond to another nucleobase through H-bonds, polarized bonds (such as through CF moieties) or aromatic electronic interactions. Cytosine, thymine, and uracil are pyrimidine bases, and are generally linked to the scaffold through their 1-nitrogen. Adenine and guanine are purine bases and are generally linked to the scaffold through their 9-nitrogen. The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy) ribosyl sugar.

The nucleobases in an EON of the present invention can be adenine, cytosine, guanine, thymine, or uracil or any other moiety able to interact with another nucleobase through H-bonds, polarized bonds (such as CF) or aromatic electronic interactions. The nucleobases at any position in the nucleic acid strand can be a modified form of adenine, cytosine, guanine, or uracil, such as hypoxanthine (the nucleobase in inosine), pseudouracil, pseudocytosine, isouracil, N3-glycosylated uracil, isocytosine, 1-methylpseudouracil, orotic acid, agmatidine, lysidine, 2-thiouracil, 2-thiothymine, 5-substituted pyrimidine (e.g., 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine), 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, 8-oxo-adenine, 3-deazapurine (such as a 3-deaza-adenosine), pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp and its derivatives, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose).

In an embodiment, the nucleotide analog is an analog of a nucleic acid nucleotide. In an embodiment, the nucleotide analog is an analog of adenosine, guanosine, cytidine, thymidine, uridine, deoxyadenosine, deoxyguanosine, deoxycytidine, deoxythymidine or deoxyuridine. In an embodiment, the nucleotide analog is not guanosine or deoxyguanosine. In an embodiment, the nucleotide analog is not a nucleic acid nucleotide. In an embodiment, the nucleotide analog is not adenosine, guanosine, cytidine, thymidine, uridine, deoxyadenosine, deoxyguanosine, deoxycytidine, deoxythymidine, or deoxyuridine.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked scaffolds of an oligonucleotide is often called the ‘backbone’ of the oligonucleotide. Because phosphodiester bonds connect neighboring monomers together, they are often referred to as ‘backbone linkages’. It is understood that when a phosphate group is modified so that it is instead an analogous moiety such as a PS, such a moiety is still referred to as the backbone linkage of the monomer. This is referred to as a ‘backbone linkage modification’. In general terms, the backbone of an oligonucleotide comprises alternating scaffolds and backbone linkages.

EONs according to the invention can comprise linkage modifications. A linkage modification can be, but is not limited to, a modified version of the phosphodiester present in RNA, such as PS, chirally pure PS, (R)-PS, (S)-PS, methyl phosphonate (MP), chirally pure methyl phosphonate, (R)-methyl phosphonate, (S)-methyl phosphonate, phosphoryl guanidine (such as PNdmi), chirally pure phosphoryl guanidine, (R)-phosphoryl guanidine, (S)-phosphoryl guanidine, phosphorodithioate (PS2), phosphonacetate (PACE), phosphonoacetamide (PACA), thiophosphonoacetate, thiophosphonoacetamide, methyl phosphorohioate, methyl thiophosphonate, PS prodrug, alkylated PS, H-phosphonate, ethyl phosphate, ethyl PS, boranophosphate, borano PS, methyl boranophosphate, methyl borano PS, methyl boranophosphonate, methyl boranophosphothioate, phosphate, phosphotriester, aminoalkylphosphotriester, and their derivatives. Another modification includes phosphoramidite, phosphoramidate, N3′→P5′ phosphoramidate, phosphorodiamidate, phosphorothiodiamidate, sulfamate, diethylenesulfoxide, amide, sulfonate, siloxane, sulfide, sulfone, formacetyl, alkenyl, methylenehydrazino, sulfonamide, triazole, oxalyl, carbamate, methyleneimino (MMI), and thioacetamide nucleic acid (TANA); and their derivatives. Various salts, mixed salts and free acid forms are also included, as well as 3′→3′ and 2′→5′ linkages.

In one embodiment, an EON comprises a substitution of one of the non-bridging oxygens in the phosphodiester linkage. This modification slightly destabilizes base-pairing but adds significant resistance to nuclease degradation. A preferred nucleotide analogue or equivalent comprises PS, phosphonoacetate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate. Particularly preferred are internucleoside linkages that are modified to contain a PS. Many of these non-naturally occurring modifications of the linkage, such as PS are chiral, which means that there are Rp and Sp configurations, known to the person skilled in the art. In one embodiment, the chirality of the PS linkages is controlled, which means that each of the linkages is either in the Rp or in the Sp configuration, whichever is preferred. The choice of an Rp or Sp configuration at a specified linkage position may depend on the target sequence and the efficiency of binding and induction of providing RNA editing. However, if such is not specifically desired, a composition may comprise AONs as active compounds with both Rp and Sp configurations at a certain specified linkage position. Mixtures of such EONs are also feasible, wherein certain positions have preferably either one of the configurations, while for other positions such does not matter.

Again, in all cases, the modifications should be compatible with editing such that the EON fulfils its role as an editing producing oligonucleotide that can, when attached to its target sequence, recruit an adenosine deaminase enzyme because of the dsRNA nature that arises. In all aspects of the invention, the enzyme with adenosine deaminase activity is preferably ADAR1, ADAR2, or ADAT. In a highly preferred embodiment, the EON is an RNA editing oligonucleotide that targets a pre-mRNA or an mRNA, wherein the target nucleotide is an adenosine in the target RNA, wherein the adenosine is deaminated to an inosine, which is being read as a guanosine by the translation machinery. The invention also relates to a pharmaceutical composition comprising the EON as characterized herein, and a pharmaceutically acceptable carrier.

Other chemical modifications of the EON according to the invention include the substitution of one or more than one of any of the hydrogen atoms with deuterium or tritium, examples of which can be found in e.g., WO2014/022566 or WO2015/011694.

EONs of the present invention preferably do not include a 5′-terminal 06-benzylguanosine or a 5′-terminal amino modification and preferably are not covalently linked to a SNAP-tag domain (an engineered O6-alkylguanosine-DNA-alkyl transferase). EONs of the present invention preferably do not comprise a boxB RNA hairpin sequence. In one embodiment, an EON of the present invention comprises 0, 1, 2 or 3 wobble base pairs with the target sequence, and/or 0, 1, 2, 3, 4, 5, 6, 7, or 8 mismatching base pairs with the target RNA sequence. No mismatch exists when the orphan nucleotide is uridine. One alternative for uridine is positioning an iso-uridine opposite the target adenosine, which likely does not pair like G pairs with U. Preferably, the target adenosine in the target sequence forms a mismatch base pair with the nucleoside in the EON that is directly opposite the target adenosine.

It should be noted that when an EON is delivered through a vector, for instance an AAV vector, chemical modifications are not present in the EON that acts on the target RNA molecule. According to the present invention, it is preferred to use ‘naked’ EONs that have chemical modifications as outlined herein. EONs that are circular, or have hairpin structures (recruiting portions, e.g., as disclosed in WO2016/097212, WO2017/050306, WO2020/001793, WO2017/010556, WO2020/246560, and WO2022/078995) are also encompassed by the present invention because these can also be applied to edit adenosines in the target RNA molecule.

An EON according to the present invention can utilise endogenous cellular pathways and naturally available ADAR enzymes to specifically edit a target adenosine in the target RNA sequence. An EON of the invention is capable of recruiting ADAR and complex with it and then facilitates the deamination of a (single) specific target adenosine nucleotide in a target RNA sequence. Ideally, only one adenosine is deaminated. An EON of the invention, when complexed to ADAR, preferably brings about the deamination of a single target adenosine.

Analysis of natural targets of ADAR enzymes has indicated that these generally include mismatches between the two strands that form the RNA helix edited by ADAR1 or 2. It has been suggested that these mismatches enhance the specificity of the editing reaction (Stefl et al. 2006. Structure 14 (2): 345-355; Tian et al. 2011. Nucleic Acids Res 39 (13): 5669-5681). Characterization of optimal patterns of paired/mismatched nucleotides between the EONs and the target RNA also appears important to the development of efficient ADAR-based EON therapy.

As outlined above, an EON of the present invention makes use of specific nucleotide modifications at predefined spots to ensure stability as well as proper ADAR binding and activity. These changes may vary and may include modifications in the backbone of the EON, in the sugar moiety of the nucleotides as well as in the nucleobases or the phosphodiester linkages, as outlined in detail herein. They may also be variably distributed throughout the sequence of the EON. Specific modifications may be needed to support interactions of different amino acid residues within the RNA-binding domains of ADAR enzymes, as well as those in the deaminase domain. For example, PS linkages between nucleotides or 2′-OMe or 2′-MOE modifications may be tolerated in some parts of the EON, while in other parts they should be avoided so as not to disrupt crucial interactions of the enzyme with the phosphate and 2′—OH groups. Specific nucleotide modifications may also be necessary to enhance the editing activity on substrate RNAs where the target sequence is not optimal for ADAR editing. Previous work has established that certain sequence contexts are more amenable to editing. For example, a target sequence 5′-UAG-3′ (with the target A in the middle) contains the most preferred nearest-neighbor nucleotides for ADAR2, whereas a 5′-CAA-3′ target sequence is disfavored (Schneider et al. 2014. Nucleic Acids Res 42 (10): e87). The structural analysis of ADAR2 deaminase domain hints at the possibility of enhancing editing by careful selection of the nucleotides that are opposite to the target trinucleotide. For example, the 5′-CAA-3′ target sequence, paired to a 3′-GCU-5′ sequence on the opposing strand (with the A-C mismatch formed in the middle), is disfavored because the guanosine base sterically clashes with an amino acid side chain of ADAR2.

Mutagenesis studies of human ADAR2 revealed that a single mutation at residue 488 from glutamate to glutamine (E488Q), gave an increase in the rate constant of deamination by 60-fold when compared to the wild-type enzyme (Kuttan and Bass. Proc Natl Acad Sci USA 2012. 109 (48): 3295-3304). During the deamination reaction, ADAR flips the edited base out of its RNA duplex, and into the enzyme active site (Matthews et al. 2016). When ADAR2 edits adenosines in the preferred context (an A: C mismatch) the nucleotide opposite the target adenosine is often referred to as the ‘orphan cytidine’. The crystal structure of ADAR2 E488Q bound to double stranded RNA (dsRNA) revealed that the glutamine (Gln) side chain at position 488 can donate an H-bond to the N3 position of the orphan cytidine, which leads to the increased catalytic rate of ADAR2 E488Q. In the wild-type enzyme, wherein a glutamate (Glu) is present at position 488 instead of a glutamine (Gln) the amide group of the glutamine is absent and is instead a carboxylic acid. To obtain the same contact of the orphan cytidine with the E488Q mutant would then, for the wild-type situation, require protonation for this contact to occur. To make use of endogenously expressed ADAR2 to correct disease relevant mutations, it is essential to maximize the editing efficiency of the wild type ADAR2 enzyme present in the cell. WO2020/252376 discloses the use of EONs with modified RNA bases, especially at the position of the orphan cytidine to mimic the hydrogen-bonding pattern observed by the E488Q ADAR2 mutant. By replacing the nucleotide opposite the target adenosine in the EON with cytidine analogs that serve as H-bond donors at N3, it was envisioned that it would be possible to stabilize the same contact that is believed to provide the increase in catalytic rate for the mutant enzyme. Two cytidine analogs were of particular interest: pseudoisocytidine (also referred to as ‘piC’; Lu et al. J Org Chem 2009. 74 (21): 8021-8030; Burchenal et al. (1976) Cancer Res 36:1520-1523) and Benner's base Z (also referred to as ‘dZ’; Yang et al. Nucl Acid Res 2006. 34 (21): 6095-6101) that were initially selected because they offer hydrogen-bond donation at N3 with minimal perturbation to the shape of the nucleobase. Benner's base is also chemically referred to as a 6-amino-5-nitro-3-yl-2 (1H)-pyridone nucleobase. The presence of the cytidine analog in the AON may exist in addition to modifications to the ribose 2′ group. The ribose 2′ groups in the AON can be independently selected from 2′-H (i.e., DNA), 2′-OH (i.e., RNA), 2′-OMe, 2′-MOE, 2′-F, or 2′-4′-linked (i.e., a bridged nucleic acid such as a locked nucleic acid (LNA)), or other 2′ substitutions. The 2′-4′ linkage can be selected from linkers known in the art, such as a methylene linker or constrained ethyl linker.

In one embodiment, an EON comprises one or more sugar moieties that are mono- or di-substituted at the 2′, 3′ and/or 5′ position such as: —OH; —H; —F; substituted or unsubstituted, linear or branched lower (C₁-C₁₀) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy; -dimethylamino oxyethoxy; and -dimethylaminoethoxyethoxy.

In one embodiment, a nucleotide analogue or equivalent within the EON comprises one or more base modifications or substitutions. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art. Purine nucleobases and/or pyrimidine nucleobases may be modified to alter their properties, for example by amination or deamination of the heterocyclic rings. The exact chemistries and formats may vary from oligonucleotide construct to oligonucleotide construct and from application to application, and may be worked out in accordance with the wishes and preferences of those of skill in the art.

An EON according to the invention is normally longer than 10 nucleotides, preferably more than 11, 12, 13, 14, 15, 16, still more preferably more than 17 nucleotides. In one aspect the AON according to the invention is longer than 20 nucleotides. The oligonucleotide according to the invention is preferably shorter than 100 nucleotides, still more preferably shorter than 60 nucleotides, still more preferably shorter than 50 nucleotides. In a preferred aspect, the oligonucleotide according to the invention comprises 18 to 70 nucleotides, more preferably comprises 18 to 60 nucleotides, and even more preferably comprises 18 to 50 nucleotides. Hence, in a particularly preferred aspect, the oligonucleotide of the present invention comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. In one embodiment, the AON is 27, 28, 29, or 30 nucleotides in length.

In one aspect, at either end or both termini of an EON according to the present invention inverted deoxyT or dideoxyT nucleotides are incorporated.

### EXAMPLES

### Example 1. Editing of a Target Adenosine in a Human APP RNA Molecule Using Triterpene Saponin AG1856 In Vitro

Human retinal pigment epithelium cell line ARPE-19 carrying the wild-type APP gene was obtained from ATCC (Lot. #70013110). Briefly, 5.0×10⁴ ARPE-19 cells were seeded per 24 well plate 4 h before oligo treatment. Then, 1 μM EON hAPPex17-1dZ was added to the cells. This EON (RM3023) has the following sequence:

5′-c*a*a*g*g*u*gaugacgau*c*a*c*u*gucgCdZAugaca*a*c*a*c*c*g*c-3′ (SEQ ID NO:1) in which * represents a PS linkage, dZ represents a deoxynucleotide with a Benner's base, C and A represent cytidine and adenosine (DNA) respectively and lower font nucleotides represent 2′-OMe modified nucleotides. This EON targets adenosine at position 2286 in exon 17 within the human APP mRNA. Non-treated (NT) ARPE-19 cells were taken as negative control, while another control were cells treated with EON alone (Control). 72 h after EON treatment, 2, 4 or 8 μM AG1856 (purified as disclosed in WO2021/122998) in fresh medium was added to the cells. RNA was extracted from cells 120 h after oligo treatment using the Direct-zol RNA MircoPrep (Zymo Research, R2062) kit according to the manufacturer's instructions, and cDNA was prepared using the Maxima reverse transcriptase kit (Thermo Fisher) according to the manufacturer's instructions, with a combination of random hexamer and oligo-dT primers. The cDNA was diluted 10× and 1 μL of this dilution was used as template for digital droplet PCR (ddPCR) with a total input of 2.5 ng RNA. The ddPCR assay for absolute quantification of nucleic acid target sequences was performed using BioRad's QX-200 Droplet Digital PCR system. 1 μl of diluted cDNA obtained from the RT cDNA synthesis reaction was used in a total mixture of 21 μl of reaction mix, including the ddPCR Supermix for Probes no dUTP (Bio Rad), a Taqman SNP genotype assay with the following forward and reverse primers combined with the gene-specific probes:

Hexachlorofluorescein, non-fluorescent quencher
labelled wild-type probe
(SEQ ID NO: 2)
5′-/5HEX/TGTT+GTCAT+A+G+CGACAGT/3IABkFQ/-3′
Fluorescein, non-fluorescent quencher labelled
mutant probe
(SEQ ID NO: 3)
5′-/56-FAM/TGTTGTCAT+G+GCGACAGT/3IABkFQ/-3′

A total volume of 21 μl PCR mix including cDNA was filled in the middle row of a ddPCR cartridge (BioRad) using a multichannel pipette. The replicates were divided by two cartridges. The bottom rows were filled with 70 μl of droplet generation oil for probes (BioRad). After the rubber gasket replacement, droplets were generated in the QX200 droplet generator. 42 μl of oil emulsion from the top row of the cartridge was transferred to a 96-wells PCR plate. The PCR plate was sealed with a tin foil for 4 sec at 170° C. using the PX1 plate sealer, followed by the following PCR program: 1 cycle of enzyme activation for 10 min at 95° C., 40 cycles denaturation for 30 sec at 95° C. and annealing/extension for 1 min at 53.8° C., 1 cycle of enzyme deactivation for 10 min at 98° C., followed by a storage at 8° C. The APP forward sequence primer was 5′-CATTGGACTCATGGTGG-3′ (SEQ ID NO:5). The APP reverse primer was 5′-CAGCATCACCAAGGTG-3′ (SEQ ID NO:6). After PCR, the plate was read and analysed with the QX200 droplet reader.

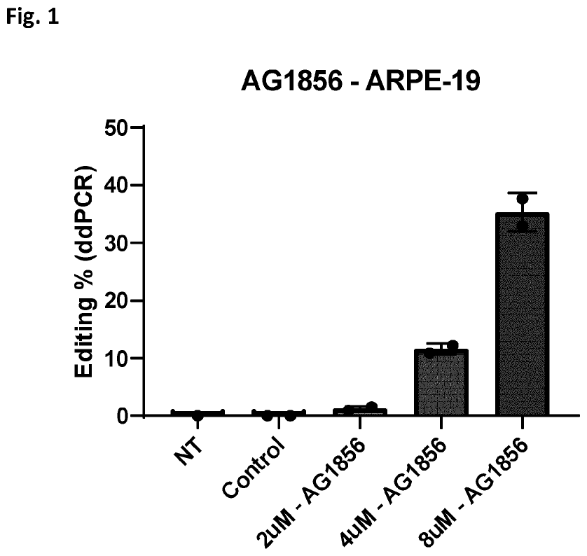

The effects on RNA editing are shown in FIG. 1 and clearly indicate a dose-dependent increase in RNA editing on the endogenous APP transcript in ARPE-19 cells using increasing amounts of AG1856, when delivered separately from the EON.

### Example 2. Editing of a Target Adenosine in a Mouse App RNA Molecule Using Triterpene Saponin AG1856 In Vivo

Female C57BL/6JRj mice were randomly assigned and all were around 10 weeks old at the first dose. Mice were group-housed (5 per cage) in standard open polysulfone type IIL cages on a regular diurnal lighting cycle (12h: 12 h light: dark) starting light at 07.00 am with ad libitum access to standard chow and water. A total of 12 female wildtype C57BL/6JRj mice were treated with a single subcutaneous (SC) injection at day 0 with AG1856 (3.6 mg/kg) in sterile PBS followed by an intravenous (IV) injection of EON targeting mApp adenosine at position 2344 in exon 17 (35 mg/kg) in sterile PBS 1 h later. The EON is named RM3835 and has the following sequence: 5′-a!u*c*a*c*G*g*U*u*G*c*dZ*A (MeP) u*G*a*C*a*A*c*G*c*c*g!c-3′ (SEQ ID NO:7) in which lower case letters represent 2′-OMe modified nucleotides, bold face A represents deoxyadenosine, underlined upper case letters represent 2′-F modified nucleotides, dZ represents a deoxynucleotide (DNA) carrying a Benner's base, underlined lower case letters represent 2′-MOE modified nucleotides, an asterisk represents PS linkages, (MeP) represents a methylphosphonate linkage, and “!” represents a PNdmi linkage.

Mice were sacrificed at day 14, and tissues were collected during necropsy. Snap-frozen tissue samples were thawed and disrupted in TRIzol reagent (Roche) using the MagNA lyzer (Roche). Samples were exposed to two MagNA lyzer runs of 30 sec each (6500 rpm), allowing for a 90 sec cool-off period in between runs. Samples were then incubated for 2 min at RT to allow complete dissociation of nucleoproteins. Subsequently, chloroform was added to the suspension preparing for phase separation. After centrifuging for 15 min at 12,000×g (4° C.), the aqueous phase that contained RNA was used for further processing. The ReliaPrep RNA Cell Miniprep System was used to isolate RNA following manufacturer's protocol. cDNA synthesis was performed using the Maxima Reverse Transcriptase kit (Thermo Scientific) following manufacturer's protocol. In short, to avoid cDNA synthesis interference due to secondary structures, 500 ng RNA was first incubated with dNTP mix (10 mM each), random hexamers and oligoDt at 70° C. for 5 min, then slowly cooled to 10° C. in 10° C. per 15 sec declines. Subsequently, reverse transcriptase buffer and enzyme was added, and samples were incubated at 25° C. for 10 min, 50° C. for 30 min and 80° C. for 5 min (to deactivate the enzyme). For dPCR analysis, cDNA samples (undiluted for pancreas, 40× diluted for other tissues) were incubated with a mix of primers and probes specific for wild type mApp exon 17 (HEX), mutant mApp exon 17 (FAM), total mApp exon 4-5 (Cy5) and mRps19. Primers and probe sequences were as follows:

| Target | Primer sequence (5′-3′) | Probe sequence (5′-3′) |
|---|---|---|
| mApp exon 17 | (Fw): CAACATCACCAGGGTGATGAC | Wild type: /5HEX/CGT T+GT CAT |
| | (SEQ ID NO: 8) | +A+G+C AAC CGT/3IABkFQ/ (SEQ |
| | (Rv): CATCATCGGACTCATGGTGG | ID NO: 10) |
| | (SEQ ID NO: 9) | Mutant: /56-FAM/CGT TGT CAT |
| | | +G+G+C AAC CG/3IABkFQ/ |
| | | (SEQ ID NO: 11) |
| mApp exon 4-5 | (Fw): GCGGATGGATGUTTGTGAGA | /5Cy5/CAC ACC +G+TC G+C+C |
| | (SEQ ID NO: 12) | AAA/3IAbRQSp/ |
| | (Rv): GCCACACTCTTAGAGCC | (SEQ ID NO: 14) |
| | (SEQ ID NO: 13) | |

+ = LNA

12 μl of each sample was loaded onto a QIAcuity nano plate 8.5K 96 wells and run on the QIAcuity (Qiagen) which includes sample partition, PCR, and imaging.

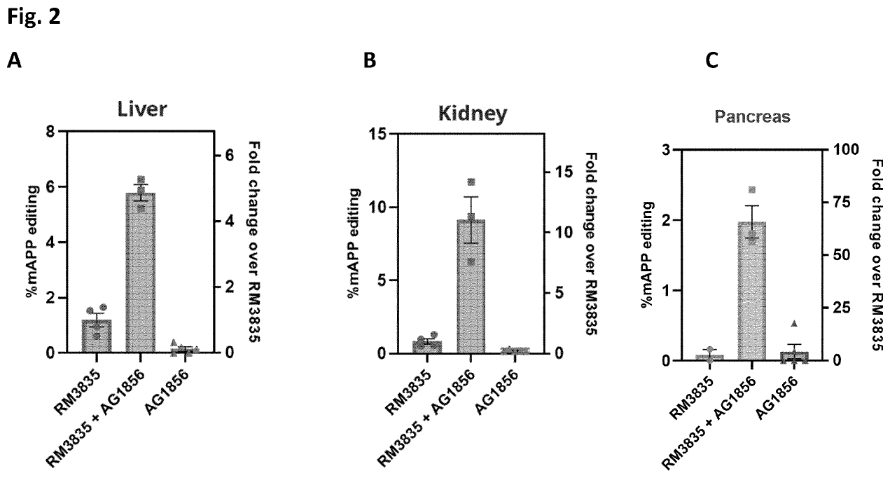

The results are shown in FIG. 2. Whereas no editing could be observed in the controls in which the triterpene saponin AG1856 was administered alone, some editing (0.1-1%) was observed in tissues from animals that were treated with RM3835 alone. This was increased to almost 2%, 6% and 9% in pancreas, liver, and kidney respectively when the triterpene saponin was pre-administered, demonstrating the strong effect of AG1856 on EON- and ADAR-mediated RNA editing efficiency in vivo.

### Example 3. Editing of Human B4GALT1 Transcripts in HepG2 Cells Using AG1856

A recently identified target in the fight against CVD is the enzyme B4GALT1, that is involved in the processing of biologically important biomolecules, including those that are involved in lipid metabolism and coagulation. B4GALT1 is ubiquitously expressed and plays a critical role in the processing of N-linked oligosaccharide moieties in glycoproteins, transferring the galactose from uridine diphosphate galactose (UDP-Gal) to specific glycoprotein substrates. B4GALT1 is therefore important for biological activity associated with correctly constructed oligosaccharides.

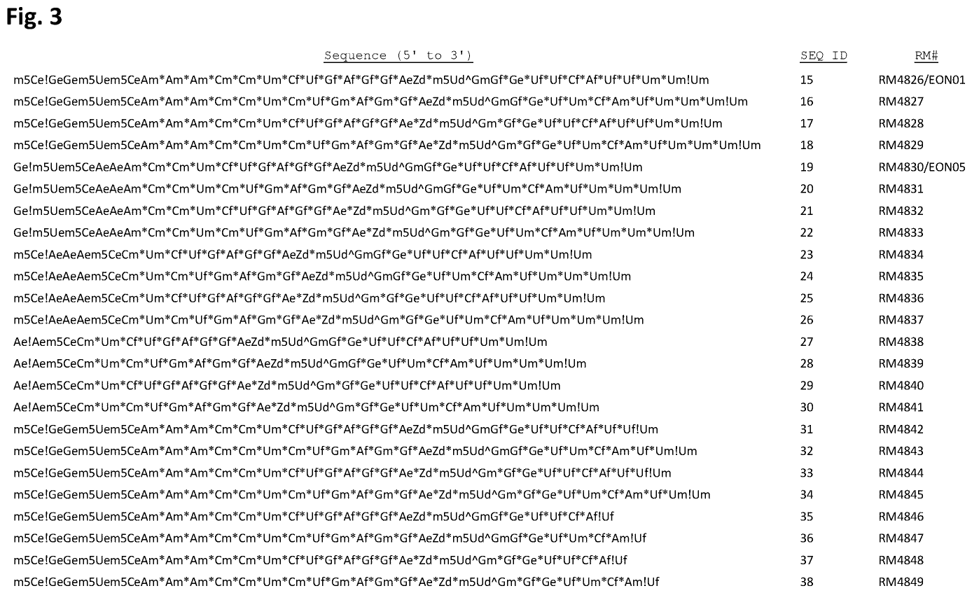

Editing of the endogenous human B4GALT1 transcript was investigated in human cells in the context of AG1856. For this, human HepG2 hepatocellular carcinoma cells were cultured in EMEM+10% FBS+1% P/S. Cells were kept at 37° C. in a 5% CO₂ atmosphere. In an initial experiment the EONs named RM4826 to RM4849 (see FIG. 3) were tested for editing efficiency. A total of 0.75×10⁵ HepG2 cells were seeded and treated with 1 or 5 μM EON, and 1 μM AG1856 was added per well. This mixture was kept on the cells for 72 hrs. Then, cells were collected, and total RNA was isolated from the transfected cells using the ReliaPrep™ RNA Miniprep kit. After removal of the culture medium, cells were washed once with PBS. After complete aspiration of the PBS, 100 μL lysis buffer was added to the wells to lyse the cells and collect the intracellular material. After addition of 35 μL isopropanol the mixtures were loaded in a column and subjected to several wash steps and DNase I treatment. After elution in a total volume of 15 μL DNase/RNase-free water, the RNA yield was determined using spectrophotometric analysis (NanoDrop) and stored at −80° C.

Maxima reverse transcriptase (RT, Thermo Fisher) was used to generate complementary DNA (cDNA). Typically, 500 ng total RNA was used in reaction mixture containing 4 μL 5×RT buffer, 2 μL dNTP mix (10 mM each), 0.5 L Oligo (dT), 0.5 μL random hexamer and 0.5 μL Maxima reverse transcriptase (all Thermo Fisher) supplemented with DNase and RNase free water to a total volume of 20 μL. Samples were loaded in a T100 thermocycler (Bio-Rad) and initially incubated at 10 min at 25° C., followed by a cDNA reaction temperature of 30 min at 50° C. and a termination step of 5 min at 85° C. Samples were cooled down to 4° C. prior storing at −20° C.

To determine the editing efficiency, cDNA samples were used in a multiplex digital PCR (dPCR) assay. HepG2 cDNA samples were diluted 5 times before dPCR measurements. The dPCR is designed to distinguish between cDNA species containing the original adenosine or the edited inosine, which is converted into a guanidine during cDNA synthesis. The dPCR also quantifies the amount of B4GALT1 specific cDNA molecules in the mixture using a primer/probe set targeting exons 1 and 2. The primer and probe sequences are listed in Table 1.

TABLE 1
Primer and probe names and sequences (+ refers to an LNA nucleotide
at the 3′ side)

| Name | Sequence 5′ to 3′ |
|---|---|
| hB4GALT1_e05_fw | TCCGCCACTCAAGAGACAAG (SEQ ID NO: 39) |
| hB4GALT1_e06_rv1 | GCACCTGGTAGGTGAGTGAG (SEQ ID NO: 40) |
| hB4GALT1_e01_fw | CCCGCTAGCAACTTGACCTC (SEQ ID NO: 41) |
| hB4GALT1_e02_rv | CCAGGTCCACAGGCATGTTA (SEQ ID NO: 42) |
| hB4GALT1_e05_edit_FAM | /56-FAM/GAACCC+A+G+TCCTCAG+AG/3IABkFQ/ (SEQ ID |
| | NO: 43) |
| hB4GALT1_e05_ori_HEX | /5HEX/A+TGA+ACCC+A+A+TCCTCAG+AG/3IABkFQ/ (SEQ |
| | ID NO: 44) |
| hB4GALT1_e01-02_TEX | /5TEX615/AGGAGTCCCCGCTGCTTGTG/3IAbRQSp/ (SEQ ID |
| | NO:45) |

Digital PCR was performed using the QIAcuity 4, 5-plex, a QIAcuity PCR kit and 96-well 8.5K Nanoplates (Qiagen). In total 1.2 μL of the diluted cDNA mix was used in a dPCR mixture containing 3 μL 4× QIAcuity Mastermix, 0.6 μL per primer (10 μM stock concentration) and 0.3 L per probe (10 μM stock concentration) supplemented with DNase and RNase free water to a total volume of 12 μL. The dPCR mixture was prepared in a pre-plate and then transferred into a 96-well 8.5 K Nanoplate and sealed with a Nanoplate seal. The plate was then transferred to the QIAcuity Four machine. First a priming and rolling step was performed to generate and isolate chamber partitions, followed by an amplification step using the following cycling protocol: 95° C. for 2 min for enzyme activation, 95° C. for 15 sec for denaturation, and 60° C. for 30 sec for annealing/extension for 40 cycles. The amplification step was followed by an image acquisition step of all wells. Data was analysed using the QIAcuity Suite Software (Qiagen).

Percentage of A-to-I editing was determined by dividing the number of G-containing partitions by the total (G-plus A-containing partitions) per ng RNA multiplied by 100.

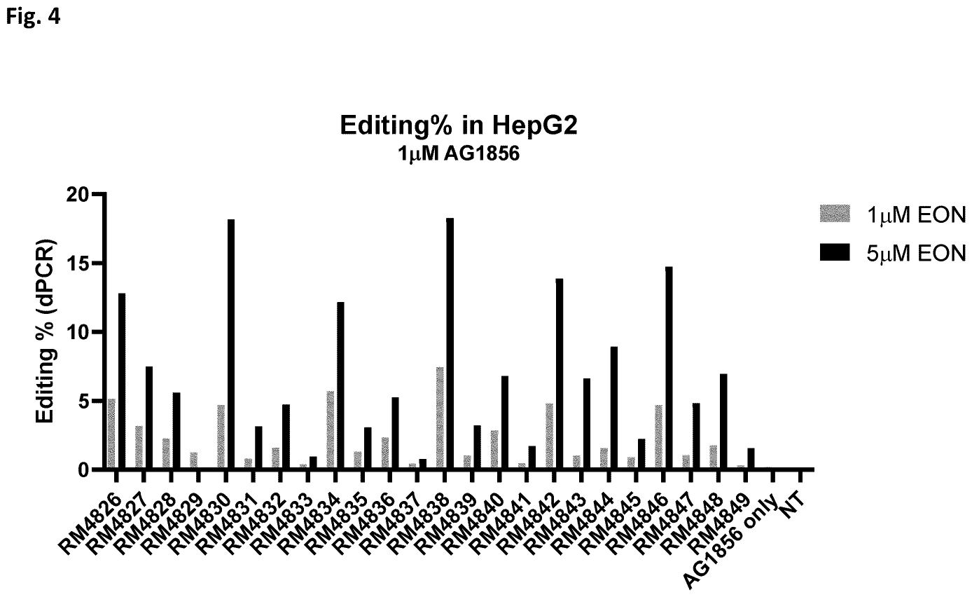

Results of the RNA editing of the endogenous B4GALT1 transcript are provided in FIG. 4, which shows that the efficiency varied significantly between EONs, but that an increase from 1 μm EON to 5 μM EON provided higher editing levels, and importantly, proper editing levels were obtained when the EONs were administered in the context of AG1856. No editing was observed in the negative controls (AG1856 only, and non-treated samples (NT)).

### Example 4. Editing of Human B4GALT1 Transcripts in Liver Spheroids

Next, it was investigated whether RNA editing could also be achieved on endogenous B4GALT1 transcripts in liver spheroids grown from primary human hepatocytes. To generate spheroids, female primary human hepatocytes (PHH) cells (BiolVT) were used. As per the supplier's protocol, 1,500 cells/well of PHH cell suspension (15,000 cells/mL) were plated in a Nuclon Sphera low attachment U-bottom 96 well plates using INVITROGRO Spheroid Plating Medium in combination with Spheroid Medium Supplement A, TORPEDO Antibiotic Mix, and INVITROGRO Spheroid Spin Medium (all from BiolVT). The plates were then incubated for 5 days at 37° C. in a 5% CO₂ atmosphere.

After the incubation, the spheroids were transferred and pooled into Flat-bottom 96 well plates. The pooling resulted in 8 spheroids per well, with a total medium volume of 100 μL/well. To maintain the spheroid cultures, 100 μL of maintenance medium was added to each well and incubated for another 48 hrs at 37° C. in a 5% CO₂ atmosphere. The maintenance medium consisted out of INVITROGRO Spheroid Maintenance Medium (BiolVT), combined with Spheroid Medium Supplement A, which was also used during the treatment of the spheroids. Then, 100 μL of medium was removed from each well (containing the 7-day-old spheroids) and 100 μL of the treatment condition was added. Each well was treated with 1 or 5 μM EON and 1 μM AG1856 saponin for 72 hrs. For this initial spheroid experiment, four of the best performing EONs in cells (see above) were selected: RM4834, RM4838, RM4842, and RM4846. Negative controls were saponin only and NT samples.

After EON/saponin exposure, spheroids were collected, washed once with PBS and then 300 μL lysis buffer was added. RNA isolation, RNA yield determination, cDNA generation, and editing efficiency using dPCR were performed as described above.

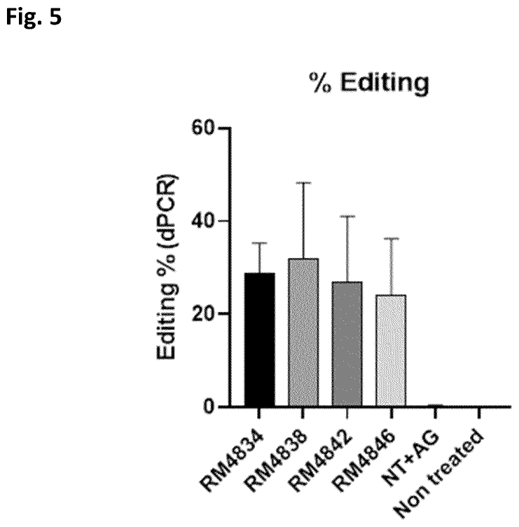

Results are shown in FIG. 5. In line with the results observed in the HepG2 cells, all four EONs could mediate relatively high levels of RNA editing of the endogenous B4GALT1 transcript in the PHH grown spheroids when AG1856 was co-administered, with levels up to more than 30%. All four EONs performed in a similar range.

### Example 5. Editing of Human B4GALT1 Transcripts in Primary Human Hepatocytes

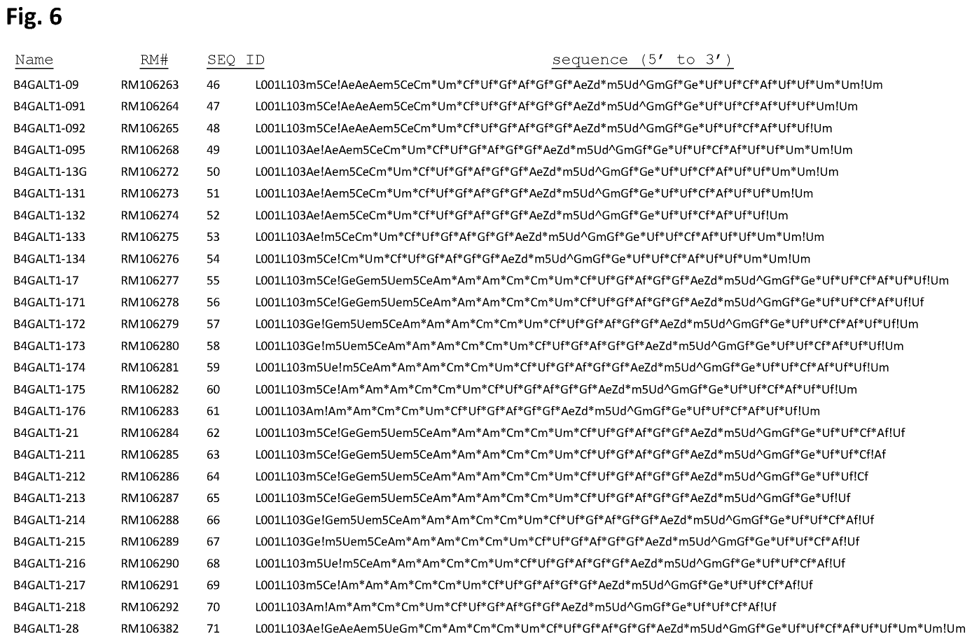

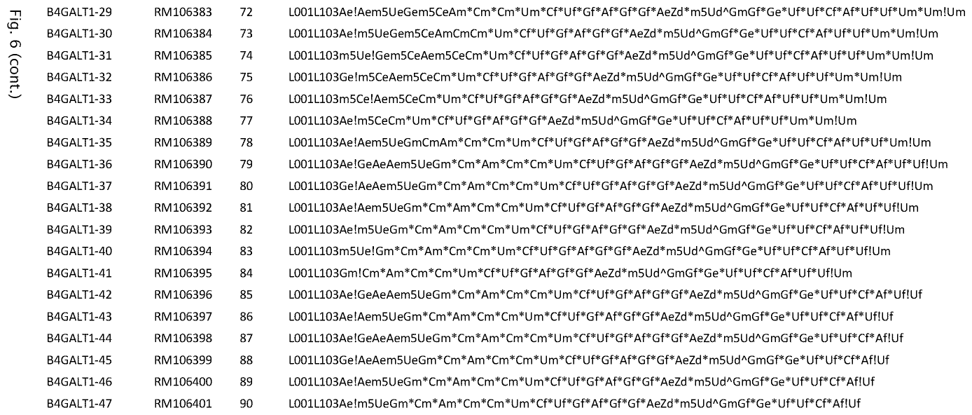

Next, a new set of EONs was designed, based on the best performing editors from the first editing screens (EON01 (RM4826), EON05 (RM4830), RM4834, RM4838, RM4842, and RM4846). This new set of EONs with their respective chemical modifications is given in FIG. 6. Some of these EONs differ in the 5′ terminal part, since some EONs are complementary to exon 6 (hence, after splicing of intron 5 from the pre-mRNA), whereas some are complementary on the 5′ terminal part with intron 5 (hence, before splicing), see for instance the difference in the 5′ terminal parts of B4GALT1-32 (RM106386) and B4GALT1-218 (RM106292). Each one of the EONs in FIG. 6 comprises a tri-antennary GalNAc modification (L001=OP-042; Hongene Biotech) on the 5′ terminus, linked to the most terminal 5′ nucleotide through a TEG linker (L103) to stimulate entry into liver cells. The EONs and their attachments were manufactured according to standard protocols known to the person skilled in the art. The TEG linker was as formula (VI):

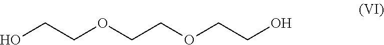

Using the new set of EONs, together with EON01 (RM4826) and EON05 (RM4830), editing of the endogenous human B4GALT1 transcript was investigated in primary human hepatocytes (PHH). A non-treated (NT) sample was taken along as a negative control. For this, a total of 0.5×10⁵ PHH cells (BiolVT) were plated using INVITGRO CP medium supplemented with TORPEDO Antibiotic Mix. Cells were kept at 37° C. in a 5% CO₂ atmosphere. Four hours after plating, the medium was refreshed with cultured in INVITROGRO HI medium supplemented with TORPEDO Antibiotic mix. The next day, cells were treated with 5 μM EON+1 μM AG1856 per well. This mixture was kept on the cells for 72 hrs. Then, the cells were washed once with PBS and 100 μL lysis buffer was added. Total RNA was isolated using the Direct-zol™ RNA Microprep kit (Zymo Research). After the addition of 100 L ethanol (95-100%), the mixtures were loaded onto a column and subjected to several wash steps and DNase I treatment. After elution in a total volume of 15 μL DNase/RNase-free water, the RNA yield was determined using spectrophotometric analysis (NanoDrop) and stored at −80° C. Subsequently, RT reactions and dPCRs were performed as outlined above, with the indicated primers and probes.

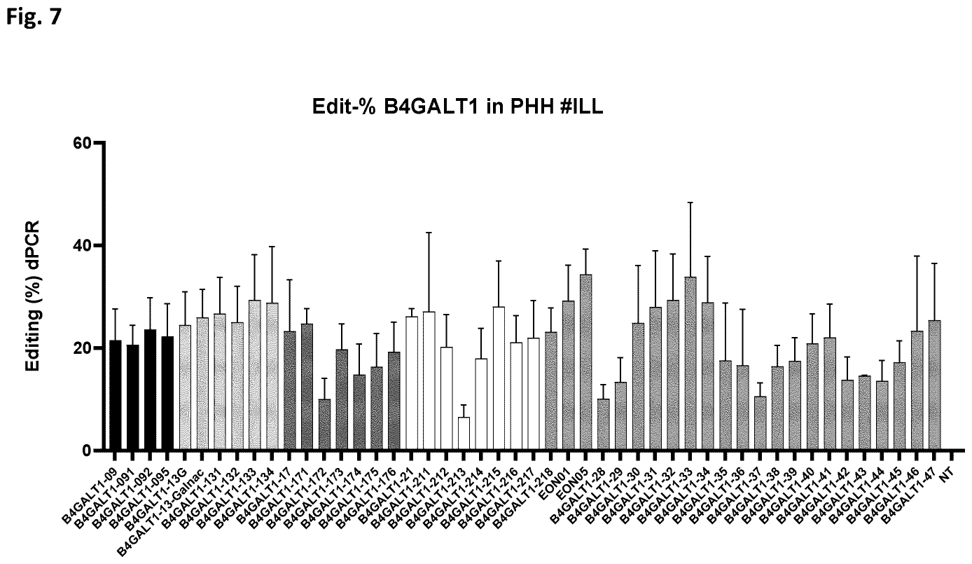

Results are shown in FIG. 7 showing editing levels between 20 and 40% in general for most EONs. To determine the influence of the saponin, it was investigated whether RNA editing could also be achieved in PHHs without adding the saponin, hence by using a simple co-incubation of the oligonucleotides in the medium of the cells (=gymnotic uptake, or ‘gymnosis’). The entire experimental setup was identical to the above setup with the exception that no saponin was added together with the EONs. All downstream RNA purification and dPCR steps were as mentioned above.

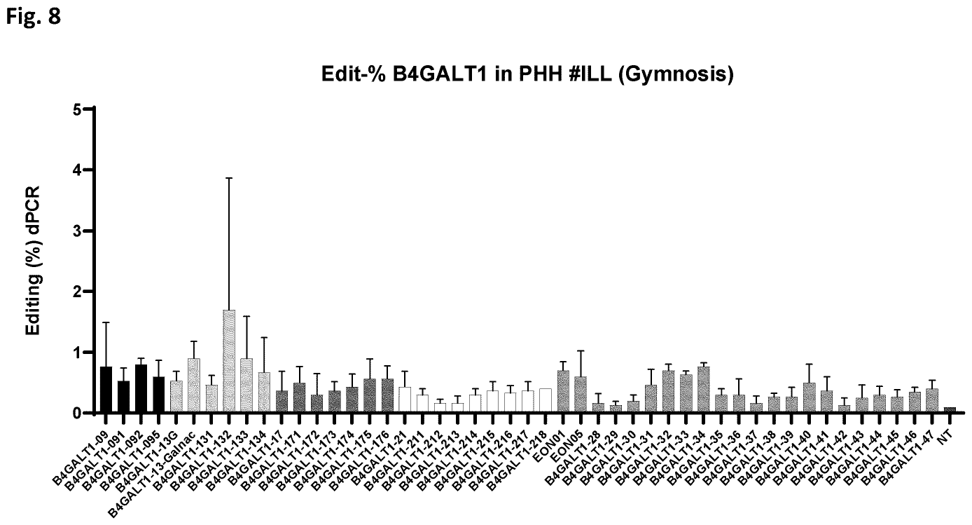

The results of this experiment are shown in FIG. 8, which shows that the percentage RNA editing after gymnotic uptake of the indicated EONs (hence without adding AG1856), was dramatically lower than as shown in FIG. 7, clearly indicating the positive effect of the AG1856 co-administration to achieve RNA editing on a different sequence, in this case the human B4GALT1 transcript.

### Example 6. Editing of a Target Adenosine in an Endogenous ANGPTL3 Target RNA Molecule in Human Huh-7 Cells and Liver Spheroids

Elevated plasma levels of Low-Density Lipoprotein Cholesterol (LDL-C, also known as “bad” cholesterol) are well-known risk factors for atherosclerotic cardiovascular disease (ASCVD). The strong causal association between plasma LDL-C and ASCVD forms the basis for the use of aggressive LDL-lowering therapies in individuals at high risk of ASCVD. Considerable attention has been focused on the enzyme lipoprotein lipase (LPL), which catalyses the hydrolysis of plasma triglycerides and is rate limiting for triglyceride uptake into muscle, heart, and adipose tissue. Due to its importance in plasma lipid metabolism, the activity of LPL in different tissues is carefully regulated to be able to cater lipid uptake to local lipid demand. Angiopoietin-like 3 (ANGPTL3) protein is one of the proteins involved in the regulation of lipoprotein lipase (LPL) activity. The use of nucleotide editing technology in targeting the gene transcript encoding of the Angiopoietin-like 3 (ANGPTL3) protein could bring about amino acid changes that yield an ANGPTL3 protein with a reduced ability to inhibit lipolysis.

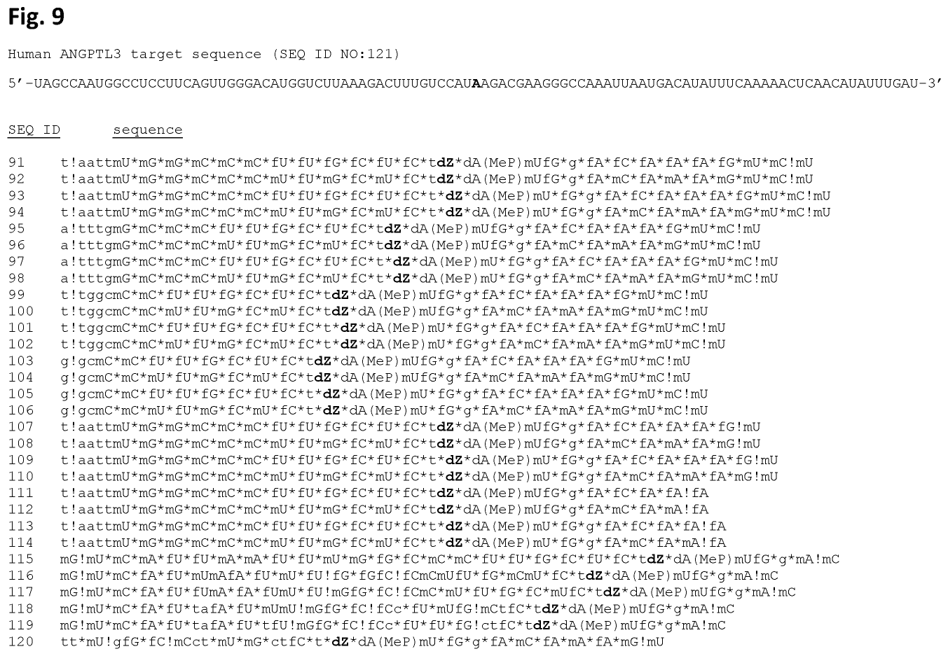

A set of 30 ANGPTL3-targeting EONs (see FIG. 9, wherein SEQ ID NO:91 to 120 relate to RM5035 to RM5064, respectively) were tested to assess the levels of editing of human ANGPTL3 target (pre-) mRNA in cells after gymnotic incubation with the EONs (gymnotic=without any transfection means), and in the context of the saponin AG1856. For this, human Huh-7 hepatocyte-derived cellular carcinoma cells (CLS Cell Lines Service GmbH) were cultured in RPMI 1640 supplemented with 10% FBS/2 mM L-glutamine and kept at 37° C. in a 5% CO₂ atmosphere. Primary human hepatocytes (BiolVT) were cultured in complementary INVITROGRO Plating and Maintenance Medium (BiolVT) and kept at 37° C. in a 10% CO₂ atmosphere. Human primary hepatocytes derived liver-spheroids were generated using the manufacturer's (BiolVT) protocol. A total of 0.5×10⁵ Huh-7 cells were seeded in wells of a 24-well plate one day prior exposure to the EONs. After ON incubation, plating medium was aspirated and mixtures of EON in fresh culture medium were added to the cells. In experiments where the saponin AG1856 was added, the mixture containing 1 μM EON was supplemented with 1 μM AG1856. Cells were incubated with the inoculates for 72-hrs before total RNA isolation. For the gymnotic treatment of the human primary hepatocyte derived spheroids, 1.5×10³ cells were seeded in a 96-well plate in a total volume of 100 μL plating medium. Plates were subjected to 2 min of 250 g centrifugation to accumulate the cells to the bottom of the well. Spheroids were formed over 5 days of incubation. Before exposure, medium was aspirated and mixtures containing 5 μM EON and 1 μM AG1856 in fresh maintenance medium were added. After 72 hrs incubation, the medium was aspirated, and total RNA was isolated. 72 hours post exposure to the EONs, cells were collected, and total RNA was isolated from the cells using the Direct-zol RNA Microprep kit (Zymo Research). After removal of the culture medium, the cells were washed once with PBS. After complete aspiration of the PBS, 100 μL TRIreagent (Zymo Research) was added to lyse the cells and collect the intracellular material. For the spheroids, 300 μL of TRIreagent was used. After addition of 100 μL ethanol (300 μL for the spheroid samples), the mixtures were loaded in a column and subjected to several wash steps and DNasel treatment. After elution in a total volume of 15 μL DNase/RNase-free water, the RNA yield was determined using spectrophotometric analysis (NanoDrop) and stored at −80° C. Maxima Reverse Transcriptase (RT, ThermoFisher) was used to generate cDNA. Typically, 100 nanogram total RNA was used in a reaction mixture containing 4 L 5× RT buffer, 1 μL dNTP mix (10 mM each), 0.5 μL Oligo (dT), 0.5 μL random hexamer (all ThermoFisher) supplemented with DNase- and RNase-free water to a total volume of 20 μL. Samples were loaded in a T100 thermocycler (Bio-Rad) and initially incubated at 10 min at 25° C., followed by a cDNA reaction temperature of 50° C. (30 min) and a termination step of 5 min at 85° C. Samples were cooled down to 4° C. prior storing at −20° C.

To determine the editing efficiency, cDNA samples were used in multiplex dPCR (Qiagen) assays. The first assay was designed to distinguish between cDNA species containing the original adenosine or the edited inosine (which is converted into a guanidine during cDNA synthesis and subsequent PCR). The second multiplex ddPCR quantifies the amount of ANGPTL3 transcripts measuring exon 6-7 specific fragments. A separate HPRT1-specific dPCR was used to correct for variation in sample isolation or possible effects during exposure, using a HPRT1-specific primer/probe set. The primer and probe sequences are listed in Table 2, the cycling conditions in Table 3.

TABLE 2
primer and probe names and sequences (+ refers to an LNA nucleotide
at the 3′ side)

| Name | Sequence 5'-3' |
|---|---|
| hANGPTL3_e01_Fw1 | AATGGCCTCCTTCAGTTGGG (SEQ ID NO: 62) |
| hANGPTL3_e01_Rv1 | TGATTTCACTGGTTTGCAGCG (SEQ ID NO: 63) |
| hANGPTL3_e01_FAM | CTTTGTCCA+T+G+AGACGAAG (SEQ ID NO: 64) |
| hANGPTL3_e01_HEX | CTTTGTCCA+T+A+AGACGAAGG (SEQ ID NO: 65) |
| hANGPTL3_e06_Fw1 | CGATTACTGGCAATGTCCCC (SEQ ID NO: 66) |
| hANGPTL3_e07_Rv1 | TCTCCACACTCATCATGCCAC (SEQ ID NO: 67) |
| hANGPTL3_e06_TEX | ACTTCAACTGTCCAGAGGGT (SEQ ID NO: 68) |
| HPRT1 fw | CCTGGCGTCGTGATTAGTGA (SEQ ID NO: 69) |
| HPRT1 rv | CGAGCAAGACGTTCAGTCCT (SEQ ID NO: 70) |
| HPRT1 TEX red | TGC+TGAGGATTTGGAAAGGGTGT (SEQ ID NO: 71) |

TABLE 3
ddPCR cycling conditions

| | | | |
|---|---|---|---|
| Step | Temperature | Time | Cycles |

| | | | | | |
|---|---|---|---|---|---|
| Enzyme activation | 95 | °C | 2 | minutes | 1 |
| Denaturation | 95 | °C | 15 | seconds | 40 |
| Annealing/extension | 60 | °C | 30 | seconds | |

| | | | | |
|---|---|---|---|---|
| Hold | 4 | °C | Indef. | ∞ |

In total, 1 μL of the cDNA mix was used in a dPCR mixture containing 3 μL 4× dPCR QIAcuity Mastermix for probes (Qiagen), 0.6 μL primers and 0.3 μL probes (10 μM stock concentration each), supplemented with 4.5 μL DNase- and RNase-free water in a total volume of 12 μL. The resulting mixture was mixed thoroughly and transferred to a well of a QIAcuity 96-wells 8.5K Nanoplate (Qiagen) and loaded in a QIAcuity dPCR machine. Data was analyzed using the QIAcuity Software Suit (Qiagen). Percentage of A-to-I editing was determined by dividing the number of G-containing molecules by the total (G-plus A-containing species) multiplied by 100.

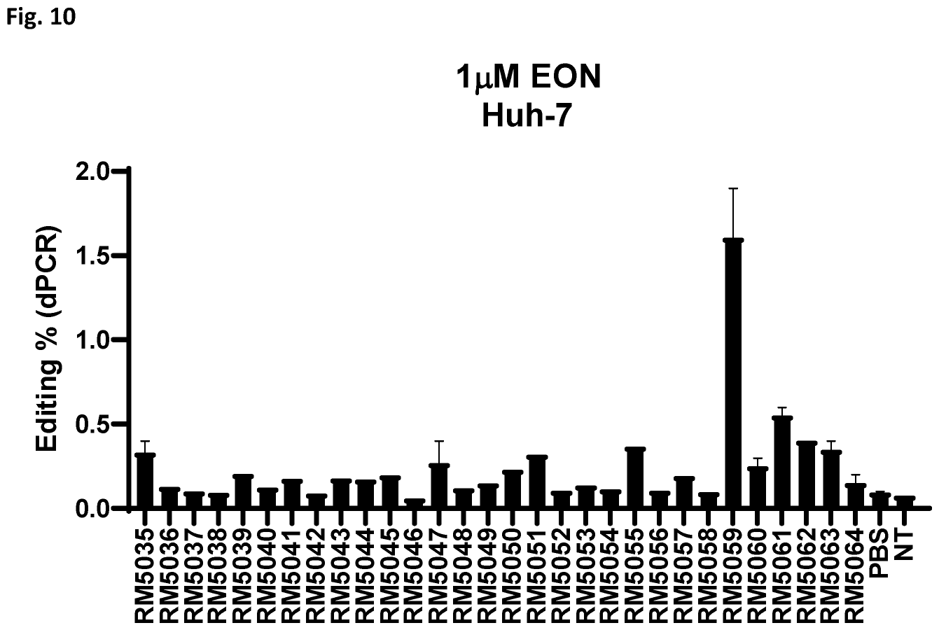

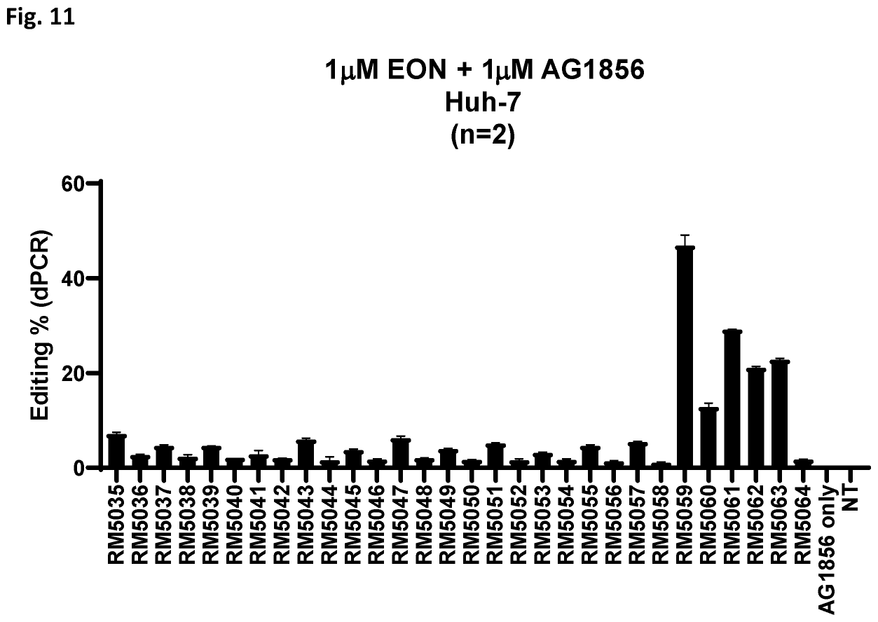

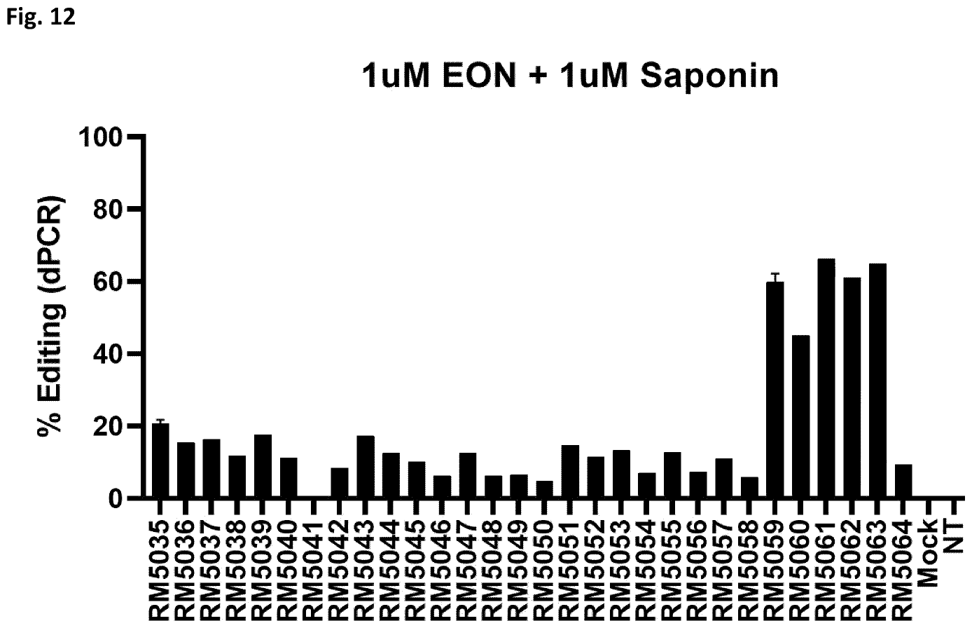

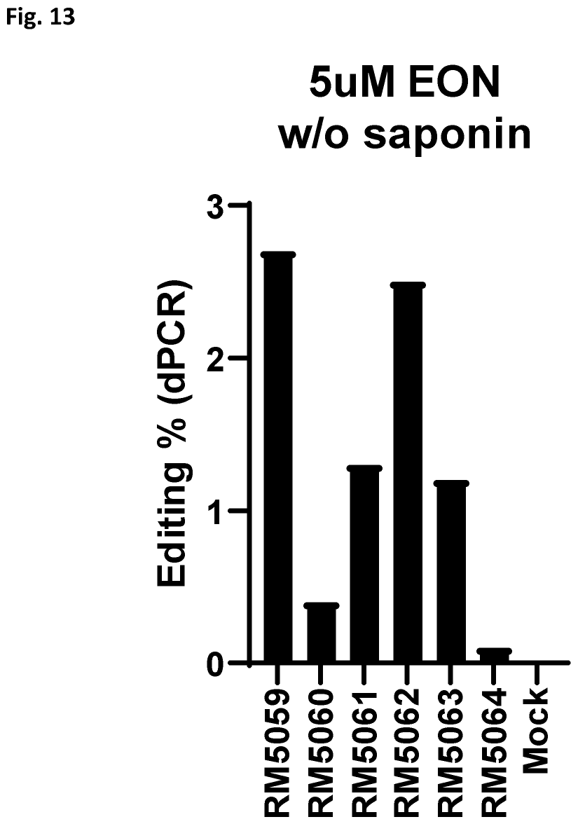

FIG. 10 shows the percentage A to I editing as determined in human Huh-7 cells after incubation with the 30 indicated EONs using a gymnotic approach in which no saponin was applied, as outlined above. Most EONs showed detectable but low editing levels. FIG. 11 shows the results of an identical experiment, but now wherein the Huh-7 cells were co-incubated with the triterpene glycoside AG1856. Clearly, no editing could be observed in the non-treated (NT) sample and the AG1856 alone control, but editing levels were dramatically increased in comparison to the experiment in which no saponin was used. Some editing levels were as high as 60%, clearly indicating the beneficial properties of the co-administration of the saponin. FIG. 12 shows the results of the experiment in which the same 30 EONs were tested for A to I editing of ANGPTL3 transcripts in liver spheroids, generated from primary human hepatocytes. All incubations of EONs were accompanied by incubation with 5 μM AG1856, and all EONs (except for RM5041, which likely resembles an experimental error because editing was found for this EON in the Huh-7 cells) showed very significant levels of editing, with editing percentages of more than 60% of the ANGPTL3 transcripts. To check for the influence of the AG1856 saponin, the same experiment was performed in liver spheroids generated from primary human hepatocytes using a 5 μM incubation of six selected EONs: RM5059 (SEQ ID NO:115), RM5060 (SEQ ID NO:116), RM5061 (SEQ ID NO:117), RM5062 (SEQ ID NO:118), RM5063 (SEQ ID NO:119), and RM5064 (SEQ ID NO: 120), but without the addition of the saponin. The results are shown in FIG. 13 and indicate that RNA editing of the target adenosine in the endogenous ANGPTL3 transcript could be achieved but were far less in the absence of the saponin, again confirming the beneficial effect of co-administration of the saponin.

### Example 7. Editing of a Target Adenosine in an Endogenous Actin B Target RNA Molecule In Vivo

Similar to example 2, a study was performed to investigate the beneficial properties of using AG1856 in an in vivo RNA editing experiment, now targeting the endogenous mouse Actin B (mActB) target transcript. For this, female C57BL/6J mice (n=4-6, 9-16 wk old) were given 5 daily doses of EON RM3891 that targets wild-type mouse Actb (10 mg/kg at each day 1-5) or PBS with or without a single subcutaneous (SC) dose of AG1856 (3.6 mg/kg at day 5, 1 h post RM3891 treatment), all by SC administration. Animals were sacrificed at 3 days post last dose (=at day 8) and tissues were isolated. RM3891 has the following sequence (5′ to 3′; SEQ ID NO: 132; with the chemical modifications as given in FIG. 3):

Um!Cm*Cm*Um*Gm*Um*Am*Af*Cm*Cf*Am*Cf*m5UeZd*
Ad∧UmUf*m5Ue*Cf*Am*Uf*Gm*Gf*Am*Um*Am!Cm

Snap frozen tissue samples were thawed and disrupted in TRIzol reagent (Roche) using the MagNA lyzer (Roche). Samples were exposed to two MagNA lyzer runs of 30 sec each (6500 rpm), allowing for a 90 sec cool-off period in between runs. Samples were then incubated for 2 min at RT to allow complete dissociation of nucleoproteins. Subsequently, chloroform was added to the suspension preparing for phase separation. After centrifuging for 15 min at 12.000 g (4° C.), the aqueous phase that contained RNA was used for further processing. The ReliaPrep RNA Cell Miniprep System was used to isolate RNA following manufacturer's protocol. cDNA synthesis was performed using the Maxima Reverse Transcriptase kit (Thermo Scientific) following manufacturer's protocol. In short, to avoid cDNA synthesis interference due to secondary structures, 500 ng RNA was first incubated with dNTP mix (10 mM each), random hexamers and oligoDt at 65° C. for 5 min, then slowly cooled to 10° C. in 10° C. per 15 sec declines. Subsequently, reverse transcriptase buffer and enzyme was added, and samples were incubated 10 min at 25° C. for 10 min, 50° C. for 30 min, and 80° C. for 5 min. For dPCR analysis, cDNA samples were incubated with a mix of primers and probes specific for wild type mActB 3′-UTR (HEX), mutant mActB 3′-UTR (FAM), total mActB exon 2-3 (Cy5) and household gene mRps19 (Table 4). 12 μl of each sample was loaded onto a QIAcuity nano plate 8.5K 24 wells and run on the QIAcuity (Qiagen) which includes sample partition, PCR (Table 2), and imaging.

TABLE 4
primers and probes (+ refers to an LNA nucleotide at the 3′ side)

| Target | Primer sequence (5′-3′) | Probe sequence (5′-3′) |
|---|---|---|
| mActB 3′-UTR | (Fw): AATAGTCATTCCAAGTAT CCATG | Wild type: /5HEX/AA+C +CAC +T+T+A T+TT |
| | (SEQ ID NO: 122) | CA+T G/3IABkFQ/ (SEQ ID NO: 128) |
| | (Rv): GCTTTTGGGAGGGTGA | Mutant: /56-FAM/AA+C CAC +T+C+A T+TT |
| | (SEQ ID NO: 123) | +CAT G/3IABkFQ/ (SEQ ID NO: 129) |
| mActB exon | (Fw): GGCTGTATTCCCCTCCA | /5Cy5/TGG TGG GAA TGG GTC AGA AGG |
| 2-3 | (SEQ ID NO: 124) | A/3IAbRQSp/ (SEQ ID NO:130) |
| | (Rv): GTTCAATGGGGTACTTCAGG | |
| | (SEQ ID NO: 125) | |
| mRps19 | (Fw): CGAGCTGCTTCCACA | /5TexRd-XN/GC ACC TGT ACC TCC GAG GTG |
| | (SEQ ID NO: 126) | /3BHQ_2/ (SEQ ID NO: 131) |
| | (Rv): GCCACACTCTTAGAGCC | |
| | (SEQ ID NO: 127) | |

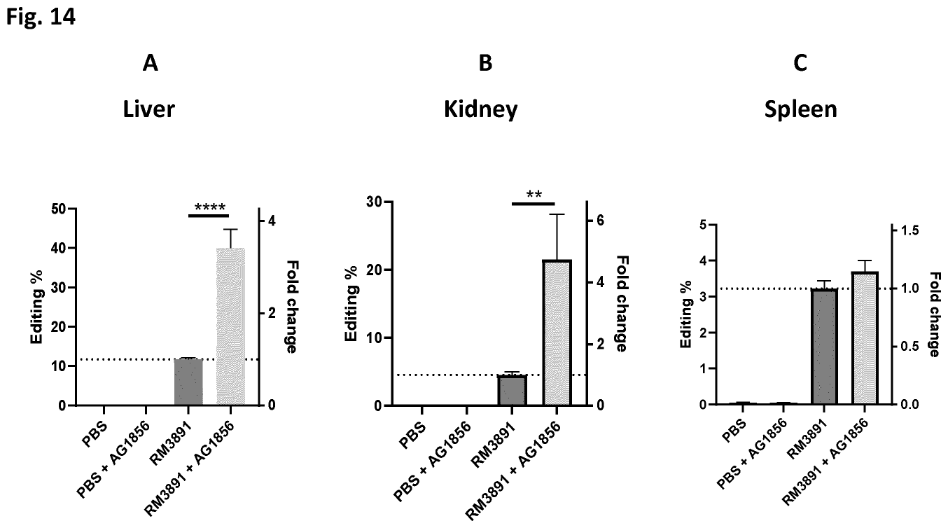

The results of these in vivo experiments are given in FIG. 14A: liver, FIG. 14B: kidney, and FIG. 14C: spleen. No editing could be observed in the control mice (PBS alone or PBS+AG1856), whereas significant editing percentages of the endogenous mActB transcript could be detected in all three tissues when using RM3891 alone, which was then significantly increased in liver and kidney when AG1856 was co-administered, again confirming the results obtained in vitro, as outlined above, and in vivo, as outlined above and shown in FIG. 2.

### Example 8. Editing of a Target Adenosine in a mApp Target RNA Molecule in Primary Mouse Hepatocytes Using AG1856-EON Conjugates

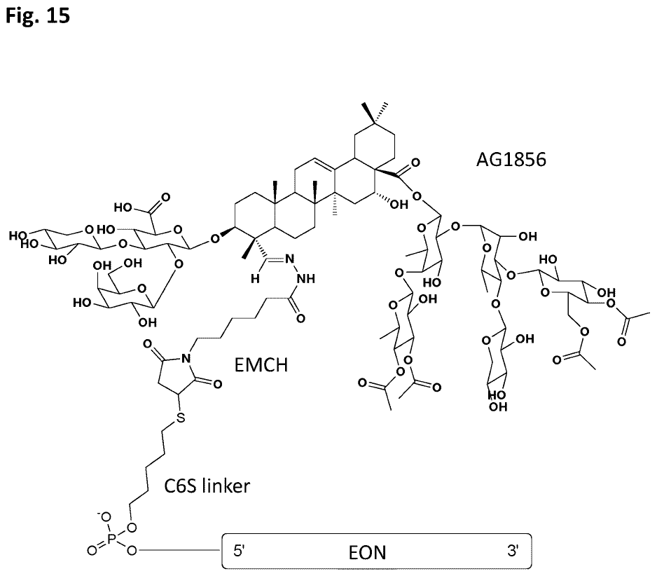

Next, it was investigated whether conjugating the saponin molecule to an RNA editing oligonucleotide could further improve the editing efficiency and/or levels. The structure that was initially prepared is shown in FIG. 15, that indicates that the saponin (AG1856) is conjugated to the 5′ terminus of the EON, using an N-E-maleimidocaproic acid hydrazide (EMCH) group initially attached to the saponin and a C6S linker, which was initially attached to the EON.

The AG1856-EMCH part was prepared as follows. Stock solutions of AG1856 (Clochard et al. 2020) in EtOH (672.8 μM) and EMCH hydrazide, trifluoroacetic acid salt) in CHCl₃ (17.7 mM) were prepared. EMCH (3 eq) was added to the AG1856 solution (1 eq) and incubated at RT for 24 hrs. The reaction was monitored by thin-layer chromatography (CHCl₃/MeOH/H₂O/CH₃COOH, 50:40:10:5, staining: vanillin). The final product was purified by HPLC (C₁₈, 70% H₂O (+0.01% TFA)/MeCN (+0.01% TFA) for 10 min, to 42% in 5 min, then to 35% in 20 min). Solvents were removed by speedvac followed by lyophilization to give typical yields of 75%.

The EON-C6S part was generated using standard methods known to the person skilled in the art, in which the thiol modifier C6 S-S of formula (VI):

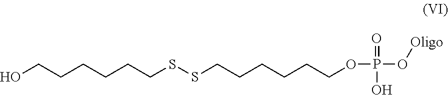

was attached to the 5′ terminus of the EON. The EON that was selected for editing the target adenosine in the mouse App (mApp) transcript (see Example 1) was RM5522 that has the following sequence (5′ to 3′; SEQ ID NO: 133; with the chemical modifications as given in FIG. 3, and it which L101 represents the C6S-S linker, attached to the 5′ terminus 2′-OMe modified adenosine (Am) by a phosphodiester bond):

L101^{θ}Am!Um*Cm*Am*Cm*Gf*Gm*Uf*Um*Gf*m5Ce*Zd*
Ad∧Um*Gf*Ae*Cf*Am*Af*Cm*Gf*Cm*Cm*Gm!Cm

The conjugation of AG1856-EMCH and EON-C6-6 comprises two reaction steps: i) Reduction of the disulfide, and ii) Michael Addition. The reduction was performed as follows. Degassed PBS buffer (10 mL portions) was prepared by sparging with nitrogen for 25-30 min.

The EON-C6S was prepared to 0.164 μmol/160 μL degassed PBS, and a Tris (2-carboxyethyl) phosphine hydrochloride (TCEP; CAS: 51805-45-9)-stock solution was prepared to 1.64 μmol/40 μL degassed PBS. All stock solutions were flushed with nitrogen until usage. 160 μL RM5522-stock solution (1.0 eq., 0.164 μmol, 1.5 mg) and 40 μL TCEP-stock solution (10.0 eq., 1.64 μmol, 0.47 mg) were mixed in a 2 mL-reaction tube equipped with a stirring bar, flushed with nitrogen, and stirred at ambient temperature for approximately 90 min. To maintain the reduced state of the EON, in all workups every tube or filter was thoroughly flushed with nitrogen after opening before it was closed again. Amicon Ultra-4 centrifugal filters (3K) were pre-rinsed with 2 mL PBS for approximately 30 min while centrifuging. After the reaction, the reaction mixture was diluted with 1600 μL degassed PBS and the stirring bar was removed. To remove the excess of TCEP and remainers of free thiols from the RM5522-SS-protection group, the diluted reaction mixture (1800 μL) was transferred to the pre-rinsed Amicon Ultra centrifugal filters and spun for approximately 40 min. The remaining solution was diluted one additional time with 1800 μL degassed PBS and again concentrated for approximately 40 min with centrifugation. The residual solution was diluted to 160 μL with degassed PBS, collected with a pipette and transferred to the Michael Addition-reaction vessel.

The Michael Addition was performed as follows. First, the AG1856-EMCH maleimide-stock solution was prepared to 0.328 μmol/40 μL in anhydrous N,N-dimethylformamide (DMF; CAS: 68-12-2) and flushed with nitrogen until use. In a 2 mL reaction tube with seal and equipped with a stirring bar, the EON with the free thiol group (the worked-up reaction mixture of the reduction step) was mixed with 40 μL (2.0 eq., 0.328 μmol, 0.677 mg) AG1856-maleimide stock solution, flushed with nitrogen, covered with parafilm, and stirred at ambient temperature overnight (appr. 15 hrs). PBS with a pH 6.9 was prepared by adding HCl to a PBS stock solution. Amicon Ultra-4 centrifugal filters (3K) were pre-rinsed with 1.8 mL PBS pH 6.9 for 40 min. The reaction mixture was diluted with 1600 μL PBS pH 6.9 to lessen the percentage of DMF which is not tolerated by the filter membrane. The diluted reaction mixture was then transferred to the Amicon filters and concentrated (for removal of DMF and excess maleimide) for >40 min. The remaining solution (around 100 μL) was diluted an additional three times with 1800 μL PBS and concentrated for 45 min. The residual solutions were diluted to 250 μL with PBS pH 6.9 and collected with a pipette. Mass analysis and native PAGE were performed on the crude product using standard procedures known to the person skilled in the art, which confirmed conjugation of the EON to the AG1856 saponin (data not shown).

To investigate whether an EON conjugated to a saponin (in the case exemplified by AG1856) and prepared as outlined above, would still be able to generate RNA editing (and therefore: recruite endogenous ADAR) in cells, the conjugate was compared to an administration of RM5522 alone (and RM3835, see example 2), and was further compared to an administration of RM5522 with separately AG1856 and in comparison to RM5522 alone.

Mouse hepatocytes were isolated using the GentleMACS Dissociator and liver perfusion kit (130-128-030) from Miltenyi Biotec, following the manufacturer's protocol. In short, livers were perfused on the GentleMACS Dissociator by running the LIPK_HR-1 program where the livers were washed with a pre-digestion buffer and lastly perfused with an enzyme digestion solution. Thereafter the livers were dissociated on the GentleMACS Dissociator by transferring them to a C Tube along with the digestion solution from the previous step and running the LIPK_HR-1 program. To achieve a single-cell suspension the dissociated liver solution was poured through a 70 μM strainer, carefully centrifuged and resuspended in DMEM solution (low glucose, no glutamine, no phenol red with 5% FBS and 1% pen/strep medium). For further hepatocyte enrichment a debris removal step was performed with a density gradient centrifugation using a debris removal solution. The supernatant was carefully removed, and the cell pellet was resuspended in DMEM solution. For the A to I editing cell assay, primary mouse hepatocytes were seeded in a Collagen I coated 24-well plate at a density of 7.5×10⁴ cells per well in 500 μL of DMEM solution and incubated overnight at 37° C. with 5% CO₂. The treatments were:
1 μM RM3835 (EON; see example 2);
1 μM RM5522 (EON);
1 μM RM5522@AG1856 (EON conjugated with AG1856);
0.5 μM RM5522@AG1856;
1 μM RM5522+0.5 μM AG1856 (EON+AG1856 co-treatment); and
0.5 μM RM5522+0.2665 μM AG1856.

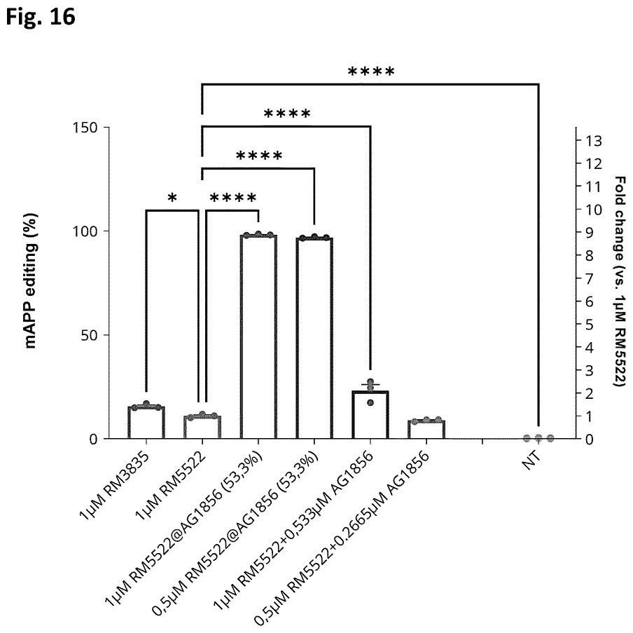

These treatment solutions were prepared in Williams E medium with 2 nM glutamine and 1% pen/strep. After 24 h settlement of the cells, the medium was removed and 500 μL of the above treatment solutions was added to the respective wells. Untreated cells were used as a control. The cells were then incubated for 72 hrs at 37° C./5% CO₂ before RNA isolation. The RNA isolation procedures, the cDNA synthesis, dPCR and primers and conditions were as described in Example 2. The results of the RNA editing assessment following the described treatments is given in FIG. 16. The editing percentages detected after treatment with RM3835 and RM5522 alone (=gymnotic treatment) were approximately 10-15%. However, the editing percentage that was obtained using either 1.0 or 0.5 μM of the RM5522@AG1856 conjugate compound reached a surprising significant high level of almost 100%, indicating that not only all cells were likely hit by the conjugate, but that also all mApp transcripts in the target cells were targeted and all target adenosines were deaminated. Notably, when the RM5522 EON was administered in a concentration of 1 μM together (but not conjugated) with AG1856, editing levels were significantly higher than when AG1856 was not co-administered, around 25%, which was importantly lower than what was observed with the conjugate. These results show that co-administration of a saponin, exemplified by AG1856 herein, with an EON boosts the RNA editing effect brought about by the EON, but that when the EON is conjugated to the saponin, that editing is almost complete, reaching levels of 100% in an in vitro setting, holding promise for much higher editing percentages in vivo as compared to what has been observed when the saponin and the EON were administered in a non-conjugated manner. The investigators have herein shown that RNA editing using chemically modified EONs can be increased when a saponin is co-administered, in vitro but also in in vivo settings, providing significant editing levels in organs such as the liver and the kidney, but foremost have shown that editing levels could be dramatically increased when the EON was conjugated to the EON. Importantly, this shows that the AG1856, while attached to the oligonucleotide, does not hamper enzymatic deamination activities of the recruited endogenous ADAR enzyme in the cell.

1. A composition comprising a triterpene glycoside and an RNA editing producing antisense oligonucleotide (EON), wherein the EON can form a double-stranded complex with a region of a target RNA molecule in a cell, wherein the region of the target RNA molecule comprises a target adenosine, wherein the nucleotide in the EON that is opposite the target adenosine is the orphan nucleotide, and wherein the double-stranded complex can bind an endogenous ADAR enzyme to deaminate the target adenosine into an inosine, thereby editing the target RNA molecule.

2. A composition according to claim 1, wherein the triterpene glycoside is conjugated to the EON.

3. A composition according to claim 1 or 2, wherein the triterpene glycoside is AG1856.

4. A composition according to any one of claims 1 to 3, wherein the orphan nucleotide is a cytidine, a cytidine analog, a cytidine derivative, a uridine, a uridine analog, or a uridine derivative.

5. A composition according to any one of claims 1 to 4, wherein at least one nucleotide in the EON comprises one or more non-naturally occurring chemical modifications in the ribose, the linkage, or the base moiety, with the proviso that the orphan nucleotide is not a cytidine comprising a 2′-OMe ribose substitution.

6. A composition according to any one of claims 1 to 5, wherein the orphan nucleotide is a deoxynucleotide.

7. A composition according to any one of claims 1 to 6, wherein the target RNA molecule is pre-mRNA or mRNA.

8. A composition according to any one of claims 1 to 7, wherein the endogenous ADAR enzyme is human ADAR1, ADAR2 or ADAT.

9. A composition according to any one of claims 1 to 8, for use in the treatment of a cardiovascular disease, a disease involving the liver, a disease involving the kidney, a disease involving the pancreas, or a disorder of the central nervous system.

10. A composition according to any one of claims 1 to 9, wherein the target RNA molecule is endogenously present in the cell and wherein the target RNA molecule is transcribed from a human gene selected from the group consisting of: SERPINA1, IDUA, HFE, ABCA4, USH2A, PCSK9, B4GALT1, ALDH2, HTT, DMD, PNPLA3, APOC3, C9orf72, DMPK, RHO, MAPT, OTOF, SMN1, ASL, APP, ANGPTL3, NTCP, PMP22, LRRK2, ASS1, GJB2, MECP2, and RS1.

11. A kit-of-parts comprising (i) a first pharmaceutical composition comprising a triterpene glycoside, preferably AG1856; and (ii) a second pharmaceutical composition comprising an EON, wherein the EON can form a double-stranded complex with a region of a target RNA molecule in a cell, wherein the region of the target RNA molecule comprises a target adenosine, and wherein the double-stranded complex can recruit an endogenous ADAR enzyme to deaminate the target adenosine into an inosine, thereby editing the target RNA molecule.

12. A method for editing a target adenosine present in an endogenous target RNA molecule in a cell in a subject, comprising the steps of:
(i) administering to said subject a triterpene glycoside, preferably AG1856; and
(ii) administering to said subject an EON, wherein the EON after administration, can form a double-stranded complex with a region of the endogenous target RNA molecule comprising the target adenosine in the cell, and wherein the double-stranded complex can recruit an endogenous ADAR enzyme to deaminate the target adenosine into an inosine.

13. A method according to claim 12, wherein the triterpene glycoside is conjugated to the EON

14. A method according to claim 12 or 13, wherein the cell is a liver cell, a kidney cell, or a neuron.

15. A method for the deamination of a target adenosine in a target RNA molecule, preferably a pre-mRNA or mRNA molecule, in a cell, the method comprising the steps of:
(i) providing the cell with a conjugate comprising:
a triterpene glycoside, preferably AG1856; and
an EON that is bound to the triterpene glycoside at the 3′ or 5′ terminus, wherein the EON can form a double-stranded complex with the target RNA molecule, or a region thereof, wherein the region comprises the target adenosine;
(ii) allowing uptake by the cell of the conjugate;
(iii) allowing annealing of the EON to the target RNA molecule;
(iv) allowing an endogenous ADAR enzyme to deaminate the target adenosine in the target RNA molecule to an inosine; and optionally
(v) identifying the presence of the inosine in the target RNA molecule.
